(19) United States
(12) Patent Application Publication
(10) Pub. No.: US 2022/0008567 A1
(43) Pub. Date: Jan. 13, 2022

(54) LIQUIDLY INJECTABLE, SELF-STABILIZING BIOPOLYMERS FOR THE DELIVERY OF RADIONUCLIDE

(71) Applicant: Duke University, Durham, NC (US)

(72) Inventors: Ashutosh Chilkoti, Durham, NC (US); Jeffrey L. Schaal, Durham, NC (US); Wenge Liu, Durham, NC (US)

(21) Appl. No.: 17/294,368

(22) PCT Filed: Nov. 13, 2019
(86) PCT No.: PCT/US2019/061144

§ 371 Date: May 14, 2021

Related U.S. Application Data
(60) Provisional application No. 62/767,736, filed on Nov. 15, 2018.

(51) Int. Cl. A61K 51/08; A61P 35/00

(52) U.S. Cl. CPC A61K 51/088; A61P 35/00

(57) ABSTRACT

Described herein are compositions for liquidly injectable, self-stabilizing biopolymers for the delivery of radionuclide brachytherapy. Also described herein are methods of using the compositions.

### STATEMENT REGARDING FEDERALLY-SPONSORED RESEARCH OR DEVELOPMENT

This invention was made with government support under grant number R01CA138784-03, R01EB000188, and R35CA197616 awarded by the National Institutes of Health. The government has certain rights in the invention.

### TECHNICAL FIELD

The present disclosure relates to novel liquidly injectable, self-stabilizing biopolymers for the delivery of radionuclide brachytherapy.

### BACKGROUND OF THE INVENTION

Pancreatic cancer is one of the deadliest manifestations of cancer in clinical oncology. Despite accounting for only 3.2% of all cancer cases, it is the third leading cause of cancer-related mortality. The extreme resistance of pancreatic cancer to conventional therapies arises from a convergence of factors unique to pancreatic tumors. These tumors develop genetic aberrations that promote an aggressive phenotype as well as resistance to chemoradiation treatment. Furthermore, the microenvironment of pancreatic tumors consists of an extensive, dense desmoplastic stroma that is hypovascular. Together, they present formidable transport barriers to the delivery of drugs and inhibit existing chemo-radiation therapeutics. In addition, the anatomical location of the pancreas, proximal to hollow viscus organs, itself presents a challenge due to limitation of the typical radiation dosage.

National Comprehensive Cancer Network guidelines recommend external beam radiotherapy (EBRT) combined with chemotherapy as the first-line treatment for locally advanced tumors. EBRT is typically administered concurrently with either gemcitabine or Abraxane, a formulation of paclitaxel bound to human serum albumin. EBRT is also employed as a neoadjuvant therapy for patients who receive surgical resection, accounting for ˜40% of total pancreatic cancer diagnoses. For all its clinical benefits, EBRT possesses significant limitations in the clinical management of pancreatic cancer. It is even less effective for treating cancers where distal metastases have developed. EBRT also exposes adjacent healthy tissue to ionizing radiation. This exposure can induce serious side effects, particularly in hollow viscus organs, such as the duodenum and stomach. The frequency and intensity of the radiation dose must therefore be limited as a safety precaution, which often renders treatment ineffective for tumors that have high intrinsic radiation resistance. These factors contribute to a 5-year survival rate of less than 11.5% for patients with loco-regionalized pancreatic tumors.

Advances in conformal techniques using stereotactic body radiotherapy have sought to increase the single dose fractions up to 24 Gy. However, multiple clinical trials have found that large numbers of patients begin to present with grade 3 toxicities as fractions exceed 15 Gy, including bleeding and bowel perforation and, thus far, these high dose treatments with EBRT have resulted in modest gains in local tumor control and negligible improvement in overall survival. Brachytherapy, a version of radiotherapy where the radioactive source is placed inside the tumor to maximize local dose absorption, may also be used. Current brachytherapy techniques deliver gamma-emitting isotopes in temporary catheters or permanent, low-dose seeds. However, none of the brachytherapy modalities improve the clinical outcomes for pancreatic cancer. Therefore, there remains a need for new compositions and treatment methods that can overcome the disadvantages of convention solid tumor treatment and result in tumor growth inhibitions and regression.

### BRIEF SUMMARY OF THE INVENTION

In one aspect, the disclosure provides compositions comprising a first collection of self-assembling conjugates having at least one radionuclide coupled to a first elastin-like polypeptide and a chemotherapeutic.

In another aspect, the disclosure provides a method of killing multiple cancer cells comprising contacting multiple cancer cells with the compositions disclosed herein.

In another aspect, the disclosure provides a method of treating cancer comprising administering to a subject in need thereof a therapeutically effective amount of the compositions disclosed herein.

In another aspect, the disclosure provides a method of treating cancer in a subject in need thereof, the method comprising co-administration of a therapeutically effective amount of a composition comprising a first collection of self-assembling conjugates comprising at least one radionuclide coupled to a first elastin-like polypeptide and a therapeutically effective amount of chemotherapeutic.

### BRIEF DESCRIPTIONS OF THE DRAWINGS

FIG. 1A and FIG. 1B are SDS-PAGE gels showing ITC purification results. Depot-forming ELP after 4 ITC cycles, run at two gel loading concentrations, verify the high purity of ELP (FIG. 1A). Gel showing the relative purify of the micelle-forming ELP throughout each step of the ITC process (FIG. 1B). HS: Hot spin, and CS: Cold spin. Conditions for HS and CS are described in the text below.

FIG. 2 shows a graph of optical density scans for the depot-forming ELP at 350 nm (OD350 nm) as a function of solution temperature.

FIG. 3A and FIG. 3B show HPLC elution traces of CP-PTX conjugation mixtures during the final purification process. Relative purity of the mixture after two rounds of centrifugal ultrafiltration, showing a large proportion of soluble paclitaxel in the product (FIG. 3A). Purity of final product after subsequent washes reduced the level of free paclitaxel to trace amounts (FIG. 3B).

FIG. 4A, FIG. 4B, FIG. 4C, FIG. 4D, FIG. 4E, FIG. 4F, FIG. 4G and FIG. 4H are graphs showing the therapeutic efficacy of brachytherapy and paclitaxel (PTX) in a BxPc3-luc2 pancreatic cancer model. Paclitaxel and CP-PTX exhibited nanomolar in vitro cytotoxicity (FIG. 4A). Intravenous (i.v.) CP-PTX proved more effective than intratumoral (i.t.) chemotherapy for treating orthotopic tumors in combination with ¹³¹I-ELP brachytherapy at a radioactivity dose of 1.5 μCi/mm³ (FIG. 4B). The effect of ¹³¹I-ELP dose was next explored in combination with a constant i.v. CP-PTX dose of 25 mg/kg (FIG. 4C). Subcutaneous tumor regression improved as the radioactivity was increased from 3.3 μCi/mm³, 6.6 μCi/mm³, to 10.0 μCi/mm³ (p<0.001, 2-way ANOVA). Overall survival also improved with increasing radioactivity dose (FIG. 4D, p<0.001, Mantel Cox log-rank). Escalation of the CP-PTX dose with a fixed ¹³¹I-ELP dose showed no effect on tumor response (FIG. 4E, p>0.05, 2-way ANOVA). Survival benefit was likewise insignificant (p=0.1548, Mantel-Cox log-rank), although the 12.5 mg/kg dose trended towards significance (FIG. 4F). When a 2nd injection of CP-PTX at 25 mg/kg was given after 7 d, the duration of the tumor response increased significantly (FIG. 4G, p<0.001, 2-way ANOVA), although survival benefits (FIG. 4H) remained insignificant (p>0.05, Mantel-Cox log-rank).

FIG. 5A, FIG. 5B and FIG. 5C show in vitro cytotoxicity determined by MTS assays for cells treated with a range of concentration of paclitaxel and CP-PTX nanoparticles: MIA PaCa-2 pancreatic cells (FIG. 5A), and AsPc-1 pancreatic cells (FIG. 5B). The dose-response curves were evaluated to determine the relative EC50 values and absolute IC50 values (FIG. 5C). All cell lines demonstrated a reduction of cytotoxicity in EC50 when paclitaxel was formulated as a CP-PTX nanoparticle.

FIG. 6 shows a pilot study in which orthotopic pancreatic BxPc3-luc2 tumors were locally treated with ¹³¹I-ELP brachytherapy and combined with either CP-PTX administered intravenously (i.v.) or co-injected intratumorally (i.t.). Orthotopic tumors were tracked luminescently over the course of 12 days to evaluate response to the different treatments.

FIG. 7 shows Kaplan-Meier survival curves from the radiation dose escalation study in a s.c. BxPc3-luc2 pancreatic xenograft in athymic nude mice. Various doses of ¹³¹I-ELP combined were combined with i.v. infusion of CP-PTX at 25 mg/kg PTX equivalent, resulting in significantly enhanced survival (p<0.0001, Mantel-Cox log-rank test). Within the combination therapy groups, the high-dose brachytherapy group was found to significantly outperform the medium and low dose groups (p<0.05, log-rank).

FIG. 8 shows changes in body weight after treatment as a sign of acute toxicity. An initial drop was seen after surgery, but no treatment regimen showed any statistical difference from the untreated subcutaneous BxPc3-luc2 tumor group.

FIG. 9A and FIG. 9B show the assessment of ¹³¹I-ELP brachytherapy stability in the radiation dose escalation study evaluated by whole body radioactivity measurements (FIG. 9A) of the various ¹³¹I-ELP depots compared to the theoretical profile for ¹³¹Iodine and calculations of depot retention by accounting for isotope decay (FIG. 9B). There was no significant difference in depot stability across treatment groups.

FIG. 10 shows statistical survival assessment using the Kaplan-Meier analysis of the CP-PTX dose escalation study. Combination therapy proved statistically significant over paclitaxel only groups (p<0.05, Mantel-Cox log-rank test). However, no survival advantage was observed at the different paclitaxel doses (p=0.1548).

FIG. 11 shows mouse body weight over the course of treatment as a surrogate marker for acute toxicity onset. No statistical differences were observed between the different CP-PTX doses, minimizing concerns of side effects.

FIG. 12A and FIG. 12B show whole body radioactivity of mice measured over time to track the depot decay profile (FIG. 12A) as compared to the physical half-life of ¹³¹Iodine and calculate the retention of biological retention of the ¹³¹I-ELP depot (FIG. 12B). Results showed no significant difference between any treatment group (p=0.3675) and satisfactory stability (>75% over 21 days).

FIG. 13A, FIG. 13B, FIG. 13C and FIG. 13D are line graphs showing the anti-tumor efficacy of combination therapy using current clinical standards of care in BxPc3-luc2 subcutaneous xenografts. Paclitaxel was delivered as four doses of Abraxane at 12.5 mg/kg of paclitaxel equivalent, once weekly, in combination with 10 μCi/mm³ of ¹³¹I-ELP. 100% complete tumor regression and significantly enhanced median survival were observed (FIG. 13A, FIG. 13B). EBRT (25 Gy in 5 fractions) was delivered in combination with 12.5 mg/kg CP-PTX, but no advantage over monotherapy was observed (FIG. 13C and FIG. 13D). (p=0.5878, 2-way ANOVA). EBRT combination therapy only accomplished tumor growth inhibition and a modest survival benefit. All data represented as mean±SEM. *p<0.05, Mantel-Cox log-rank test.

FIG. 14 shows mouse body weight monitored over the course of treatment as a surrogate marker for acute toxicity in a subcutaneous BxPc3-luc tumor model. No statistical differences were observed between the Abraxane, ¹³¹I-ELP+Abraxane, and untreated mice.

FIG. 15A and FIG. 15B show whole body radioactivity of mice tracked over time to ensure that the effects of Abraxane did not result in a decay profile (FIG. 15A) that differed considerably from the physical half-life of ¹³¹Iodine and negatively affected the ¹³¹Iodine retention of the intratumoral ELP depot (FIG. 15B). The results proved similar to the previous combination studies.

FIG. 16 shows the hypofractionated dose schedule for the EBRT study, wherein tumors were treated with cumulative dose of 25 Gy X-ray radiation delivered as five doses at 5 Gy.

FIG. 17 shows body weight changes after treatment to look for signs of acute toxicity. No statistical differences were observed between the X-ray combination therapy, X-ray only, CP-PTX only, and untreated groups.

FIG. 18A, FIG. 18B, FIG. 18C and FIG. 18D are Bliss Independence isobolograms constructed to evaluate if combination therapy responses were synergistic or merely the result of additive effects. Trials analyzed included: ¹³¹I-ELP with single i.v. dose of CP-PTX (FIG. 18A), ¹³¹I-ELP with two i.v. doses of CP-PTX (FIG. 18B), ¹³¹I-ELP with four i.v. doses of Abraxane (FIG. 18C, Nab), and EBRT with four i.v. doses of CP-PTX (FIG. 18D). Mathematical synergy is indicated when the actual tumor regression (solid line) is lower than the Bliss prediction (dashed line) and exceeds the 95% confidence interval (shaded). All data are shown as mean f SEM.

FIG. 19A, FIG. 19B, FIG. 19C, FIG. 19D, FIG. 19E, FIG. 19F, FIG. 19G, FIG. 19H, FIG. 19I, and FIG. 19J show that the temporal coordination of radiation delivery with the sensitization effects of paclitaxel achieved potent synergy. Paclitaxel treated cells, regardless of vehicle, show temporal variation in arresting cells in the targeted late G2/M phase (FIG. 19A). While the dose rate of ¹³¹I-ELP is lower than EBRT sources, the continuous exposure ensures cells are irradiated as they enter the G2/M phase, unlike once-a-day EBRT (FIG. 19B). Moreover, the cumulative radiation of ¹³¹I-ELP achieves a significantly higher dose (FIG. 19C). TUNEL IHC staining revealed how these factors combine (FIG. 19D) to induce greater areas of apoptosis in BxPc3 tumors after 12 days. Samples were compared from: untreated (FIG. 19E), CP-PTX only (FIG. 19F), EBRT only (FIG. 19G), ¹³¹I-ELP only (FIG. 19H), EBRT combination therapy (FIG. 19I), and ¹³¹I-ELP combination therapy treated tumors (FIG. 19J). *p>0.05.

FIG. 20A, FIG. 20B, FIG. 20C, FIG. 20D, FIG. 20E, FIG. 20F and FIG. 20G show the immunohistochemistry (IHC) of orthotopic BxPc3-luc2 tumors after 12 days of treatment that revealed differential effects of radiation on the underlying tumor microenvironment. ¹³¹I-ELP was administered at 10 μCi/mm³, EBRT at 5×5 Gy, and CP-PTX at 12.5 mg/kg, once weekly. The IHC markers H&E staining (FIG. 20A), Claudin-4 (FIG. 20B), CD-31 (PECAM-1) (FIG. 20C), and CD-144 (VE-Cadherin) (FIG. 20D) were examined as molecular markers of cellular adhesion and interstitial permeability. Disruption of junction proteins was observed in regions of tumor tissue proximal to ¹³¹I-ELP depots but was not observed for tumors treated with EBRT. In vivo imaging studies were conducted using fluorescently-labeled CP-PTX, administered once-weekly at 12.5 mg/kg over 10 days, with either EBRT (25 Gy) or ¹³¹I-ELP brachytherapy (10 μCi/mm³) in a hind-flank tumor model. Drug accumulation in the tumor was monitored over time (FIG. 20E and FIG. 20F) and total exposure was quantified as area under the curve (FIG. 20G, AUC). ¹³¹I-ELP treatment induced significantly higher CP-PTX tumor uptake than EBRT (*p<0.001, ANOVA). Data is represented as the mean±SEM.

FIG. 21A and FIG. 21B show the light scattering analysis of CP-PTX particles after sulfoCy5.5 labeling. DLS showed that fluorescently labeled nanoparticles retained their size with only a slight increase in the RH (FIG. 21A). SLS data indicate a higher RG, indicating that the nanoparticles may no longer be spherical (FIG. 21B). However, fluorescent emissions significantly confounded the accuracy of the SLS analysis due to spectral overlap with the excitation laser.

FIG. 22A and FIG. 22B show raw data results from sulfoCy5.5-CP-PTX uptake experiment. Raw fluorescent flux readings were quantified from each mouse tumor after receiving 110 μL injections at 0 d and 7 d (FIG. 22A). Tumor growth was also tracked over time, as the different growth profiles could influence the amount of drug capable of accumulating in the tumor (FIG. 22B). ¹³¹I-ELP tumors remained significantly smaller than the other groups (p=0.0123).

FIG. 23A and FIG. 23B show Alexa690 maleimide conjugated to the hydrophilic, chimeric ELP (CPELP) without any paclitaxel. DLS analysis showed that resulting particle did not self-assemble at the same scale as CP-PTX. Instead it is near the upper range of single biopolymer strand size (FIG. 23A). Partial Zimm plot analysis with SLS revealed a higher mass-weighted radius of gyration (RG). This indicated that polymers only self-assembled in associations of 6 or less and aren't real nanoparticles (FIG. 23B).

FIG. 24A and FIG. 24B show a fluorescent uptake study where the effects of paclitaxel are removed. Chimeric ELP polymers were conjugated to the fluorophore Alexa680 maleimide. Normalized fluorescent analysis of tumors receiving either ¹³¹I-ELP, EBRT, ELP only depots, or are left untreated (FIG. 24A). Significantly higher flux is seen in brachytherapy treated tumors. Area under the curve analysis reveals that ¹³¹I-ELP produces 1.7-fold higher drug accumulation compared to EBRT (FIG. 24B). When compared to ELP sham injections or untreated tumors, this increases to a 2.3-fold higher AUC accumulation level.

FIG. 25A and FIG. 25B show light scattering analysis of CP-Cy5.5 conjugates. DLS showed that the conjugates primarily self-assembled into nanoparticles with an effective hydrodynamic radius of 46.7 nm (FIG. 25A). SLS showed that the mass-weighted radius of gyration was 51.8 nm, giving it a slightly elongated form factor compared to CP-PTX but still considered reasonable approximation (FIG. 25B).

FIG. 26A and FIG. 26B show fluorescent accumulation analysis of treated tumors using a CP-Cy5.5 nanoparticle. Fluorescent flux in ¹³¹I-ELP treated tumors was significantly higher than untreated tumors (FIG. 26A, p=0.006, 2-way ANOVA). Area under the curve analysis revealed a 1.6-fold increase in total accumulation of CP-Cy5.5, as measured by fluorescent flux (FIG. 26B).

FIG. 27A, FIG. 27B, FIG. 27C, FIG. 27D, FIG. 27E and FIG. 27F are graphs showing the efficacy of optimized ¹³¹I-ELP brachytherapy and i.v. CP-PTX chemotherapy in multiple pancreatic tumor types and models. Combination therapy achieved 100% complete regression in MIA PaCa-2 subcutaneous xenografts (FIG. 27A) with survival extended 2.8-fold to 92 d (FIG. 27B, *p<0.05, Mantel Cox log rank test). A subcutaneous AsPc-1 tumor model also demonstrated a 100% overall response rate to treatment (FIG. 27C); however, only 28.6% experienced complete regression, with survival extended 2.2-fold to 99 days compared to controls (FIG. 27D, *p<0.05, Mantel Cox log rank test). Bioluminescent tracking of orthotopic BxPc-luc2 tumors after combination therapy (FIG. 27E) revealed an 83.3% complete response rate with a median survival increase by 3.7-fold over untreated tumors to 58 d (FIG. 27F, *p<0.05, log-rank Mantel Cox). Data represented as mean±SEM.

FIG. 28A and FIG. 28B show the finalized combination therapy regimen (10.0 μCi/mg ¹³¹I-ELP with 12.5 mg/kg CP-PTX q.w. for 4 weeks) used to treat subcutaneous MIA PaCa-2 pancreatic tumor xenografts. A spaghetti plot of individual tumor responses treatment consistently resulted in complete regression (FIG. 28A). Body weight monitoring showed no signs of acute toxicity in any of the animals (FIG. 28B).

FIG. 29A and FIG. 29B show the radiation stability of the ELP biopolymer depot monitored by tracking whole body activity over time (FIG. 29A) and calculating the percent depot retention by decay correcting the retained activity in each mouse (FIG. 29B).

FIG. 30A, FIG. 30B, FIG. 30C and FIG. 30D show mice xenografted with AsPc-1 subcutaneous tumors and then treated with variable ¹³¹I-ELP doses and four, once weekly injections of 12.5 mg/kg CP-PTX. Individual tumor regressions are shown for 10.0 μCi/mg treated mice (FIG. 30A), 6.6 μCi/mg treated mice (FIG. 30B), and untreated mice (FIG. 30C). Body weight was also tracked, showing no signs of acute toxicity in any group (FIG. 30D).

FIG. 31A and FIG. 31B show the radiation stability of the ELP biopolymer depot monitored by tracking whole body activity over time (FIG. 31A) and the percent depot retention was calculated by decay in each mouse (FIG. 31B).

FIG. 32A and FIG. 32B show regression plots of tumor responses in an orthotopic BxPc3-luc2 tumor model. ¹³¹I-ELP brachytherapy was at 8.83 μCi/mm3 with four weekly doses of 12.5 mg/kg CP-PTX (FIG. 32A). Significant regression was observed compared to monotherapy controls (p<0.05, ANOVA). In fact, all tumors showed partial regression with 5/6 achieving complete regression. ¹³¹I-ELP brachytherapy was given with two weekly doses of 25 mg/kg CP-PTX (FIG. 32B). While this regression was similarly significant against monotherapy controls (p<0.05, ANOVA), the response showed no significant difference from the 12.5×4 combination therapy group.

FIG. 33A, FIG. 33B, and FIG. 33C show the toxicity assessment of ¹³¹I-ELP combination therapy in BxPc3 tumors grafted orthotopically in the pancreas. Body weight was tracked over time post-treatment and demonstrated no adverse morbidity other than weight loss associated with surgery at the time of treatment (FIG. 33A). Serological α-amylase levels were measured to assay acute inflammation and damage to the healthy pancreas as a result of continuous ¹³¹I-ELP irradiation (FIG. 33B). Levels in the treated group were not significantly different from healthy, control mice. A serial biodistribution study examined accumulation of ¹³¹Iodine in off-target tissues (FIG. 33C). Activities remained 3-5 orders of magnitude below the therapeutic dose, and the associated cumulative exposure was less than 1 Gy. Data represented as mean±SEM.

FIG. 34A and FIG. 34B show the radiation profile of the radioactive depot was monitored in the orthotopic BxPc3-luc 2 models by tracking whole body activity over time (FIG. 34A) and the percent depot retention was calculated by decay in each mouse (FIG. 34B).

FIG. 35 shows a table summarizing the data from tumor responses to radiation combination therapy.

FIG. 36 shows a table summarizing the data from tumor responses to single agent therapy.

FIG. 37A and FIG. 37B show the nanoparticle size and shape analysis of CP-PTX. Dynamic light scattering was utilized to determine that CP-PTX formed highly monodisperse micelle populations with a hydrodynamic radius (RH) of 39.4 nm (FIG. 37A). A small unimer population was detected. Static light scattering provided the radius of gyration (RG), aggregate particle weight, polymer packing, and shape factor (FIG. 37B).

FIG. 38A, FIG. 38B, FIG. 38C, FIG. 38D, FIG. 38E, FIG. 38F and FIG. 38G show Hematoxylin and Eosin histology images of tissue specimens representing normal murine pancreas (FIG. 38A), untreated BxPc3-luc2 tumors (FIG. 38B), CP-PTX treated tumors (FIG. 38C), 25 Gy EBRT monotherapy (FIG. 38D), ¹³¹I-ELP monotherapy (FIG. 38E), EBRT combination therapy (FIG. 38F), and ¹³¹I-ELP combination therapy (FIG. 38G) treated tumors. Full panels show representative pathological patterning, while insets emphasize cellular characteristics.

FIG. 39A and FIG. 39B show visual inspection of the H&E specimens that revealed regions of cellular apoptosis. The estimated percentage of cellular apoptosis (FIG. 39A and size of these regions (FIG. 39B) showed differential effects to the BxPc3-luc2 tumors which could be attributed to differential treatment methods.

FIG. 40 shows TUNEL staining of a healthy mouse pancreas.

FIG. 41A, FIG. 41B, FIG. 41C, FIG. 41D, FIG. 41E and FIG. 41F show differential TUNEL IHC staining of BxPc3-luc2 tumors when treated with different conditions: untreated (FIG. 41A), EBRT only (FIG. 41B), ¹³¹I-ELP only (FIG. 41C), CP-PTX only (FIG. 41D), EBRT with CP-PTX (FIG. 41E), and ¹³¹I-ELP with CP-PTX (FIG. 41F). Insets show the staining of the entire tumor specimen. ¹³¹I-ELP combination therapy shows an intense level of TUNEL staining not found across any other treatment group. Insets show stitched images of the entire tumor cross-section.

FIG. 42 shows the coverage of TUNEL staining quantified relative to the tumor area for all treatment samples. Therapy consisting of ¹³¹I-ELP with CP-PTX was found to have a significantly higher proportion of tumor apoptosis (p<0.05).

FIG. 43A and FIG. 43B show the immunohistological staining of Claudin-4 tight junction protein in a normal murine pancreatic tissue (FIG. 43A) and an untreated BxPc3-luc2 tumor xenograft specimen (FIG. 43B).

FIG. 44A, FIG. 44B, FIG. 44C, FIG. 44D, FIG. 44E show Claudin-4 expression in BxPc3-luc2 xenografts after treatment with CP-PTX only (FIG. 44A), EBRT (25 Gy) only (FIG. 44B), ¹³¹I-ELP only (FIG. 44C), EBRT combination therapy with CP-PTX (FIG. 44D), and ¹³¹I-ELP combination therapy (FIG. 44E).

FIG. 45A and FIG. 45B show pathological analysis of Claudin-4 quality and quantity in BxPc3-luc2 tumors. First, the intensity of the Claudin-4 staining was quantified (FIG. 45A): 3-intense, 2-moderate, 1-light, and 0-no staining. Next, the relative area of Claudin-4 coverage was quantified by converting to a binary mask (FIG. 45B). Significant (p<0.05) reduction in staining was observed for CP-PTX, ¹³¹I-ELP monotherapy, and ¹³¹I-ELP combination therapy treatments.

FIG. 46A and FIG. 46B shows CD-31 staining of a normal murine prostate (FIG. 46A) and an untreated BxPc3-luc2 tumor (FIG. 46B). Normal tissue shows luminal staining of vessels. Tumor xenografts, however, show light expression in the stroma.

FIG. 47A, FIG. 47B, FIG. 47C, FIG. 47D and FIG. 47E show PECAM-1 expression via CD31 IHC staining in BxPc3-luc2 xenografts after receiving different treatments: CP-PTX only (FIG. 47A), EBRT (25 Gy) only (FIG. 47B), ¹³¹I-ELP only (FIG. 47C), EBRT combination therapy with CP-PTX (FIG. 47D), and ¹³¹I-ELP combination therapy (FIG. 47E). No significant difference in expression was observed amongst treatments.

FIG. 48A and FIG. 48B show pathological analysis of PECAM-1 (CD31) in BxPc3-luc2 tumors after treatment. The qualitative intensity of CD-31 staining in cell cytoplasm was first evaluated, with no differences observed (FIG. 48A). The relative coverage area of CD-31 staining also showed comparable expression amongst all treatment groups (FIG. 48B).

FIG. 49A and FIG. 49B show immunohistological staining of VE-Cadherin adherens junctions, using CD144, in normal murine pancreatic tissue (FIG. 49A) and an untreated BxPc3-luc2 tumor xenograft specimen (FIG. 49B).

FIG. 50A and FIG. 50B show tumor tissue CD-144 histology scores (H-Score) that combined the intensity and frequency of nuclear staining within cells. Results showed a significant reduction in VE-Cadherin for ¹³¹I-ELP monotherapy over untreated tumor specimens (FIG. 50A) (p<0.05). Area of expression showed a trend of reduced VE-Cadherin expression for ¹³¹I-ELP treatment groups but was not statistically significant (FIG. 50B).

FIG. 51A, FIG. 51B, FIG. 51C, FIG. 51D and FIG. 51E show differential effects of treatment on VE-Cadherin immunohistology in a BxPc3-luc2 tumor xenograft. Treatment groups were comprised of CP-PTX (FIG. 51A), X-ray EBRT (FIG. 51B), ¹³¹I-ELP (FIG. 51C), EBRT combination therapy (FIG. 51D), and ¹³¹I-ELP combination therapy (FIG. 51E). Tumors were examined 12 days after initiating treatment.

FIG. 52A and FIG. 52B show Masson trichrome staining for detection of collagen (cyan), cytoplasm (pink), and nuclei (purple). Tissue samples represented are taken from normal murine pancreatic tissue (FIG. 52A) and an untreated BxPc3-luc2 tumor xenograft specimen (FIG. 52B). Insets emphasize cellular features.

FIG. 53A, FIG. 53B, FIG. 53C, FIG. 53D, and FIG. 53E show effects of different treatments on the tumor stromal collagen microenvironment, as indicated by Masson trichrome staining (collagen=cyan, cytoplasm=pink, and nuclei=purple). Representative tissue samples are shown for CP-PTX monotherapy (FIG. 53A), EBRT monotherapy (FIG. 53B), ¹³¹I-ELP monotherapy (FIG. 53C), EBRT combination therapy (FIG. 53D), and ¹³¹I-ELP combination therapy (FIG. 53E). Tumor samples were collected 12 days after treatment.

FIG. 54 shows the abundance and quality of stromal collagen assessed in a blind, randomized pathology reading. Ranks consisted of 0=normal collagen, 1=minimal stroma, 2=light stroma, 3=moderate stroma, 4=dense stroma. External beam radiation was found to consistently induce a dense phenotype while ¹³¹I-ELP combination therapy produced a significant reduction of stromal collagen into the light-moderate range (p<0.05).

FIG. 55 shows an H&E specimen prepared from mouse #1016532-85. No residual tumor tissue was noted. Normal pancreatic tissue and spleen were noted. There was one foci of inflammation with a germinal center—possibly a granuloma. Tissues also displayed a normal level of collagen, Claudin-4, CD-31, and CD-144 expression consistent with a healthy pancreas.

FIG. 56 shows an H&E specimen prepared from mouse #1016532-81. Minimal residual tumor tissue was observed with a large foci of necrosis and histiocytic cells. Normal pancreatic tissue and spleen were noted. A moderate amount of stromal collagen still persisted. Normal levels of collagen, Claudin-4, CD-31, and CD-144 levels were consistent with a healthy pancreas.

### DETAILED DESCRIPTION OF THE INVENTION

Disclosed herein are compositions for liquidly injectable, self-stabilizing biopolymers for the delivery of radionuclide brachytherapy and their methods of use. The compositions comprise a collection of self-assembling conjugates comprising at least one radionuclide coupled to a first elastin-like polypeptide which forms an intratumoral deposition that irradiates the tumor from the inside-out. When combined with systemic or local chemotherapy, the treatment methods can overcome the intrinsic resistance of pancreatic tumors and result in tumor regression.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the terms “administering,” “providing” and “introducing” are used interchangeably herein and refer to the placement of the compositions of the disclosure into a subject by a method or route which results in at least partial localization of the composition to a desired site. The compositions can be administered by any appropriate route which results in delivery to a desired location in the subject.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

As used herein, the term “chemotherapeutic” or “anti-cancer drug” includes any drug used in cancer treatment or any radiation sensitizing agent. Chemotherapeutics may include alkylating agents (including, but not limited to, cyclophosphamide, mechlorethamine, chlorambucil, melphalan, dacarbazine, nitrosoureas, and temozolomide), anthracyclines (including, but not limited to, daunorubicin, doxorubicin, epirubicin, idarubicin, mitoxantrone, and valrubicin), cytoskeletal disruptors or taxanes (including, but not limited to, paclitaxel, docetaxel, abraxane, and taxotere), epothilones, histone deacetylase inhibitors (including, but not limited to, vorinostat and romidepsin), topoisomerase inhibitors (including, but not limited to, irinotecan, topotecan, etoposide, tenoposide, and tafluposide), kinase inhibitors (including, but not limited to, bortezomib, erlotinib, gefitinib, imantinib, vemurafenib, and vismodegib), nucleotide analogs and precursor analogs (including, but not limited to, azacitidine, azathioprine, capecitabine, cytarabine, doxifluridine, fluorouracil, gemcitabine, hydroxyurea, mercaptopurine, methotrexate, and tioguanine), peptide antibiotics (including, but not limited to, bleomycin and actinomycin), platinum-based agents (including, but not limited to, carboplatin, cisplatin and oxaliplatin), retinoids (including, but not limited to, tretinoin, alitretinoin, and bexarotene), vinca alkaloids and derivatives (including, but not limited to, vinblastine, vincristine, vindesine, and vinorelbine), or combinations thereof. The chemotherapeutic may in any form necessary for efficacious administration and functionality. For example, the chemotherapeutic may be bound to a peptide or protein, such as Abraxane, an albumin-bound paclitaxel.

As used herein, the term “critical micelle temperature” or “CMT” defines the temperature at with a micelle will form. Below the CMT, micelles will not form.

The terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a composition as disclosed herein that may provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the regenerative cells, the type or extent of supplemental therapy used, ongoing disease process and type of treatment desired (e.g., aggressive vs. conventional treatment).

As used herein, the term “micelle” refers to an organized auto assembly of molecules formed in a liquid where the hydrophilic regions are in contact with the surrounding solvent and the hydrophobic regions are sequestered in the center or core of the micelle. In some embodiments, the micelle may be a nanoparticle.

As used herein, the term “nanoparticle” refers to a particle with at least one dimension less than about 100 nm. Nanoparticles include, but are not limited to, nanopowders, nanoclusters, nanocrystals, and micelles.

A “peptide” or “polypeptide” is a linked sequence of two or more amino acids linked by peptide bonds. The polypeptide can be natural, synthetic, or a modification or combination of natural and synthetic. Domains are portions of a polypeptide or protein that form a compact unit and are typically 15 to 350 amino acids long.

As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition, and/or decreasing the risk of developing pathology associated with the infection, the disease, disorder, and/or condition.

“Radionuclide,” “radioactive nuclide,” “radioisotope,” or “radioactive isotope” are used interchangeably herein to represent any atom that has excess nuclear energy and is, therefore, unstable. The excess energy may be emitted as gamma, alpha, beta, or a combination thereof. The radionuclide may provide irradiation through beta-particles, alpha-particles or Auger electrons.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

As used herein, the term “transition temperature” or “Tt” refers to the temperature at which the material changes from one state to another, for example, soluble to insoluble. For example, below the Tₜ the conjugate may be highly soluble. Upon heating above the transition temperature, for example, the conjugate may aggregate, forming a separate phase.

As used herein, “treat,” “treating” and the like mean a slowing, stopping or reversing of progression of a disease or disorder when provided a composition described herein to an appropriate control subject. The terms also mean a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve or affect the disease or symptoms of the disease.

### 2. Compositions

Provided herein are compositions comprising a first collection of self-assembling conjugates comprising at least one radionuclide coupled to a first elastin-like polypeptide and a chemotherapeutic.

a) Radionuclide Coupled Elastin-Like Polypeptide

The first collection of self-assembling conjugates may include a first elastin-like polypeptide. Elastin-like polypeptides (ELP) are thermally responsive polypeptides. “ELP” refers to a polypeptide comprising the pentapeptide repeat sequence (VPGX¹G)ₚ (SEQ ID NO: 3), wherein X¹ is any amino acid and n is an integer greater than or equal to 1.

The first elastin-like polypeptide may comprise an amino acid sequence of (VPGX¹G)ₚ (SEQ ID NO: 3), wherein X¹ is any amino acid, p is 1 to 500 and wherein the elastin-like polypeptide has a transition temperature below about 42° C. In some embodiments, p is an integer from 1 to 500, from 1 to 400, from 1 to 300, from 1 to 200, from 1 to 100, from 1 to 50, from 50 to 500, from 50 to 400, from 50 to 300, from 50 to 200, from 50 to 100, from 100 to 500, from 100 to 400, from 100 to 300, from 100 to 200, from 200 to 500, from 200 to 400, from 200 to 300, from 200 to 500, from 300 to 500, from 300 to 400, or from 400 to 500.

The first elastin-like polypeptide may comprise an amino acid sequence of (VPGX²G)_{q}(GᵣY)ₛ (SEQ ID NO: 4), wherein X² is any amino acid, q is 1 to 500, r is 0 to 10, and s is 1-250 and wherein the elastin-like polypeptide has a transition temperature below about 42° C. In some embodiments, q is an integer from 1 to 500, from 1 to 400, from 1 to 300, from 1 to 200, from 1 to 100, from 1 to 50, from 50 to 500, from 50 to 400, from 50 to 300, from 50 to 200, from 50 to 100, from 100 to 500, from 100 to 400, from 100 to 300, from 100 to 200, from 200 to 500, from 200 to 400, from 200 to 300, from 200 to 500, from 300 to 500, from 300 to 400, or from 400 to 500. In some embodiments, r is an integer from 0 to 10, from 0 to 9, from 0 to 8, from 0 to 7, from 0 to 6, from 0 to 5, from 0 to 4, from 0 to 3, from 0 to 2, from 0 to 1, from 1 to 10, from 1 to 9, from 1 to 8, from 1 to 7, from 1 to 6, from 1 to 5, from 1 to 3, from 1 to 2, from 2 to 10, from 2 to 9, from 2 to 8, from 2 to 7, from 2 to 6, from 2 to 5, from 2 to 4, from 2 to 3, from 3 to 10, from 3 to 9, from 3 to 8, from 3 to 7, from 3 to 6, from 3 to 5, from 3 to 4, from 4 to 10, from 4 to 9, from 4 to 8, from 4 to 7, from 4 to 6, from 4 to 5, from 5 to 10, from 5 to 9, from 5 to 8, from 5 to 7, from 5 to 6, from 6 to 10, from 6 to 9, from 6 to 8, from 6 to 7, from 7 to 10, from 7 to 9, from 7 to 8, from 8 to 10, from 8 to 9, or from 9 to 10. In some embodiments, s is an integer from 1 to 250, from 1 to 200, from 1 to 150, from 1 to 100, from 1 to 50, from 50 to 250, from 50 to 200, from 50 to 150, from 50 to 100, from 100 to 250, from 100 to 200, from 100 to 150, from 150 to 250, from 150 to 200, or from 200 to 250.

The first elastin-like polypeptide conjugated to the radionuclide may have phase transition behavior. Phase transition may refer to aggregation, which may occur sharply and in some instances reversibly at or above the transition temperature. The Tₜ can be adjusted by varying the amino acid sequence of the elastin-like polypeptide, by varying the length of the polypeptide, or a combination thereof. The transition temperature may be below about 45° C., about 40° C., about 35° C., about 30° C., about 25° C., about 20° C., or about 15° C. The transition temperature may be below about 42° C.

In certain embodiments, the first elastin-like polypeptide comprises an amino acid sequence of (VPGVG)ₘ(GY)ₙ (SEQ ID NO: 1), wherein m is 50-250 and n is 1 to 50. In some embodiments, m is an integer from 50 to 200, from 50 to 150, from 50 to 100, from 100 to 250, from 100 to 200, from 100 to 150, from 150 to 200, from 150 to 200, or from 200 to 250. In some embodiments, m is 120. In some embodiments, n is an integer from 1 to 50, from 10 to 50, from 20 to 50, from 30 to 50, from 40 to 50, from 1 to 10, from 1 to 20, from 1 to 30 or from 1 to 40. In some embodiments, n is 7. In exemplary embodiments, m is 120 and n is 7.

The collection of self-assembling conjugates may include varying amounts of elastin-like polypeptide chains. For example, the assembly of self-assembling conjugates may include about 10 to about 200 elastin-like polypeptide chains per collection, such as about 10 to about 100 or about 50 to about 200.

The radionuclide may be any of the radioisotopes known in the art capable of providing irradiation through beta-particles, alpha-particles, gamma rays or Auger electrons. The radionuclide may include but is not limited to ¹³¹Cesium, ¹³⁷Cesium, ⁶⁰Cobalt, ¹⁹²Iridium, ¹²⁵Iodine, ¹³¹Iodine, ¹⁰³Palladium, ¹⁰⁶Ruthenium, ²²³Radium, ²²⁶Radium, ⁹⁰Yttrium, ¹⁷⁷Lutetium, ¹¹¹Indium, ¹⁸⁶Rhenium, ⁸⁹Strontium, ¹⁵³Samarium, ³²Phosphorous, ²²⁵Actinium, ²¹¹Astatine, ²¹³Bismuth, and ²¹²Lead. In some embodiments, the radionuclide is ¹³¹Iodine.

Each of the first elastin-like polypeptides may be coupled with at least one radionuclide. In some embodiments, each of the first elastin-like polypeptides may be coupled with at least one, at least two, at least three, at least four, at least five, at least six, at least seven, at least eight, at least nine, or at least ten radionuclides.

The first collection of self-assembling conjugates may individually self-assemble into a variety of shapes and sizes. In some embodiments, first collection of self-assembling conjugates may individually self-assemble into a micelle. The micelles may be may be rod-shaped or spherical, or the collection may include combinations of differently shaped nanoparticles.

The elastin-like polypeptides conjugated to a radionuclide may be defined by a critical micelle temperature. This is the minimal temperature at which micelles will form. Below the critical micelle temperature, micelles will not form. The critical micelle temperature may be below about 40° C., about 35° C., about 30° C., about 25° C., about 20° C. or about 15° C. In some embodiments the critical micelle temperature is below about 23° C.

The micelles may have a varying average hydrodynamic radius. In some embodiments, the nanoparticles have an average hydrodynamic radius of about 10 nm to about 100 nm, such as about 25 nm to about 75 nm or about 40 nm to about 60 nm. In some embodiments, the nanoparticle may have an average hydrodynamic radius of greater than 10 nm, greater than 20 nm, greater than 30 nm, greater than 40 nm, or greater than 50 nm. In some embodiments, the nanoparticle may have an average hydrodynamic radius of less than 100 nm, less than 90 nm, less than 80 nm, less than 70 nm, less than 60 nm, or less than 50 nm.

The micelle may also be described by its average radius of gyration. For example, the nanoparticle may have an average radius of gyration of about 10 nm to about 100 nm, such as about 25 nm to about 75 nm or about 40 nm to about 60 nm. In some embodiments, the nanoparticle may have an average radius of gyration of greater than 10 nm, greater than 20 nm, greater than 30 nm, greater than 40 nm, or greater than 50 nm. In some embodiments, the nanoparticle may have an average radius of gyration of less than 100 nm, less than 90 nm, less than 80 nm, less than 70 nm, less than 60 nm, or less than 50 nm.

The first elastin-like polypeptide conjugated to the radionuclide and the micelles they form may have phase transition behavior, wherein the micelles coacervate at a transition temperature or the micelle-coacervation transition temperature (Tₜ). Phase transition may refer to the aggregation, which may occur sharply and in some instances reversibly at or above the micelle-coacervation transition temperature. The Tₜ can be adjusted by varying the amino acid sequence of the elastin-like polypeptide, by varying the length of the polypeptide, or a combination thereof.

The micelle-coacervation transition temperature may be below about 60° C., about 55° C., about 50° C., about 45° C., about 40° C., about 35° C., about 30° C., about 25° C., about 20° C., or about 15° C. In some embodiments the micelle-coacervation transition temperature is below about 42° C.

Phase transition behavior may also enable purification of the conjugate using inverse transition cycling, thereby eliminating the need for chromatography. “Inverse transition cycling” refers to a protein purification method for polypeptides having phase transition behavior, and the method may involve the use of the conjugate's reversible phase transition behavior to cycle the solution through soluble and insoluble phases, thereby removing contaminants and eliminating the need for chromatography.

b) Chemotherapeutic

The composition may include a chemotherapeutic. The chemotherapeutic may be chosen from alkylating agents, anthracyclines, cytoskeletal disruptors or taxanes, epothilones, histone deacetylase inhibitors, topoisomerase inhibitors, kinase inhibitors, nucleotide analogs and precursor analogs, peptide antibiotics, platinum-based agents, retinoids, vinca alkaloids and derivatives, or combinations thereof. In some embodiments, the chemotherapeutic is a cytoskeletal disruptor or taxane. In exemplary embodiments, the chemotherapeutic is paclitaxel.

The chemotherapeutic may be contained in a second collection of self-assembling conjugates, wherein the second collection of self-assembling conjugates comprises the chemotherapeutic coupled to a second elastin-like polypeptide. The second elastin-like polypeptide may comprise an amino acid sequence SKGPG(X³GVPG)ₓWPC(GGC)_{z} (SEQ ID NO:2), wherein X³ is an amino acid or a combination of amino acids, x is 40 to 400 and z is 1 to 50. In some embodiments, the second elastin-like polypeptide comprises an amino acid sequence of SKGPG(X³GVPG)ₓWPC(GGC)_{z} (SEQ ID NO:2), wherein X³ is V:G:A in a ratio of 1:7:8. In some embodiments, x is an integer from 40 to 400, from 40 to 300, from 40 to 200, from 40 to 100, from 100 to 200, from 100 to 150, from 100 to 200, from 100 to 300 from 100 to 400, from 200 to 400, from 200 to 300, or from 300 to 400. In some embodiments, x is 160. In some embodiments, z is an integer from 1 to 50, from 10 to 50, from 20 to 50, from 30 to 50, from 40 to 50, from 1 to 10, from 1 to 20, from 1 to 30 or from 1 to 40. In some embodiments, z is 7. In certain embodiments, x is 160 and z is 7

The second collection of self-assembling conjugates may individually self-assemble into a variety of shapes and sizes. In some embodiments, the assembly of self-assembling conjugates may be a nanoparticle. The nanoparticle may be rod-shaped or spherical, or the collection may include combinations of differently shaped nanoparticles. In some embodiments, the nanoparticle is a micelle.

The second collection of self-assembling conjugates may include varying amounts of self-assembling polypeptide chains. For example, the assembly of self-assembling conjugates may include about 10 to about 200 self-assembling conjugates per assembly, such as about 10 to about 100, about 50 to about 200.

The second elastin-like polypeptide may also have phase transition behavior. The transition temperature (Tₜ) can be adjusted by varying the amino acid sequence of the polypeptide, by varying the length of the polypeptide, or a combination thereof. Phase transition may refer to the aggregation, which may occur sharply and in some instances reversibly at or above the transition temperature. The transition temperature may be below about 60° C., about 55° C., about 50° C., about 45° C., about 40° C., about 35° C., about 30° C., about 25° C., about 20° C., or about 15° C.

Phase transition behavior may also enable purification of the conjugate using inverse transition cycling, thereby eliminating the need for chromatography. “Inverse transition cycling” refers to a protein purification method for polypeptides having phase transition behavior, and the method may involve the use of the conjugate's reversible phase transition behavior to cycle the solution through soluble and insoluble phases, thereby removing contaminants and eliminating the need for chromatography.

### 3. Methods of Use

a) Method of Killing Cancer Cells

The present disclosure also provides a method of killing multiple cancer cells. The method may include contacting multiple cancer cells with the composition as detailed herein to the subject. The cancer cells may be in an in vitro environment or an in vivo environment. In some embodiments, the cancer cells are in a subject. Many different types of cancer cells may be killed by chemotherapeutics. The compositions as detailed herein may be used to deliver chemotherapeutics to any cancer cell type.

b) Method of Treating Cancer

The present disclosure also provides methods of treating cancer. One of the methods comprises administering to a subject in need thereof a therapeutically effective amount of the composition as detailed herein to the subject.

The compositions as detailed herein may be used to treat any cancer type or subtype. The cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid or uterus.

In some embodiments, the cancer is a solid tumor. Examples of cancers that are solid tumors include, but are not limited to, pancreatic, bladder, non-small cell lung cancer (NSCLC), breast and ovarian cancers. In some embodiments, the cancer is pancreatic cancer, prostate cancer, or melanoma.

The composition may be administered locally to the cancer, such as intratumoral.

c) Co-Administration Method of Treating Cancer

Another method of treating cancer comprises co-administration of a therapeutically effective amount of a composition comprising a first collection of self-assembling conjugates comprising at least one radionuclide coupled to a first elastin-like polypeptide and a therapeutically effective amount of chemotherapeutic.

The co-administration may be simultaneous, separate or sequential. The co-administration may be in any order, and the components may be administered separately or as a fixed combination. For example, the treatment of cancer according to the invention may comprise administration of the radionuclide composition and administration of the chemotherapeutic, simultaneously or sequentially in any order, in jointly therapeutically effective amounts or effective amounts, e.g. in daily dosages corresponding to the amounts described herein. The individual components can be administered separately at different times during the course of treatment or concurrently in divided or single dosage forms. The instant invention is therefore to be understood as embracing all such regimes of simultaneous or alternating treatment and the term “administering” is to be interpreted accordingly.

The composition and the chemotherapeutic may conveniently be presented in a single dose or as divided doses administered at appropriate intervals, for example, as two, three, four or more sub-doses per day. The sub-dose itself may be further divided, e.g., into a number of discrete loosely spaced administrations. In some embodiments the chemotherapeutic is administered in multiple doses.

The radionuclide composition and the chemotherapeutic may be administered locally to the cancer, such as intratumoral. The chemotherapeutic may be administered systemically, such as enterally or parenterally. The systemic administration may be by injection, intravenously or intraperitoneally, or orally.

The method may be used to treat any cancer type or subtype. The cancer may be a carcinoma, sarcoma, lymphoma, leukemia, melanoma, mesothelioma, multiple myeloma, or seminoma. The cancer may be a cancer of the bladder, blood, bone, brain, breast, cervix, colon/rectum, endometrium, head and neck, kidney, liver, lung, muscle tissue, ovary, pancreas, prostate, skin, spleen, stomach, testicle, thyroid or uterus. In some embodiments, the cancer is a solid tumor. Examples of cancers that are solid tumors include, but are not limited to, pancreatic, bladder, non-small cell lung cancer (NSCLC), breast and ovarian cancers. In some embodiments, the cancer is pancreatic cancer, prostate cancer, or melanoma.

i. Radionuclide Coupled Elastin-Like Polypeptide

The method comprises administration of a composition comprising a first collection of self-assembling conjugates may include a first elastin-like polypeptide. As described above, elastin-like polypeptides (ELP) are thermally responsive polypeptides. “ELP” refers to a polypeptide comprising the pentapeptide repeat sequence (VPGX¹G)ₚ (SEQ ID NO: 3), wherein X¹ is any amino acid and n is an integer greater than or equal to 1.

The first elastin-like polypeptide may comprise an amino acid sequence of (VPGX¹G)ₚ (SEQ ID NO: 3), wherein X¹ is any amino acid, p is 1 to 500 and wherein the elastin-like polypeptide has a transition temperature below about 42° C. In some embodiments, p is an integer from 1 to 500, from 1 to 400, from 1 to 300, from 1 to 200, from 1 to 100, from 1 to 50, from 50 to 500, from 50 to 400, from 50 to 300, from 50 to 200, from 50 to 100, from 100 to 500, from 100 to 400, from 100 to 300, from 100 to 200, from 200 to 500, from 200 to 400, from 200 to 300, from 200 to 500, from 300 to 500, from 300 to 400, or from 400 to 500.

The first elastin-like polypeptide may comprise an amino acid sequence of (VPGX²G)_{q}(GᵣY)ₛ (SEQ ID NO: 4), wherein X² is any amino acid, q is 1 to 500, r is 0 to 10, and s is 1-250 and wherein the elastin-like polypeptide has a transition temperature below about 42° C. In some embodiments, q is an integer from 1 to 500, from 1 to 400, from 1 to 300, from 1 to 200, from 1 to 100, from 1 to 50, from 50 to 500, from 50 to 400, from 50 to 300, from 50 to 200, from 50 to 100, from 100 to 500, from 100 to 400, from 100 to 300, from 100 to 200, from 200 to 500, from 200 to 400, from 200 to 300, from 200 to 500, from 300 to 500, from 300 to 400, or from 400 to 500. In some embodiments, r is an integer from 0 to 10, from 0 to 9, from 0 to 8, from 0 to 7, from 0 to 6, from 0 to 5, from 0 to 4, from 0 to 3, from 0 to 2, from 0 to 1, from 1 to 10, from 1 to 9, from 1 to 8, from 1 to 7, from 1 to 6, from 1 to 5, from 1 to 3, from 1 to 2, from 2 to 10, from 2 to 9, from 2 to 8, from 2 to 7, from 2 to 6, from 2 to 5, from 2 to 4, from 2 to 3, from 3 to 10, from 3 to 9, from 3 to 8, from 3 to 7, from 3 to 6, from 3 to 5, from 3 to 4, from 4 to 10, from 4 to 9, from 4 to 8, from 4 to 7, from 4 to 6, from 4 to 5, from 5 to 10, from 5 to 9, from 5 to 8, from 5 to 7, from 5 to 6, from 6 to 10, from 6 to 9, from 6 to 8, from 6 to 7, from 7 to 10, from 7 to 9, from 7 to 8, from 8 to 10, from 8 to 9, or from 9 to 10. In some embodiments, s is an integer from 1 to 250, from 1 to 200, from 1 to 150, from 1 to 100, from 1 to 50, from 50 to 250, from 50 to 200, from 50 to 150, from 50 to 100, from 100 to 250, from 100 to 200, from 100 to 150, from 150 to 250, from 150 to 200, or from 200 to 250.

The first elastin-like polypeptide conjugated to the radionuclide may have phase transition behavior. Phase transition may refer to the aggregation, which may occur sharply and in some instances reversibly at or above the transition temperature. The Tₜ can be adjusted by varying the amino acid sequence of the elastin-like polypeptide, by varying the length of the polypeptide, or a combination thereof. The transition temperature may be below about 45° C., about 40° C., about 35° C., about 30° C., about 25° C., about 20° C., or about 15° C. The transition temperature may be below about 42° C.

In certain embodiments, the first elastin-like polypeptide comprises an amino acid sequence of (VPGVG)ₘ(GY)ₙ (SEQ ID NO: 1), wherein m is 50-250 and n is 1 to 50. In some embodiments, m is an integer from 50 to 200, from 50 to 150, from 50 to 100, from 100 to 250, from 100 to 200, from 100 to 150, from 150 to 200, from 150 to 200 or from 200 to 250. In some embodiments, m is 120. In some embodiments, n is an integer from 1 to 50, from 10 to 50, from 20 to 50, from 30 to 50, from 40 to 50, from 1 to 10, from 1 to 20, from 1 to 30 or from 1 to 40. In some embodiments, n is 7. In exemplary embodiments, m is 120 and n is 7.

The collection of self-assembling conjugates may include varying amounts of elastin-like polypeptide chains. For example, the assembly of self-assembling conjugates may include about 10 to about 200 elastin-like polypeptide chains per collection, such as about 10 to about 100 or about 50 to about 200.

The radionuclide may be any of the radioisotopes know in the art capable of providing irradiation through beta-particles, alpha-particles, gamma rays or Auger electrons. The radionuclide may include but is not limited to not limited to ¹³¹Cesium, ¹³⁷Cesium, ⁶⁰Cobalt, ¹⁹²Iridium, ¹²⁵Iodine, ¹³¹Iodine, ¹⁰³Palladium, ¹⁰⁶Ruthenium, ²²³Radium, ²²⁶Radium, ⁹⁰Yttrium, ¹⁷⁷Lutetium, ¹³¹Indium, ¹⁸⁶Rhenium, ⁸⁹Strontium, ¹⁵³Samarium, ³²Phosphorous, ²²⁵Actinium, ²¹¹Astatine, ²¹³Bismuth, and ²¹²Lead. In some embodiments, the radionuclide is ¹³¹Iodine.

Each of the first elastin-like polypeptides may be coupled with at least one radionuclide. In some embodiments, each of the first elastin-like polypeptides may be coupled with at least one, at least two, at least three, at least four, at least five, at least six, at least seven, at least eight, at least nine, or at least ten radionuclides.

The first collection of self-assembling conjugates may individually self-assemble into a variety of shapes and sizes. In some embodiments, first collection of self-assembling conjugates may individually self-assemble into a micelle. The micelles may be may be rod-shaped or spherical, or the collection may include combinations of differently shaped nanoparticles. The elastin-like polypeptides conjugated to a radionuclide may be defined by a critical micelle temperature. This is the minimal temperature at which micelles will form. Below the critical micelle temperature, micelles will not form. The critical micelle temperature may be below about 40° C., about 35° C., about 30° C., about 25° C., about 20° C. or about 15° C. In some embodiments the critical micelle temperature is below about 23° C.

The micelles may have a varying average hydrodynamic radius. In some embodiments, the nanoparticles have an average hydrodynamic radius of about 10 nm to about 100 nm, such as about 25 nm to about 75 nm or about 40 nm to about 60 nm. In some embodiments, the nanoparticle may have an average hydrodynamic radius of greater than 10 nm, greater than 20 nm, greater than 30 nm, greater than 40 nm, or greater than 50 nm. In some embodiments, the nanoparticle may have an average hydrodynamic radius of less than 100 nm, less than 90 nm, less than 80 nm, less than 70 nm, less than 60 nm, or less than 50 nm.

The micelle may also be described by its average radius of gyration. For example, the nanoparticle may have an average radius of gyration of about 10 nm to about 100 nm, such as about 25 nm to about 75 nm or about 40 nm to about 60 nm. In some embodiments, the nanoparticle may have an average radius of gyration of greater than 10 nm, greater than 20 nm, greater than 30 nm, greater than 40 nm, or greater than 50 nm. In some embodiments, the nanoparticle may have an average radius of gyration of less than 100 nm, less than 90 nm, less than 80 nm, less than 70 nm, less than 60 nm, or less than 50 nm.

The first elastin-like polypeptide conjugated to the radionuclide and the micelles they form may have phase transition behavior, wherein the micelles coacervate at a transition temperature or the micelle-coacervation transition temperature (Tₜ). Phase transition may refer to the aggregation, which may occur sharply and in some instances reversibly at or above the micelle-coacervation transition temperature. The Tₜ can be adjusted by varying the amino acid sequence of the elastin-like polypeptide, by varying the length of the polypeptide, or a combination thereof.

The micelle-coacervation transition temperature may be below about 60° C., about 55° C., about 50° C., about 45° C., about 40° C., about 35° C., about 30° C., about 25° C., about 20° C., or about 15° C. In some embodiments the micelle-coacervation transition temperature is below about 42° C.

Phase transition behavior may also enable purification of the conjugate using inverse transition cycling, thereby eliminating the need for chromatography. “Inverse transition cycling” refers to a protein purification method for polypeptides having phase transition behavior, and the method may involve the use of the conjugate's reversible phase transition behavior to cycle the solution through soluble and insoluble phases, thereby removing contaminants and eliminating the need for chromatography.

ii. Chemotherapeutic

The method comprises administration of a chemotherapeutic. The chemotherapeutic may be chosen from alkylating agents, anthracyclines, cytoskeletal disruptors or taxanes, epothilones, histone deacetylase inhibitors, topoisomerase inhibitors, kinase inhibitors, nucleotide analogs and precursor analogs, peptide antibiotics, platinum-based agents, retinoids, vinca alkaloids and derivatives, or combinations thereof. In some embodiments, the chemotherapeutic is a cytoskeletal disruptor or taxane. In exemplary embodiments, the chemotherapeutic is paclitaxel.

The chemotherapeutic may be contained in a second collection of self-assembling conjugates, wherein the second collection of self-assembling conjugates comprises the chemotherapeutic coupled to a second elastin-like polypeptide. The second elastin-like polypeptide may comprise an amino acid sequence SKGPG(X³GVPG)ₓWPC(GGC)_{z} (SEQ ID NO:2), wherein X³ is an amino acid or a combination of amino acids, x is 40 to 400 and z is 1 to 50. In some embodiments, the second elastin-like polypeptide comprises an amino acid sequence of SKGPG(X³GVPG)ₓWPC(GGC)_{z} (SEQ ID NO:2), wherein X³ is V:G:A in a ratio of 1:7:8. In some embodiments, x is an integer from 40 to 400, from 40 to 300, from 40 to 200, from 40 to 100, from 100 to 200, from 100 to 150, from 100 to 200, from 100 to 300 from 100 to 400, from 200 to 400, from 200 to 300, or from 300 to 400. In some embodiments, x is 160. In some embodiments, z is an integer from 1 to 50, from 10 to 50, from 20 to 50, from 30 to 50, from 40 to 50, from 1 to 10, from 1 to 20, from 1 to 30 or from 1 to 40. In some embodiments, z is 7. In certain embodiments, x is 160 and z is 7

The second collection of self-assembling conjugates may individually self-assemble into a variety of shapes and sizes. In some embodiments, the assembly of self-assembling conjugates may be a nanoparticle. The nanoparticle may be rod-shaped or spherical, or the collection may include combinations of differently shaped nanoparticles. In some embodiments, the nanoparticle is a micelle.

The second collection of self-assembling conjugates may include varying amounts of self-assembling polypeptide chains. For example, the assembly of self-assembling conjugates may include about 10 to about 200 self-assembling conjugates per assembly, such as about 10 to about 100, about 50 to about 200.

The second elastin-like polypeptide may also have phase transition behavior. The transition temperature (Tₜ) can be adjusted by varying the amino acid sequence of the polypeptide, by varying the length of the polypeptide, or a combination thereof. Phase transition may refer to the aggregation, which may occur sharply and in some instances reversibly at or above the transition temperature. The transition temperature may be below about 60° C., about 55° C., about 50° C., about 45° C., about 40° C., about 35° C., about 30° C., about 25° C., about 20° C., or about 15° C.

Phase transition behavior may also enable purification of the conjugate using inverse transition cycling, thereby eliminating the need for chromatography. “Inverse transition cycling” refers to a protein purification method for polypeptides having phase transition behavior, and the method may involve the use of the conjugate's reversible phase transition behavior to cycle the solution through soluble and insoluble phases, thereby removing contaminants and eliminating the need for chromatography.

### 4. Administration and Dosing

The disclosed compositions may be incorporated into pharmaceutical compositions suitable for administration to a subject (such as a patient, which may be a human or non-human) well known to those skilled in the pharmaceutical art. The pharmaceutical composition may be prepared for administration to a subject. Such pharmaceutical compositions can be administered in dosages and by techniques well known to those skilled in the medical arts taking into consideration such factors as the age, sex, weight, and condition of the particular subject, and the route of administration.

The pharmaceutical compositions may include pharmaceutically acceptable carriers. The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as, but not limited to, lactose, glucose and sucrose; starches such as, but not limited to, corn starch and potato starch; cellulose and its derivatives such as, but not limited to, sodium carboxymethyl cellulose, ethyl cellulose and cellulose acetate; powdered tragacanth; malt; gelatin; talc; excipients such as, but not limited to, cocoa butter and suppository waxes; oils such as, but not limited to, peanut oil, cottonseed oil, safflower oil, sesame oil, olive oil, corn oil and soybean oil; glycols; such as propylene glycol; esters such as, but not limited to, ethyl oleate and ethyl laurate; agar; buffering agents such as, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants such as, but not limited to, sodium lauryl sulfate and magnesium stearate, as well as coloring agents, releasing agents, coating agents, sweetening, flavoring and perfuming agents, preservatives and antioxidants can also be present in the composition, according to the judgment of the formulator. The route by which the composition is administered and the form of the composition will dictate the type of carrier to be used.

The compositions disclosed herein can be administered prophylactically or therapeutically. In prophylactic administration, the composition can be administered in an amount sufficient to induce a response. In therapeutic applications, the composition is administered to a subject in need thereof in an amount sufficient to elicit a therapeutic effect. An amount adequate to accomplish this is defined as “therapeutically effective dose.” Amounts effective for this use will depend on, e.g., the particular composition of the conjugate regimen administered, the manner of administration, the stage and severity of the disease, the general state of health of the patient, and the judgment of the prescribing physician.

The compositions disclosed herein can be administered by methods well known in the art as described in Donnelly et al. (Ann. Rev. Immunol. 1997, 15, 617-648); Felgner et al. (U.S. Pat. No. 5,580,859, issued Dec. 3, 1996); Felgner (U.S. Pat. No. 5,703,055, issued Dec. 30, 1997); and Carson et al. (U.S. Pat. No. 5,679,647, issued Oct. 21, 1997). One skilled in the art would know that the choice of a pharmaceutically acceptable carrier, including a physiologically acceptable compound, depends, for example, on the route of administration.

The compositions disclosed herein may conveniently be presented in a single dose or as divided doses administered at appropriate intervals, for example, as two, three, four or more sub-doses per day. The sub-dose itself may be further divided, e.g., into a number of discrete loosely spaced administrations.

As will be readily apparent to one skilled in the art, the useful in vivo dosage to be administered and the particular mode of administration will vary depending upon the age, weight, the severity of the affliction, and mammalian species treated, the particular compounds employed, and the specific use for which these compounds are employed. The determination of effective dosage levels, that is the dosage levels necessary to achieve the desired result, can be accomplished by one skilled in the art using routine methods, for example, human clinical trials, in vivo studies and in vitro studies.

Dosage amount(s) and interval(s) may be adjusted individually to provide plasma levels of the molecule which are sufficient to maintain the modulating effects, or minimal effective concentration (MEC). The MEC will vary for each molecule but can be estimated from in vivo and/or in vitro data. Dosages necessary to achieve the MEC will depend on individual characteristics and route of administration. However, assays well known to those in the art can be used to determine plasma concentrations. Dosage intervals can also be determined using MEC value. Compositions should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the symptoms to be treated and the route of administration. Further, the dose, and perhaps dose frequency, will also vary according to the age, body weight, and response of the individual patient. A program comparable to that discussed above may be used in veterinary medicine.

A therapeutically effective amount of the compositions may be administered alone or in combination with a therapeutically effective amount of at least one additional therapeutic agents. In some embodiments, effective combination therapy is achieved with a single composition or pharmacological formulation that includes both agents, or with two distinct compositions or formulations, administered at the same time, wherein one composition includes a compound of this invention, and the other includes the second agent(s). Alternatively, in other embodiments, the therapy precedes or follows the other agent treatment by intervals ranging from minutes to months.

A wide range of second therapies may be used in conjunction with the compounds of the present disclosure. The second therapy may be a combination of a second therapeutic agent or may be a second therapy not connected to administration of another agent. Such second therapies include, but are not limited to, surgery, immunotherapy, radiotherapy, or a second chemotherapeutic agent.

### 5. Examples

It will be readily apparent to those skilled in the art that other suitable modifications and adaptations of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

### Example 1: Materials and Methods

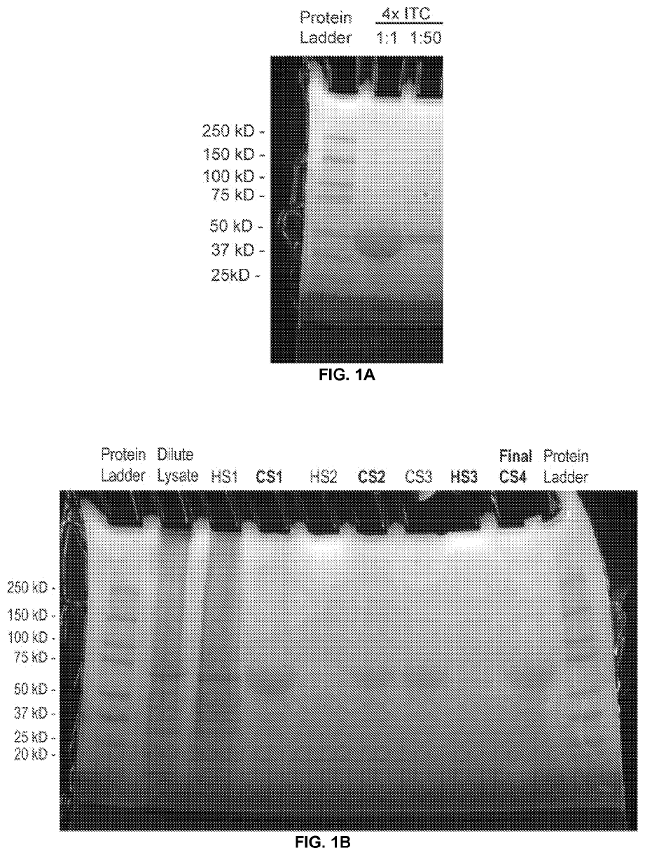

Synthesis and purification of ELP. Depot-forming ELP was recombinantly synthesized by encoding the DNA sequence for the repetitive polypeptide (VPGVG)₁₂₀(GY)₇ (SEQ ID NO:1) in a pET-24a+ vector (Novagen Inc.) and was expressed in a competent BL21(DE3) strain of E. coli (Edge BioSystems), as previously described. The theoretical molecular weight (MW) of the construct was 50,682 Da. Briefly, E. coli were cultured in Terrific Broth (VWR Life Science), supplemented with 4 mL/L glycerol and 45 μg/mL kanamycin. Overexpression of the ELP was induced after 8 h of culture by addition of 0.1 mM IPTG (GoldBio, Inc.). After 24 h, the E. coli was collected, lysed by sonication, and the ELP isolated from the soluble fraction of the cell lysate using 4 rounds of inverse transition cycling (ITC) purification. Each cycle of ITC consisted of the addition of 1 mM NaCl to the soluble fraction of the cell lysate, followed by centrifugation at 14,000 rpm and 35° C. to pellet the aggregated ELP. The isolated pellet was then dissolved in cold PBS and centrifuged at 4° C. and 14000 rpm to remove insoluble contaminants. After 4 rounds of ITC, the purified ELP was dialyzed into H₂O. Endotoxins were removed by incubating the ELP in Detoxi-gel resin (Thermo Fisher Scientific) packed in PD-10 columns (Thermo Fisher Scientific). Final endotoxin content was verified to be <0.25 EU/mL, in accordance with USP-NF standards, using the LAL gel clot assay (Lonza). ELP purity was verified to be greater than 95% using 4-20% HCl-Tris protein gels (BioRad) stained with 0.5M CuCl2 after SDS-PAGE (FIG. 1).

The micelle-forming ELP used for delivery of paclitaxel was comprised of a chimeric polypeptide (CP) with the amino acid sequence SKGPG(XGVPG)₁₆₀WPC(GGC)₇ (SEQ ID NO:2) with the guest residue X V:G:A in a ratio of 1:7:8. The MW of the construct was 61,663 Da. The expression of the CP followed the same procedure as the depot-forming ELP with two notable exceptions: the temperature for the hot centrifugation steps was carried out at 75° C. and ELP was re-solubilized in cold PBS with 50 mM tris(2-carboxyethyl)phosphine hydrochloride (TCEP, Sigma Aldrich) to reduce disulfide bond formation. Once purification was complete, both ELP constructs were lyophilized in endotoxin free water and stored at −80° C.

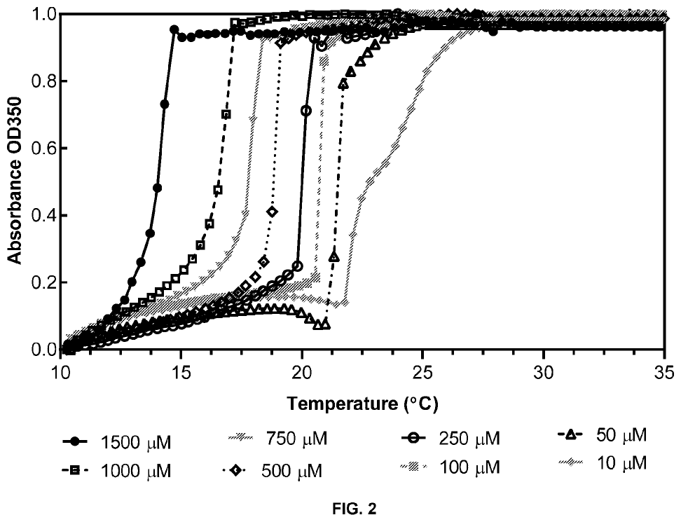

Characterization of ELP properties. The thermal properties of each ELP were characterized by measuring the optical turbidity at 350 nm as a function of temperature on a multi-well Cary 300 UV-vis spectrophotometer (Varian Instruments, Palo Alta, Calif.). The temperature was raised from 15° C. to 95° C. at a rate of 1° C./min, with the turbidity measured every 0.3° C. (FIG. 2). The transition temperature (Tₜ) was defined as the temperature at which the first derivative of absorbance with respect to temperature reached a maximum.

The hydrodynamic radius (Rh) was measured by dynamic light scattering (DLS) on a Protein Solutions DynaPro DLS System (Wyatt Technology). CP-PTX samples were formulated at 50 μM and then sterile filtered using 0.2 μm filters to remove dust particles. DLS scans were acquired for 15 s and the light scattering data was fit using a regularization fit of the autocorrelation function. Static light scattering (SLS) was performed on an ALV/CGS-3 Compact Goniometer system (ALV GMBH, Langen) at 5° angle increments between 30° and 150°. Three different 15 s scans were performed and averaged with a dRate %<5%. The partial Zimm plot was analyzed to determine the radius of gyration (RG) and apparent MW of the CP-PTX.

¹³¹Iodine radionuclide conjugation to depot-forming ELP. Conjugation of ¹³¹Iodine was carried out using the direct Iodogen oxidation method. Briefly, Na¹³¹I was purchased from Perkin Elmer and reacted with 500 μM ELP in Pierce® IODOGEN pre-coated tubes (Thermo Fisher Scientific) on ice for 30 min. Unreacted iodine was removed from the mixture using Zeba Spin Desalting Columns, 40K MWCO (Thermo Fisher Scientific). Next, the solution was centrifuged at 1000 rcf for 4 min at 4° C. Radioactivity levels were verified using the AtomLab 400 dose calibrator (Biodex). The ¹³¹I-ELP conjugate was then mixed with unreacted ELP to bring the final ELP concentration to 1000 μM and the radioactivity to its desired level.

Conjugation of chimeric polypeptide micelles to paclitaxel (CP-PTX). Paclitaxel (PTX) was conjugated to the CP by a multi-step reaction utilizing levulinic acid (LEV) and N-ε-maleimidocaproic acid hydrazide (EMCH) as previously described. Briefly, 38.74 mg of LEV (Tokyo Chemical Industries) were reacted with 75 mg of N,N′-dicyclohexylcarbodiimide (Sigma Aldrich) in anhydrous DMF for 30 min at −20° C. 200 mg of paclitaxel (Ark Pharm) was then solubilized in 500 μL anhydrous DMF by vortexing. The PTX was then transferred to the LEV reaction mixture, amounting to a molar reaction ratio of 2:1 LEV to PTX. 10 mg of 4-dimethylaminopyridine (Alfa Aesar) was then added, the mixture protected from light, and then left to react for 12 h at 4° C. The reaction products were then filtered and the DMF was completely evaporated. The PTX-LEV conjugate was then purified by column chromatography (Silica Gel 60, Alfa Aesar) using a 0.5%-1.5% methanol in chloroform eluent gradient. Purity was assessed by thin layer chromatography. Final products were dried using a rotovap and stored at −20° C., protected from light.

Next, the PTX-LEV conjugate was dissolved in anhydrous methanol in a round bottom flask. For every 25 mg of PTX-LEV, 10.9 mg of EMCH (Thermo Fisher Scientific) was added to the solution. Additional methanol was added as necessary to ensure full solubility of the contents. The mixture was covered in aluminum foil and transferred to a 48° C. oil bath. The mixture was left to react for 48 h. After allowing the reaction to return to room temperature, the mixture was then purified using column chromatography (Silica Gel 60) with a 0.8%-1.8% methanol in chloroform eluent gradient. The eluent was collected in fractions and composition assessed by thin layer chromatography. The final PTX-LEV-EMCH product was dried, weighed, and then resuspended in DMF.

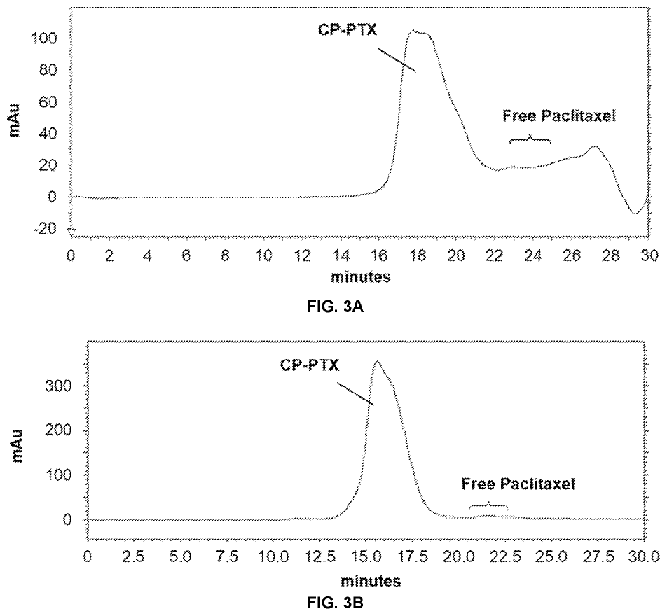

Finally, lyophilized CP was dissolved in 50 mM of NaPO₄ and 100 mM of TCEP in a round bottom flask. 180 mg of ELP was used for every 50 mg of PTX-LEV-EMCH in the conjugation reaction. DMF was added to the ELP at a 1:1 volume mixture, and then the PTX-LEV-EMCH was transferred to the mixture and allowed to react for 12 h at room temperature. After 12 h, unreacted PTX-LEV-EMCH was separated from the product by centrifugation at 14000 rpm for 10 min at 10° C. The supernatant was further purified by dilution into 30% acetonitrile in PBS and repeated centrifugal ultrafiltration with an Amicon Ultra-15 filter unit (MWCO 10 kDa) to remove unconjugated PTX. Purity was checked by analytical HPLC with an OHPak KB-804 size exclusion column (Shodex). Purification was deemed complete once the residual unbound paclitaxel content was <5% of the purified product, as determined by integrating the area under the curve of the HPLC trace (FIG. 3). Purified CP-PTX (defined as ≥95% pure after ultrafiltration) was washed with NH₄HCO₃ in two final ultrafiltration steps, lyophilized, and stored at −80° C.

Cell lines and Animal models. The BxPc3-luc2 human pancreatic tumor line was purchased from Perkin Elmer. MIA PaCa-2 and AsPc-1 were obtained from the Duke Cell Culture Facility, a repository of ATCC cell lines that is available to Duke University researchers. All cell lines were verified to be murine pathogen free through IMPACT III testing (IDEXX BioResearch). BxPc3-luc2 and AsPc-1 cells were cultured using RPMI 1640 media supplemented with 10% HI-FBS. MIA PaCa-2 cells were passaged using Dulbeco's Modified Eagle Medium (DMEM) supplemented with 5% horse serum and 10% FBS. To passage cells, 0.25% trypsin/EDTA (Thermo Fisher Scientific) was used to detach cells from the culture flasks upon reaching 80-85% confluency.

Male athymic, nu/nu mice were purchased from the Duke University Immunoincompetent Rodent and Biohazard Facility. Mice were obtained at 6-8 weeks old in age and were housed in the Duke Cancer Center Isolation Facility. Animals were subjected to standard 12 h/12 h light/dark cycles in a BSL2 barrier facility with sterile food and water provided ad libitum.

Two different types of tumor models were used in various studies. Subcutaneous tumor models were established by first preparing cells in an 800/c v/v Matrigel (Corning), 20% DMEM solution of 1×10⁶ cells per 15 μL. 2×10⁶ cells were then injected into the subcutaneous flank on the right hind leg of the mouse. Tumor growth was monitored using digital hand calipers, where Volume=L*W²*π/6. For orthotopic pancreatic tumor models, only the bioluminescent BxPc3-luc2 cell line was utilized. Cells were similarly prepared in an 80% v/v Matrigel: 20% DMEM solution at 1×106 cells per 10 μL. Survival surgery was performed to access the pancreas and inoculation of tumor cells was performed using the cinched suture technique, as previously published. Briefly, a 5-0 bioabsorbable suture was used to cinch a portion of the pancreatic tissue, allowing the cells to be injected into a single site without dissemination throughout the organ. Mice were maintained under continuous isoflurane anesthesia throughout the surgical procedure using 2.5% isoflurane in a 2 L/min 02 feed. Upon completion of surgery, mice were provided 0.05 mg/kg buprenorphine for pain management, injected intraperitoneally with 700 μL of PBS for dehydration prophylaxis, and antibiotic ointment was administered on the surgical closure site.

In vitro cytotoxicity assessment. All cell lines were cultured in monolayers in sterile, vented culture flasks (Corning). Cells were passaged, collected, and then counted with 0.4% Trypan Blue (Thermo Fisher Scientific). Cells were then formulated in their respective media and plated in 96-well plates at 5,000 cells per well in 100 μL. After allowing cells to incubate at 37° C. for 12 h, drugs were then added to the wells. Each drug was formulated at the highest concentration in the cell media and then serially diluted. A PTX equivalent concentration between 10-4 and 10-12 M of each construct was plated in quadruplicate and allowed to incubate for 72 h. 20 μL of CellTiter 96® AQueoous One solution (Promega) was then added to each well. After 1.5 h, the absorbance of each plate was read at 490 nm and normalized to untreated cells to determine the relative cell survival. The half effective concentration (EC50) was calculated as the inflection between the curve's maxima and minima. The IC50, or half maximal inhibitory concentration, was measured at the point where cell survival was equal to 50% of the cell survival.

Tumor regression models. The differential impact of ¹³¹I-ELP and CP-PTX on combination therapy was determined through two dose-escalation studies. In the first radioactivity dose-escalation study, 40 mice were inoculated with subcutaneous BxPc3-luc2 tumors, grown to 125 mm³, and divided into 8 groups (n=5). All mice receiving CP-PTX were all given a single i.v. dose at half MTD (25 mg/kg) at the same time as the tumors received ¹³¹I-ELP brachytherapy treatment. ¹³¹I-ELP was intratumorally injected at one-third the tumor volume at doses of 3.3, 6.6, and 10.0 μCi/mm³. Groups consisted of untreated tumors, CP-PTX only treatment, brachytherapy only at the three doses, and combination therapy at the three ¹³¹I-ELP doses. In the CP-PTX dose escalation study, 50 mice were similarly inoculated with subcutaneous BxPc3-luc2 tumors and grown to 125 mm³. Mice were randomized and divided into treatment groups (n=5) consisting of untreated tumors, brachytherapy only, CP-PTX only at different doses, and combination therapy at different CP-PTX doses. The ¹³¹I-ELP brachytherapy dose remained constant in this experiment at 3.3 μci/mm³. CP-PTX dose groups consisted of single i.v. bolus infusions at 12.5 mg/kg, 25 mg/kg, and 50 mg/kg. A separate CP-PTX dose group consisted of administering two 25 mg/kg injections of CP-PTX, administered one week apart. Treatment response was assessed by tracking tumor volume over time, categorizing the response according to RECIST criteria, body weight change, and overall animal survival.

Next, the various elements of the combination therapy strategy were systematically replaced with the equivalent clinical standard of care for comparison. First, 18 mice were subcutaneously inoculated with BxPc3-luc2 tumors to evaluate ¹³¹I-ELP when combined with Abraxane. Tumors were grown to ˜125 mm³ in size and mice were randomized into three groups (n=6) of untreated tumors, an Abraxane only control, and ¹³¹I-ELP administered with Abraxane. ¹³¹I-ELP was delivered at the optimum dose of 10.0 μci/mm³ and one-third tumor volume. Abraxane was intravenously injected at 12.5 mg/kg of PTX equivalent, once weekly for 4 weeks. In a separate study, 28 mice were inoculated with subcutaneous BxPc-luc2 tumors to evaluate the synergistic response of clinical EBRT when combined with CP-PTX. Mice were divided into four groups (n=7) including untreated controls, CP-PTX only treatment, EBRT only treatment, and EBRT combined with CP-PTX. The EBRT dose fractionation was selected to match current hypofractionated regimens used clinically for treatment of pancreatic cancer: five fractions of 5 Gy were administered every 2-3 days. CP-PTX was administered i.v. at 12.5 mg/kg once weekly for four weeks. Consistent response criteria and end-points for both studies were maintained in accordance with previous regression studies. To evaluate whether observed effects were synergistic, the Bliss Independence framework was applied as described in the Example 9. Synergy was determined to be significant for p<0.05.

Final regression studies were then performed using the optimized synergistic combination therapy to assess whether effects remained consistent across diverse genetic and phenotypic models of pancreatic cancer. The doses consisted of ¹³¹I-ELP at 10.0 μCi/mm³ and 12.5 mg/kg of CP-PTX given q.w. for 4 weeks.

12 mice were subcutaneously inoculated on the hind flank with MIA PaCa-2 cells and allowed to grow to a volume of 125 mm³. Mice were then randomized and divided into 2 treatment groups: untreated and optimized combination therapy (n=6). For AsPc-1 tumors, 14 mice similarly received hind flank subcutaneous inoculations, were grown to tumor volumes of 125 mm³, and then were divided into 2 groups (n=7): untreated and optimized combination therapy. For the orthotopic BxPc3-luc2 model, 36 mice were surgically inoculated with 1×106 tumor cells directly in the pancreas. Tumors were grown for 21 days and then mice were divided then into 6 groups (n=6): untreated controls, i.v. CP-PTX at 12.5 mg/kg q.w., CP-PTX at 25 mg/kg q.w., ¹³¹I-ELP only, and combination therapy at both CP-PTX doses. Tumor response was tracked by monitoring bioluminescence by i.p. injection of potassium luciferin (GoldBio, Inc) and measuring the maximal flux using the IVIS Lumina XR (Xenogen). Mice were sacrificed when tumor signal exceeded 1×10¹⁰ photons/sec; corresponding to a tumor volume greater than 1750 mm³. Blood samples were collected during this study to assay for circulating α-amylase levels, an indicator of pancreatic inflammation and damage. Healthy mice (n=5) and mice with tumors but were not treated (n=5) served as 0 d control comparisons. All treatment groups had blood samples collected at time points corresponding to 30 min, 10, 20, 30, and 40 days after treatment. Levels of α-amylase were quantified using the colorimetric Amylase Activity Assay Kit (Sigma Aldrich) by measuring absorbance at 405 nm.

External beam X-ray irradiation procedures. External beam radiation was delivered to all animals using the 225 kVp X-RAD CX225 micro-CT/micro-irradiator within Duke's GSRBH animal facility. Mice were first anesthetized using a continuous feed of 2.5% isoflurane in 2 L/min 02. X-ray imaging was first for image-guided irradiation to the subcutaneous hind leg tumor. 5 Gy of radiation was then delivered over 50 s using anterior and posterior opposed photon beams at 225 kVp and 13 mA. Mice received one fraction every other day until a total dose of 25 Gy had been delivered. For mice in groups receiving concurrent CP-PTX chemotherapy, CP-PTX was injected 30 min prior to irradiation.

Fluorescent nanoparticle accumulation studies. Tumor-specific uptake of nanoparticles was enabled by fluorescent labeling of CP-PTX at the N-terminal amine on the micelle corona. SulfoCy5.5 NHS ester (Lumiprobe) was first dissolved in 500 μL of DMSO in 4.5 mL of PBS. The fluorophore was then reacted at an 8:1 molar ratio of CP-PTX for 12 h at 4° C. The reaction mixture was purified using 10 kDa MWCO Amicon-Ultracentrifugation filter units (Millipore). Final formulation concentration was evaluated by measuring the relative absorbance at 280 nm (ELP, ε=5690) and 675 nm (sulfoCy5.5, ε=195000). SulfoCy5.5 concentration was determined to be 113.6 μM yielding a labeling ratio of ˜1.5 sulfoCy5.5 per CP-PTX nanoparticle.

BxPc3-luc2 tumors were grown subcutaneously on the hind flank of 15 athymic nu/nu mice. Upon reaching a target size of 100 mm³, mice were randomized into three groups (n=5) and received either ¹³¹I-ELP treatment at 10 μCi/mm³, hypofractionated EBRT therapy, or remained untreated. SulfoCy5.5-CP-PTX was injected i.v. at ˜25 mg/kg on the initial day of treatment with a subsequent dose 7 days later. Fluorescent flux was tracked at the tumor site over time using the IVIS Lumina XR (Perkin Elmer) and normalized to individual tumor sizes as measured with calipers.

Biodistribution study. Orthotopic BxPc3-luc2 tumors were implanted into 20 athymic nu/nu mice and divided into five groups (n=4). Tumors were grown for 21 days to reach a target size of 150 mms ¹³¹I-ELP was then injected intratumorally at a radioactivity dose of 10 μCi/mm³. A set of ¹³¹I-ELP samples were aliquoted in triplicate; creating an activity standard curve ranging from 6×10-8 to 1.8×10-5 Ci. Mice were then euthanized at 30 min, 24 h, 48 h, 72 h, and 196 h. Tissues were dissected from each mouse, weighed, and stored at −20° C. for analysis. These tissues included blood, skin, muscle, heart, lungs, liver, kidneys, stomach, small intestines, and the large intestines. The ¹³¹I-ELP activity was counted using a Wallac Wizard 3 automatic gamma counter (Perkin Elmer). The results were normalized to the mass of each tissue sample and decay-corrected to determine exposure activities. Cumulative exposure was determined using the dose formula described in Example 11.

Histochemical pathology of treated tumor. Orthotopic BxPc3-luc2 tumors were inoculated in 12 athymic nude mice, and another 11 mice were subcutaneously inoculated with BxPc3-luc2 tumors. Tumors were grown for 10 days to reach the desired size of 100-125 mm³. At that time, mice were randomized into groups and received the following treatments. Mice with orthotopic tumors either remained untreated (n=2), received i.v. injections of CP-PTX (n=2), were only treated with ¹³¹I-ELP depots (n=3), or received the combination therapy of 11-ELP and CP-PTX (n=4). Mice with subcutaneous tumors either received no treatment (n=2), only received EBRT (n=4), or received the combined therapy of EBRT with CP-PTX (n=5). CP-PTX was administered intravenously once weekly at 12.5 mg/kg paclitaxel equivalent to its respective groups. EBRT was given as five, 5 Gy fractions every other day while ¹³¹I-ELP was injected intratumorally at 10 μCi/mm³. All mice were euthanized 12 days after initiating treatment, and tumors were removed and preserved in formalin. Pancreatic tissue from healthy mice (n=2) was also collected as a histological reference. All tissue samples were stored for 6 months to allow for complete ¹³¹Iodine decay prior to histology.

Once safe for handling, tissue samples were paraffin embedded, prepared in 7 μm sections, and stained for histological examination. IHC markers included hematoxylin and eosin staining, Masson Trichrome, CD-31 (ThermoFisher, #PA5-16301), CD-144 (ThermoFisher, #36-1900), anti-Claudin-4 (ThermoFisher, #PA5-16875), and TUNEL (Millipore, #S7100). All samples were then imaged using the Zeiss Axio Imager 2 Upright Microscope (Zeiss) in the Duke Light Microscopy core facility. Stitched images of whole tumor sections were analyzed with ImageJ software to determine the relative area of expression for each stain. Briefly, images were spectrally deconvoluted according to the methyl green counter stain. A binary mask of the immunohistochemistry stain was then created, and the percent area calculated as normalized to the full tumor specimen area. Tumor histology and immunohistochemical stains were blindly reviewed by a board-certified anatomic pathologist (KS) at the Duke University Medical Center.

### Example 2: Effects of Paclitaxel and ¹³¹I-ELP on Pancreatic Tumor Regression

The human pancreatic cell line BxPc3 was selected as the initial pancreatic tumor model because it is one of the most frequently used cell lines in the study of pancreatic cancer and its response to radio-chemotherapy agents has been extensively documented. The cell line is typically responsive to in vitro testing with many cytotoxic anticancer agents. However, in vivo tumor xenografts of this cell line have proven highly resistant to these treatments; including EBRT, paclitaxel, gemcitabine, erlotinib, cetuximab, and oxaliplatin. This resistance, which mirrors clinical responses to treatment, has largely been attributed to the high stromal content, hypoxia, and hypovascularization of the tumor environment of the BxPc3 tumors.

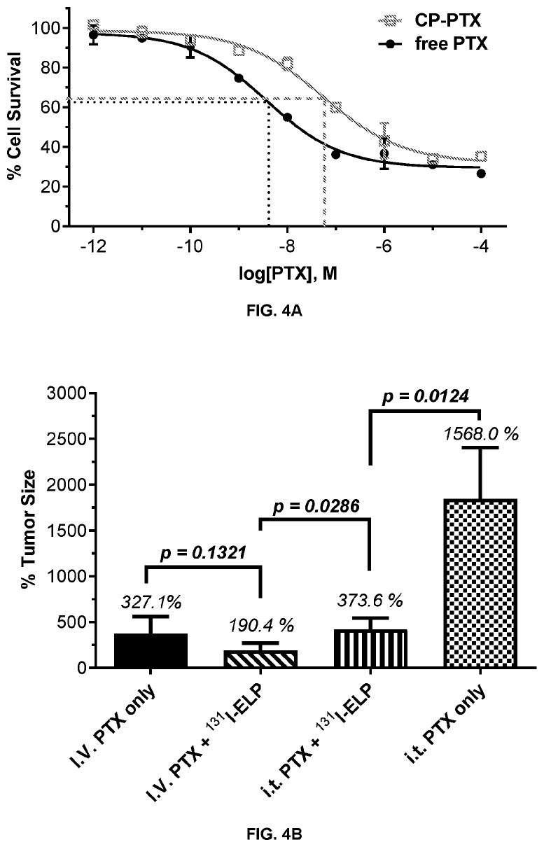

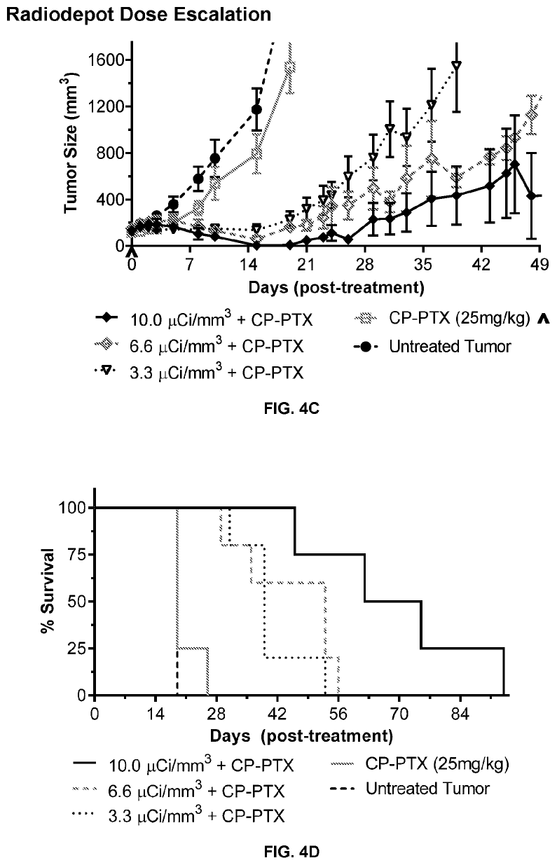

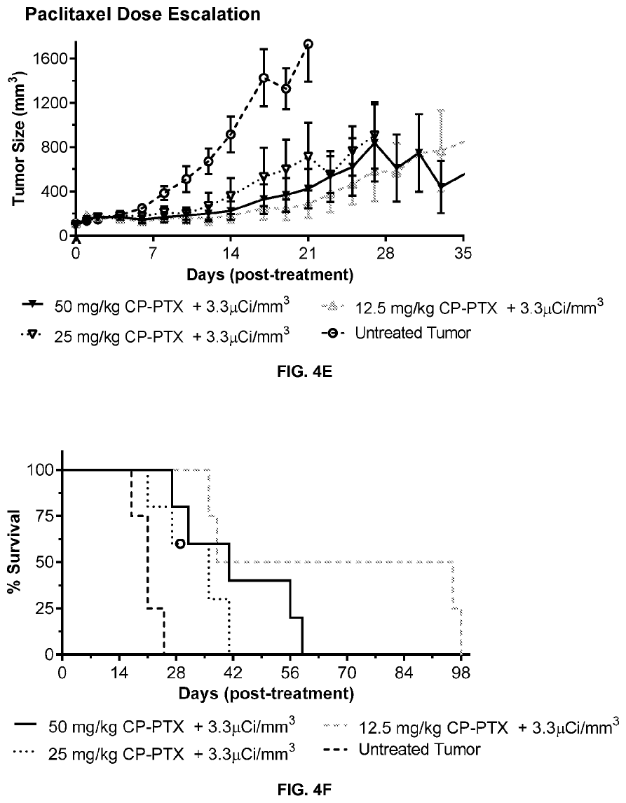

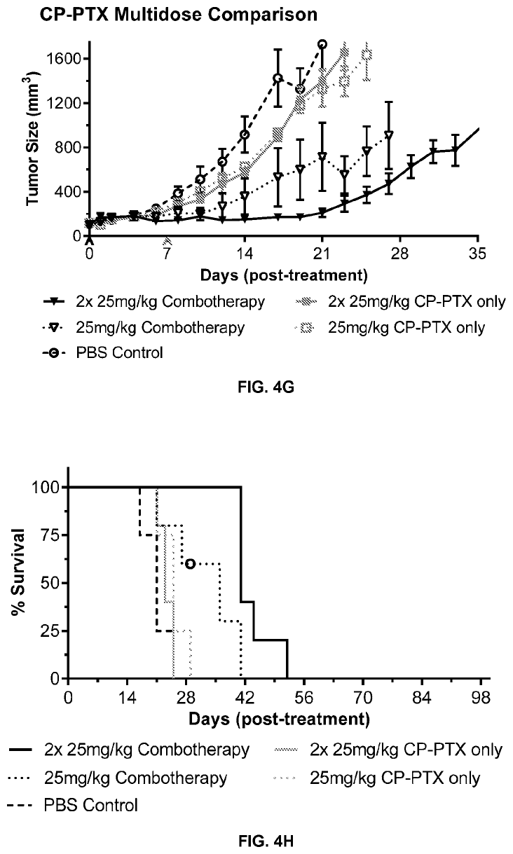

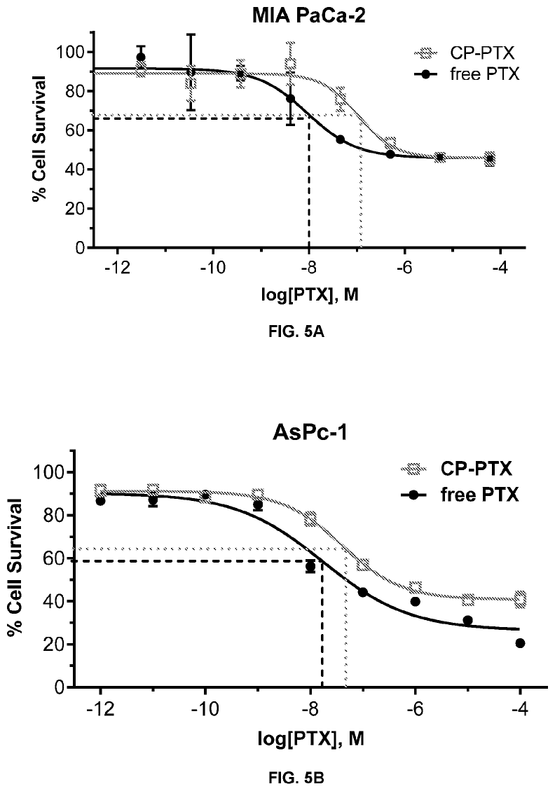

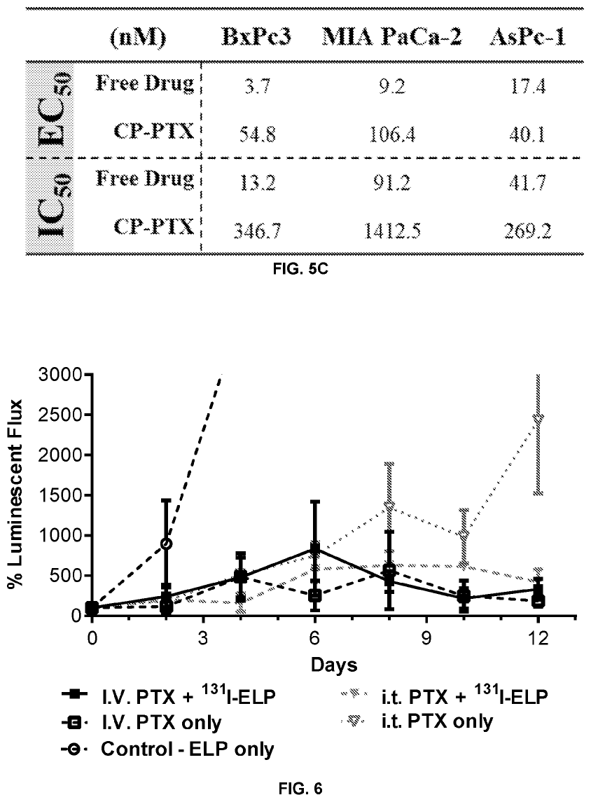

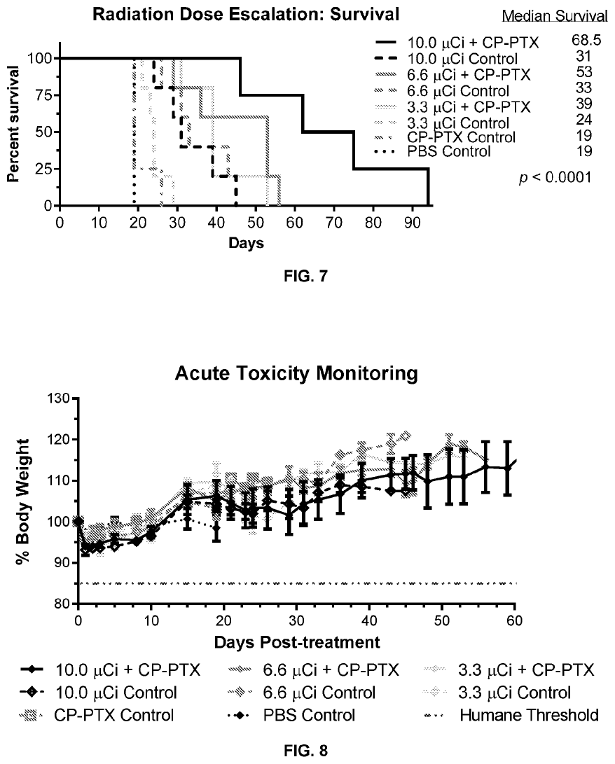

The effect of paclitaxel on BxPc3 cytotoxicity was validated with an MTS cell proliferation assay (FIG. 4A). Cells were incubated for 72 h with either free paclitaxel (PTX) or a micelle formulation, CP-PTX. CP-PTX was synthesized by covalently conjugating 2-3 molecules of PTX to a chimeric polypeptide through an acid-sensitive bond. PTX exhibited a relative half-inhibitory concentration (EC50) of 3.7 nM while that of CP-PTX was 54.8 nM. The reduced EC50 for CP-PTX was consistent with previous results. Moreover, CP-PTX was more cytotoxic than Abraxane in the same cell line. For both formulations, cytotoxicity appeared to reach a threshold at ˜30% cell survival. In addition to BxPc3 cells, MIA PaCa-2, and AsPc-1 were also selected as they are well characterized, highly resistant human pancreatic cell lines with different genetic profiles common across cancer patients. As shown in FIG. 5, paclitaxel exhibited nanomolar cytotoxicity in each cell line. Depending on the cell line, the CP-PTX micelle formulation exhibited 3- to 15-fold reduction in cytotoxicity.

Next, the optimal delivery route to combine paclitaxel with ¹³¹I-ELP in an in vivo setting was determined. Paclitaxel is typically delivered systemically through intravenous (i.v.) injection, but the compositions and methods disclosed herein also allow for simultaneous intratumoral (i.t.) infusion with ¹³¹I-ELP brachytherapy. In a pilot study, both treatment options in orthotopic BxPc3 tumors in athymic nu/nu mice were explored. Tumors were grown for 21 days to reach a size between 125-150 mm³ based on luminescent flux. Mice were randomized into 4 groups (n=5) and received either i.v. CP-PTX or i.t. CP-PTX treatment, with and without ¹³¹I-ELP brachytherapy. CP-PTX was administered as a single bolus at 25 mg/kg while ¹³¹I-ELP was injected at a radioactivity dose of 1.5 μCi/mm³. Mice were sacrificed after 12 d and tumor size was measured to assess treatment response.

FIG. 6 shows that luminescent tracking did not provide insightful conclusions about either treatment, as the noise associated with bioluminescence monitoring in deep tissue orthotopic tumor model obscured conclusions. Significant differences in tumor growth were observed (Table 1) between all treatment groups (p<0.001, one-way ANOVA). As seen in FIG. 4B, intratumoral delivery of CP-PTX was less effective than i.v. CP-PTX at controlling tumor growth, both as a single agent or when combined with ¹³¹I-ELP. Combination therapy produced superior tumor responses with both delivery methods, although i.v. CP-PTX inhibited tumor size significantly better than the co-injected, intratumoral formulation (p<0.05, Tukey's post-hoc t-test). Based on these results, the synergistic potential of ¹³¹I-ELP with systemic paclitaxel delivery was investigated next.

TABLE 1

| Treatment | Tumor | Body Wt | | ¹³¹I-ELP | CP-PTX | CP-PTX | Tumor - 12 d | % Initial |
|---|---|---|---|---|---|---|---|---|
| Group | Size | (g) | n | Dose | (mg/kg) | Frequency | (mg) | Tumor |
| IV. CP-PTX + ¹³¹I-ELP | 169.2 | 31.6 ± 2.4 | 4 | 1.30 μCi/mg | 25 mg/kg | 1× | 287.2 | 190.4% |
| I.V. CP-PTX only | 68.8 | 28.5 ± 1.5 | 3 | n/a | 25 mg/kg | 1× | 389.0 | 327.1% |
| i.t. CP-PTX + ¹³¹I-ELP | 183.8 | 30.7 ± 2.5 | 4 | 1.53 μCi/mg | 25 mg/kg | 1× | 516.8 | 373.6% |
| i.t. CP-PTX only | 65.9 | 30.3 ± 0.6 | 3 | n/a | 25 mg/kg | 1× | 1121.7 | 1568.0 |

The effects of ¹³¹I-ELP brachytherapy and i.v. CP-PTX were explored in a series of controlled in vivo dose escalation studies using athymic nu/nu mice (n=5). BxPc3-luc2 tumors were subcutaneously implanted in the hind flanks of nu/nu mice and allowed to grow to a size of 125 mm³. The effect of ¹³¹I-ELP brachytherapy dose on tumor regression was investigated; either alone or in combination with a constant dose of CP-PTX. Radioactivity doses of 3.3 μCi/mm³, 6.6 μCi/mm³, and 10.0 μCi/mm³ were used for the brachytherapy groups. ¹³¹I-ELP was intratumorally infused at 180 μL/min at a volume one-third the size of the target tumor. For combination groups, the CP-PTX administered as a single i.v. injection at an equivalent dose of 25 mg/kg of paclitaxel. Treatments were administered in rapid succession, so as not to introduce timing as a variable.

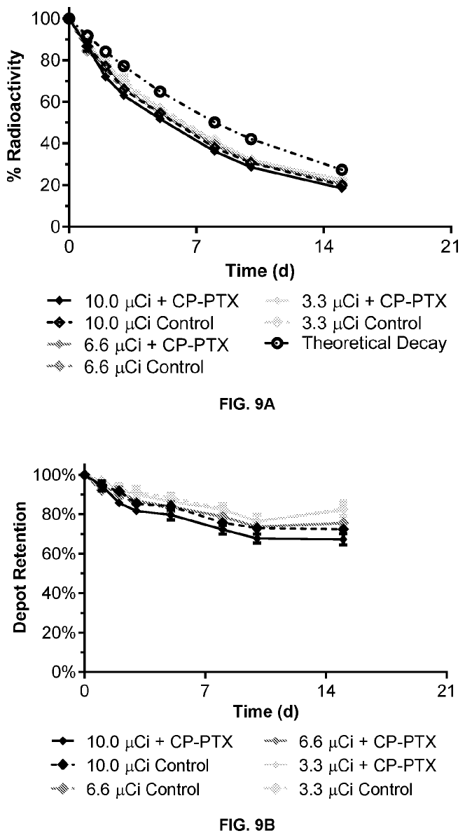

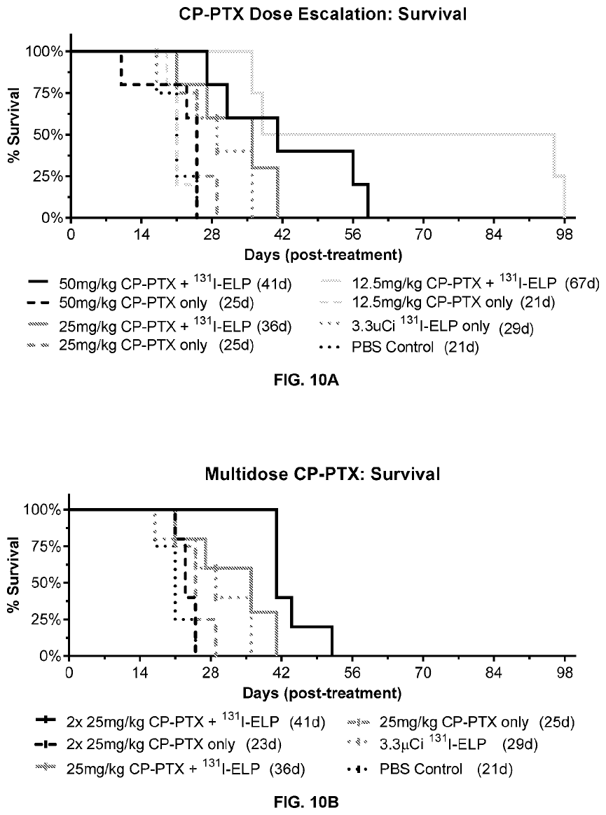

Tumors treated with monotherapies of CP-PTX (25 mg/kg) or ¹³¹I-ELP at 3.3 μCi/mm³ were indistinguishable from untreated tumors (Table 2). ¹³¹I-ELP monotherapy at higher doses of 6.6 and 10.0 μCi/mm³ did induce tumor growth inhibition, but the effects were modest. In contrast, the combination of ¹³¹I-ELP with CP-PTX (FIG. 4C), significant tumor regression was achieved compared to all monotherapy controls (p<0.0001, 2-way repeated-measure ANOVA with Tukey post-hoc analysis). The 3.3 μCi/mm³ combination therapy group achieved a 40% overall response rate (ORR, partial response+complete response) and a prolonged median survival of 39 d compared to 19 d for untreated tumors (FIG. 4D, p<0.05, Mantel-Cox log-rank test). The 6.6 μCi/mm³ combination therapy group achieved an 80% ORR with a median survival of 53 d. However, most of the tumor responses were partial regressions. Most encouragingly, the 10.0 μCi/mm³ combination therapy group demonstrated a 100% ORR whereby all tumors vanished 14-21 days after treatment. The median survival for this group was 68.5 d, which was significantly longer than all other combination therapy groups (p<0.05, Mantel-Cox log-rank test). Survival was based completely on humane tumor burden considerations, as no mouse suffered body weight loss greater than the 15% humane threshold (FIG. 8). The overall stability of the ¹³¹I-ELP was found to be equivalent across dose regimens and retain over 70% of the injected dose over 2 decay half-lives of the isotope (FIG. 9).

TABLE 2

| | Tumor | Body | | ¹³¹I-ELP | | | Median | | |
|---|---|---|---|---|---|---|---|---|---|
| Treatment | Size | Wt | | Dose | CP-PTX | CP-PTX | Survival | % | % |
| Group | (mm³) | (g) | n | (μCi/mg) | (mg/kg) | Frequency | (d) | PR | CR |
| High Dose ¹³¹I-ELP + CP-PTX | 134.7 ± 21.8 | 23.1 ± 2.0 | 4 | 9.25 | 25 | 1× Bolus | 68.5 | 0.0 | 100.0 |
| High Dose ¹³¹I-ELP only | 160.4 ± 26.0 | 25.6 ± 1.2 | 5 | 8.13 | n/a | n/a | 31.0 | 20.0 | 20.0 |
| Med. Dose ¹³¹ELP + CP-PTX | 148.2 ± 39.5 | 26.3 ± 2.6 | 5 | 5.97 | 25 | 1× Bolus | 53.0 | 60.0 | 20.0 |
| Med. Dose ¹³¹I-ELP only | 130.4 ± 24.9 | 25.3 ± 3.2 | 5 | 5.71 | n/a | n/a | 33.0 | 0.0 | 0.0 |
| Low Dose ¹³¹I-ELP + CP-PTX | 124.5 ± 22.7 | 27.7 ± 1.6 | 5 | 2.57 | 25 | 1× Bolus | 39.0 | 40.0 | 0.0 |
| Low Dose ¹³¹I-ELP only | 118.4 ± 23.5 | 24.2 ± 1.2 | 5 | 2.30 | n/a | n/a | 24.0 | 0.0 | 0.0 |
| CP-PTX only | 116.4 ± 16.0 | 25.3 ± 1.0 | 4 | n/a | 25 | 1× Bolus | 19.0 | 0.0 | 0.0 |
| Untreated Tumors | 134.8 ± 14.1 | 25.7 ± 1.6 | 4 | n/a | n/a | n/a | 19.0 | 0.0 | 0.0 |

The effect of paclitaxel dose on tumor regression with a constant brachytherapy dose was investigated next. The CP-PTX dose was varied between 12.5, 25, and 50 mg/kg of paclitaxel equivalent, while ¹³¹I-ELP was maintained at 3.3 μCi/mm³ for combination therapy to best differentiate differences between synergistic responses (Table 3). CP-PTX chemotherapy alone, at any dose, had no effect on slowing tumor growth. Combining CP-PTX with the 3.3 μCi/mm³ brachytherapy again inhibited tumor growth (FIG. 4E) but varying the CP-PTX dose produced no significant change in BxPc3 response (p=0.7074, 2-way repeated measures ANOVA).

TABLE 3

| | | Body | | | | | | | |
|---|---|---|---|---|---|---|---|---|---|
| Treatment | Tumor Size | Wt | | ¹³¹I-ELP | CP-PTX | CP-PTX | Median | % | % |
| Group | (mm³) | (g) | n | Dose | (mg/kg) | Frequency | Survival (d) | PR | CR |
| Combotherapy - | 105.2 ± 33.2 | 27.1 ± 2.3 | 5 | 2.96 uCi/mg | 50 mg/kg | 1× Bolus | 41.0 | 40.0% | 0.0% |
| Full MTD CP-PTX | | | | | | | | | |
| Full MTD CP-PTX only | 82.7 ± 23.2 | 27.2 ± 2.7 | 5 | n/a | 50 mg/kg | 1× Bolus | 25.0 | 0.0% | 0.0% |
| Combotherapy - | 98.0 ± 30.9 | 28.2 ± 1.3 | 5 | 2.79 uCi/mg | 25 mg/kg | 1× Bolus | 36.0 | 0.0% | 0.0% |
| Half MTD CP-PTX | | | | | | | | | |
| Half MTD CP-PTX only | 118.4 ± 4.3 | 30.7 ± 1.6 | 4 | n/a | 25 mg/kg | 1× Bolus | 25.0 | 0.0% | 0.0% |
| Combotherapy - | 110.4 ± 54.7 | 29.4 ± 1.2 | 4 | 2.84 uCi/mg | 12.5 mg/kg | 1× Bolus | 67.0 | 25.0% | 0.0% |
| Quarter MTD CP-PTX | | | | | | | | | |
| Quarter MTD CP-PTX only | 123.1 ± 10.6 | 28.3 ± 2.7 | 6 | n/a | 12.5 mg/kg | 1× Bolus | 21.0 | 0.0% | 0.0% |
| 2× 25 mg/kg | 108.7 ± 34.6 | 28.3 ± 2.4 | 5 | 3.04 uCi/mg | 25 mg/kg | 2×, q.w. | 41.0 | 0.0% | 0.0% |
| Combotherapy | | | | | | | | | |
| 2× 25 mg/kg | 112.7 ± 44.6 | 27.4 ± 3.3 | 5 | n/a | 25 mg/kg | 2×, q.w. | 23.0 | 0.0% | 0.0% |
| CP-PTX only | | | | | | | | | |
| ¹³¹I-ELP only | 119.3 ± 20.5 | 27.9 ± 2.3 | 5 | 2.82 uCi/mg | n/a | n/a | 29.0 | 20.0% | 0.0% |
| Untreated Tumors | 110.8 ± 25.7 | 28.4 ± 2.1 | 4 | n/a | n/a | n/a | 21.0 | 0.0% | 0.0% |

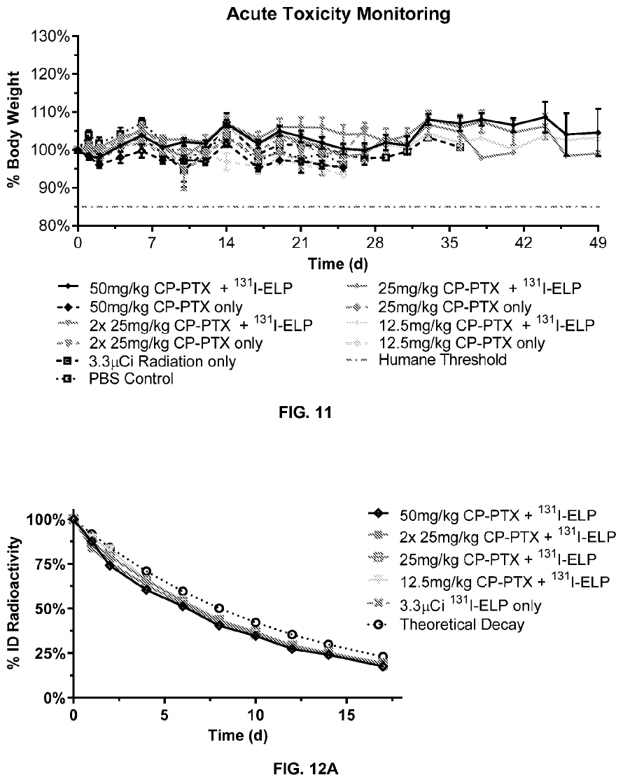

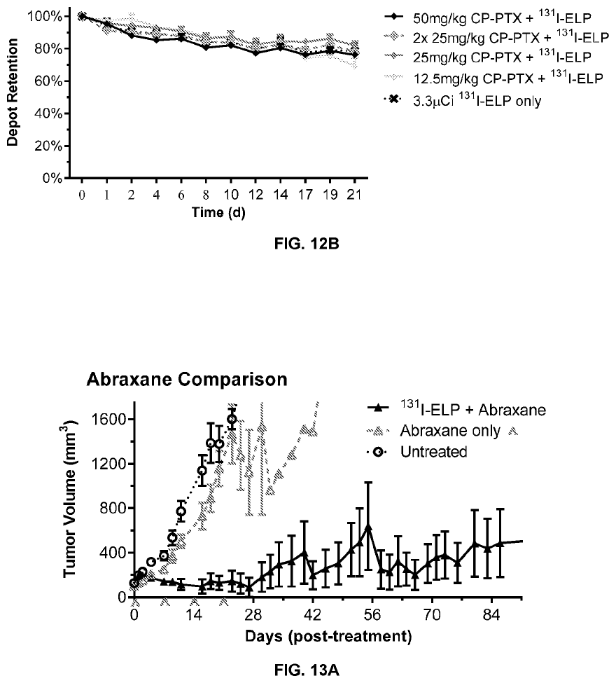

Survival (FIG. 4F, FIG. 10) for all combination doses was significantly improved over the 21 d survival median observed in untreated mice (p<0.05, Mantel-Cox log-rank test). The 12.5 mg/kg combination treatment achieved the highest median survival at 67 d, compared to 36 d and 41 d for 25 mg/kg and 50 mg/kg respectively (P=0.1548, Mantel-Cox log-rank test). Interestingly, when a second injection of CP-PTX at 25 mg/kg was administered one week after initial combination treatment (FIG. 4G), the tumor response was significantly improved over combination therapy receiving a single CP-PTX injection (P=0.0013, 2-way ANOVA). Survival was modestly improved from 36 d to 41 d (FIG. 4H, p=0.0286, log-rank Mantel Cox). Finally, no signs of acute toxicity (FIG. 11) or dissolution of the ¹³¹I-ELP depot (FIG. 12) was observed under any CP-PTX dosing conditions. This suggested that repeated CP-PTX injections could extend the duration of the therapeutic effect, even if the effect was not heavily influenced by the paclitaxel dose.

From these experiments, an optimized dose regimen for combination therapy was determined, with 10.0 μCi/mm³ of ¹³¹I-ELP selected as the most efficacious radioactivity dose. For CP-PTX, four, once-weekly injections of CP-PTX at a dose of 12.5 mg/kg of paclitaxel equivalent was selected as it would minimize individual drug dose and potential systemic toxicity. This duration was selected to ensure synergistic interaction for four effective half-lives of ¹³¹I-ELP radioactivity.

### Example 3: Comparative Efficacy with Current Clinical Therapies

The dose escalation studies showed promise that the combination strategy could prove efficacious against pancreatic tumors. Next, the same combination strategy was validated with the current clinical standards of care. The response of subcutaneous BxPc3 tumors to combination therapy were re-examined with CP-PTX replaced by Abraxane and ¹³¹I-ELP brachytherapy replaced with EBRT.

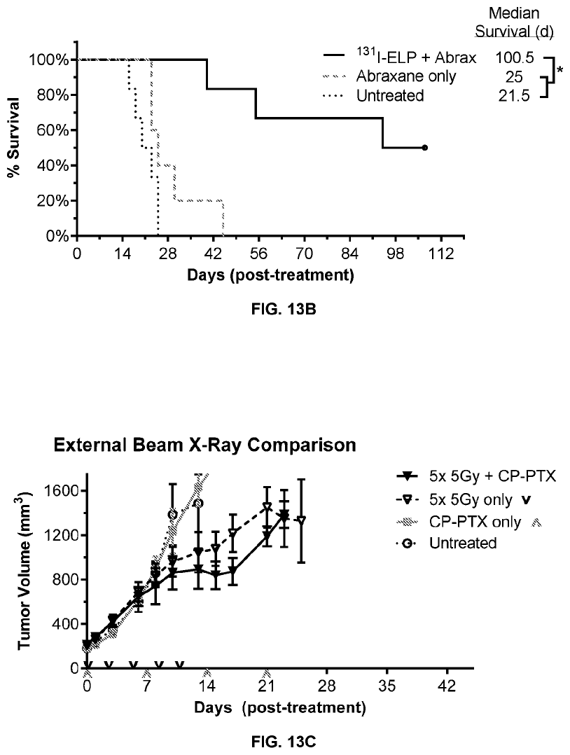

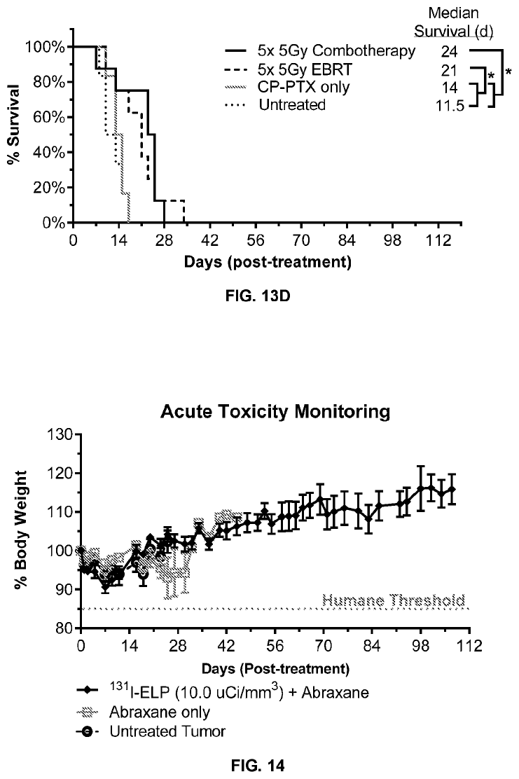

As shown in FIG. 13A, Abraxane administered weekly at 12.5 mg/kg for four weeks produced a minimal effect on BxPc3 tumor growth (n=6) as a stand-alone treatment. Only one mouse exhibited a partial response (Table 4). The median survival for Abraxane monotherapy was 25 d compared to 21.5 d for untreated tumors (p=0.0848, log-rank test). When Abraxane was combined with ¹³¹I-ELP brachytherapy at 10 μCi/mm³, BxPc3-luc2 tumors demonstrated significant regression (p<0.0001, 2-way repeated measures ANOVA). A 83.3% ORR was achieved in accordance with the RECIST criteria, with 5/6 mice achieving complete responses. The Kaplan-Meier analysis in FIG. 13B shows that the respective median survival was 100.5 d, quadrupling the survival of Abraxane monotherapy. The maximum tumor responses exhibited by Abraxane combination therapy were not statistically different compared to those achieved with CP-PTX in FIG. 4 (p=0.4704, unpaired t-test), clearly showing that the formulation of paclitaxel was not critical to the observed therapeutic effect. No signs of acute toxicity (FIG. 14) or dissolution of the ¹³¹I-ELP depot (FIG. 15) were observed under any Abraxane dosing conditions.

TABLE 4

| | Tumor | Body | | | | | Median | | |
|---|---|---|---|---|---|---|---|---|---|
| Treatment | Size | Wt | | X-ray | | CP-PTX | Survival | % | % |
| Group | (mm³) | (g) | n | Dose | CP-PTX | Frequency | (d) | PR | CR |
| X-ray EBRT | 210.8 ± 66.9 | 27.2 ± 1.7 | 8 | 5× 5 Gy | 12.5 mg/kg | 4×, q.w. | 24.0 | 0.0 | 0.0 |
| Combotherapy | | | | | | | | | |
| X-ray EBRT only | 208.5 ± 62.8 | 28.0 ± 2.3 | 8 | 5× 5 Gy | n/a | n/a | 21.0 | 0.0 | 0.0 |
| CP-PTX only | 183.1 ± 38.0 | 27.8 ± 1.1 | 6 | n/a | 12.5 mg/kg | 4×, q.w. | 13.0 | 0.0 | 0.0 |
| Untreated Control | 180.0 ± 53.5 | 28.6 ± 2.4 | 6 | n/a | n/a | n/a | 11.5 | 0.0 | 0.0 |

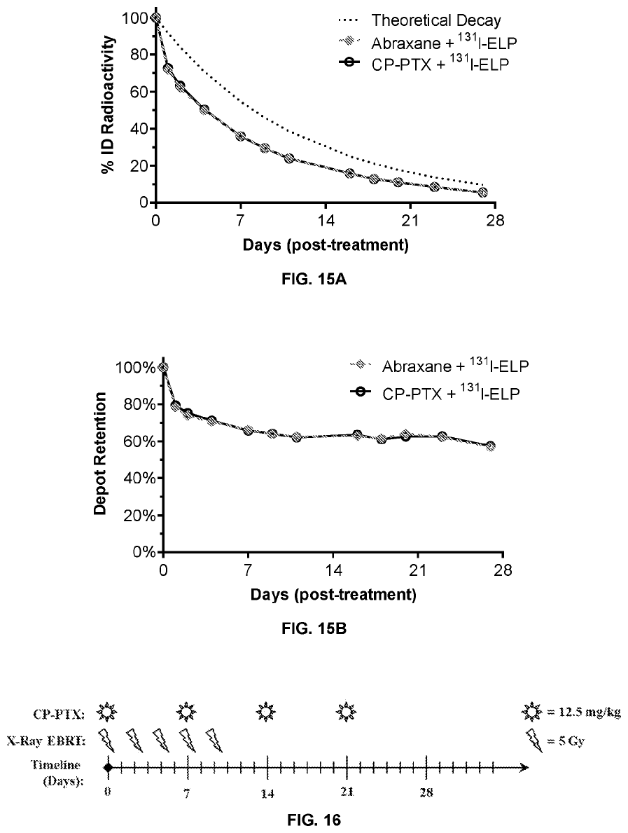

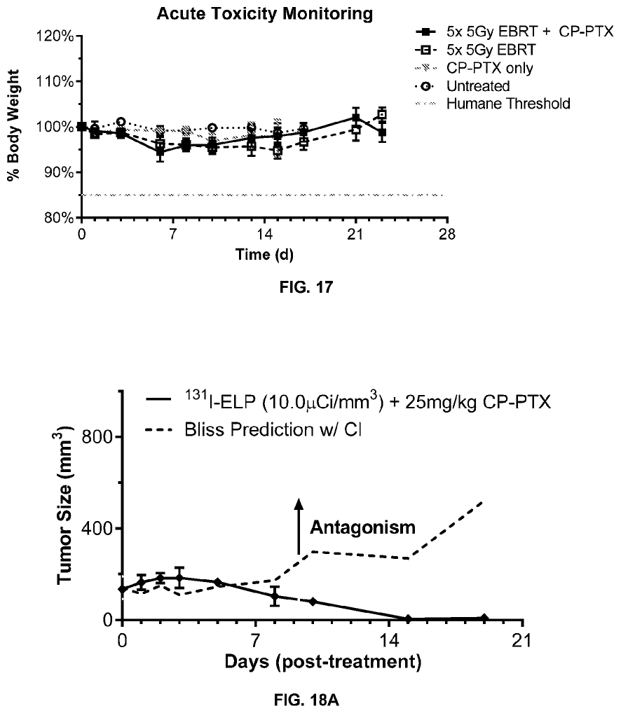

The importance of ¹³¹I-ELP brachytherapy on treatment outcome by replacing it with the current radiation oncology standard of care—EBRT. A 25 Gy hypofractionated regimen was selected to mimic current clinical approaches for treating pancreatic cancer. BxPc3 tumors were treated with 5 Gy fractions every other day delivered from a micro-irradiator, while CP-PTX was i.v. injected at 12.5 mg/kg on day 0 and 7 (FIG. 16). CP-PTX injections were administered 30 min prior to EBRT treatment. FIG. 13C shows that EBRT only produced a modest inhibition of overall tumor growth. Interestingly, combining once weekly CP-PTX chemotherapy at 12.5 mg/kg with EBRT did not improve BxPc3-luc2 regression over EBRT alone (FIG. 13C, p=0.6616, 2-way repeated measures ANOVA). No survival advantage was seen between mice treated with EBRT combination therapy survived for 24 d compared to 21 d for EBRT-only treatment (FIG. 13D, Table 5). No signs of acute toxicity (FIG. 17) were observed. These results stand in stark contrast to the tumor responses observed in for ¹³¹I-ELP brachytherapy, where combination with CP-PTX chemotherapy resulted in the complete ablation of tumors.

TABLE 5

| | Tumor | Body | | | | | Median | | |
|---|---|---|---|---|---|---|---|---|---|
| Treatment | Size | Wt | | X-ray | CP-PTX | CP-PTX | Survival | % | % |
| Group | (mm3) | (g) | n | Dose | (mg/kg) | Frequency | (d) | PR | CR |
| X-ray EBRT | 210.8 ± 66.9 | 27.2 ± 1.7 | 8 | 5× 5 Gy | 12.5 | 4×, q.w. | 24.0 | 0.0 | 0.0 |
| Combotherapy | | | | | | | | | |
| X-ray EBRT only | 208.5 ± 62.8 | 28.0 ± 2.3 | 8 | 5× 5 Gy | n/a | n/a | 21.0 | 0.0 | 0.0 |
| CP-PTX only | 183.1 ± 38.0 | 27.8 ± 1.1 | 6 | n/a | 12.5 | 4×, q.w. | 13.0 | 0.0 | 0.0 |
| Untreated Control | 180.0 ± 53.5 | 28.6 ± 2.4 | 6 | n/a | n/a | n/a | 11.5 | 0.0 | 0.0 |

### Example 4: Synergy Analysis with the Bliss Independence Framework

The tumor regression data were then analyzed by the Bliss Independence framework to assess whether combining ¹³¹I-ELP treatment with paclitaxel (formulated as either CP-PTX or Abraxane) produced mathematically demonstrable synergy. The Bliss Independence framework is a probabilistic method to analyze the non-linear independence of therapeutic agents without requiring full characterization of the dose-response spectrum—an ideal method for in vivo tumor regression data. Briefly, the Bliss model supposes the null case where the combined effect of independent agents can be predicted by the product of the ‘fraction of effect’ achieved by each agent as a monotherapy. This case can be simplified to the 2-agent case, where the ‘fraction of effect’ would be the measurable tumor volume remaining after treatment. Therefore, among various ABDs, only ABDs that do not lower the transition temperature of the CP-drug conjugate to 40° C. or lower will be useful for preparing albumin binding nanoparticulate systems.

f_{Bliss Predicted}=f₁f₂

The observed response is then compared to the model prediction to determine whether agents are synergistic, independent, or antagonistic, as follows:

$${f}_{Observed} \{ \begin{matrix}> {f}_{Predicted} , Bliss Antagonism \\ = {f}_{Predicted} , Independent Mechanisms \\ < {f}_{Predicted} , Bliss Synergy\end{matrix}$$

Due to the probabilistic underpinning of the Bliss Model, the significance of the observed effects can be statistically evaluated using parametric analysis. The standard deviation of the predicted response is given by the formula,

σ_{Bliss Predicted}=\sqrt{E(f₁²)E(f₂²)−E(f₁)²E(f₂)²},

a derivation of which is contained in Example 9.

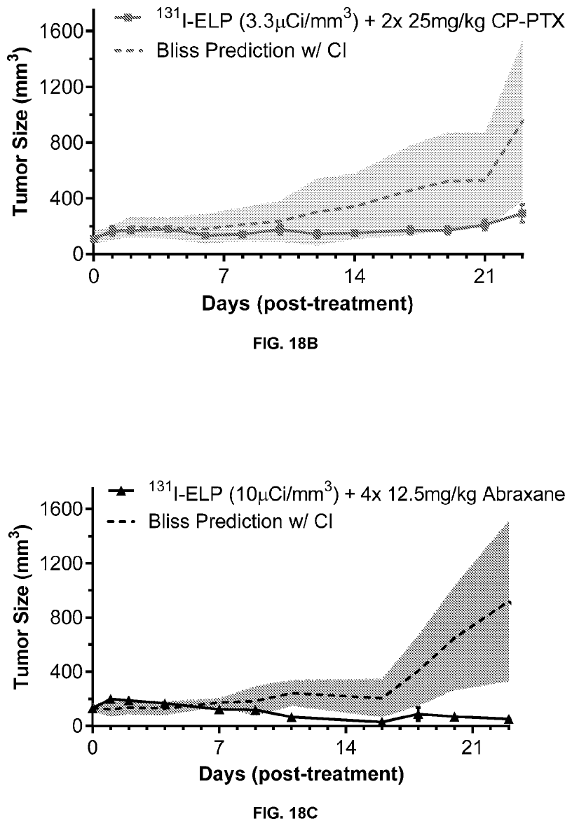

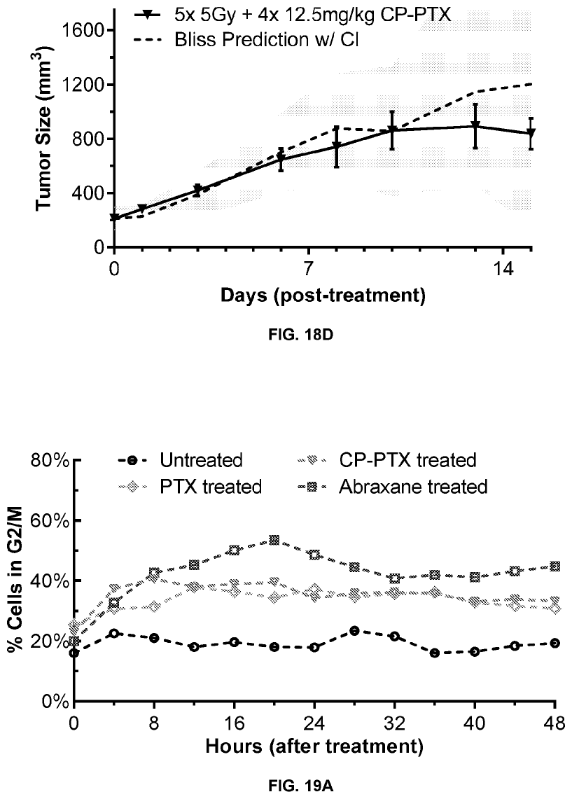

This framework was then used to analyze the regression studies from FIG. 4 and FIG. 13, resulting in the Bliss isobolograms shown in FIG. 18. The experimentally measured tumor responses to tumor therapy (f_{Observed}) are shown as a solid line while Bliss predictions are graphically represented as dashed lines with shaded 95% confidence intervals. When tumor regression was evaluated in the radioactivity dose-escalation study (FIG. 18A), the observed tumor regression significantly exceeded the Bliss predicted value (p<0.0001, repeated measure 2-way ANOVA). For the trials examining multi-dose CP-PTX and Abraxane combination therapy, Bliss analysis indicated that the interaction between ¹³¹I-ELP and the two paclitaxel formulations was synergistic in both cases (p<0.001). The same analysis for combination treatment with EBRT and CP-PTX nanoparticles, however, demonstrated no synergy, as the tumor response nearly matched the predicted Bliss response (p=0.7472).

### Example 5: Enhanced Cytotoxicity by Cellular Radiation Sensitization

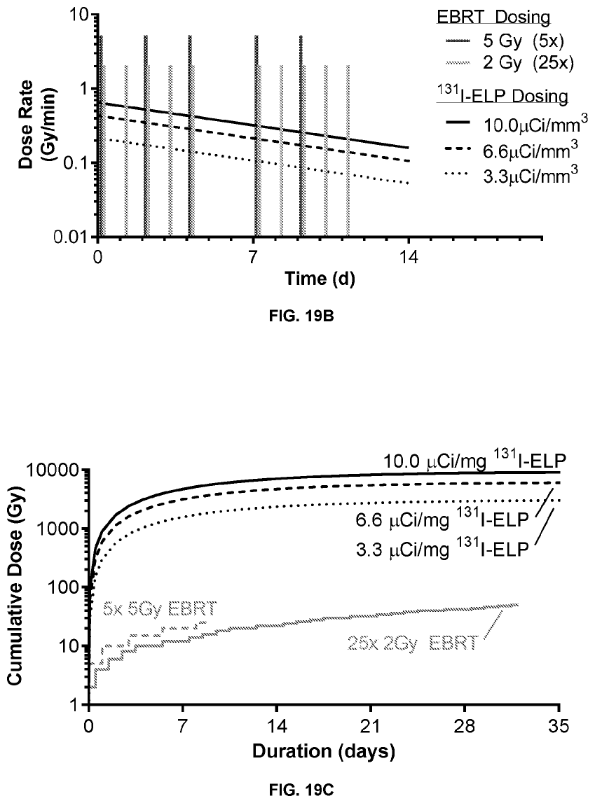

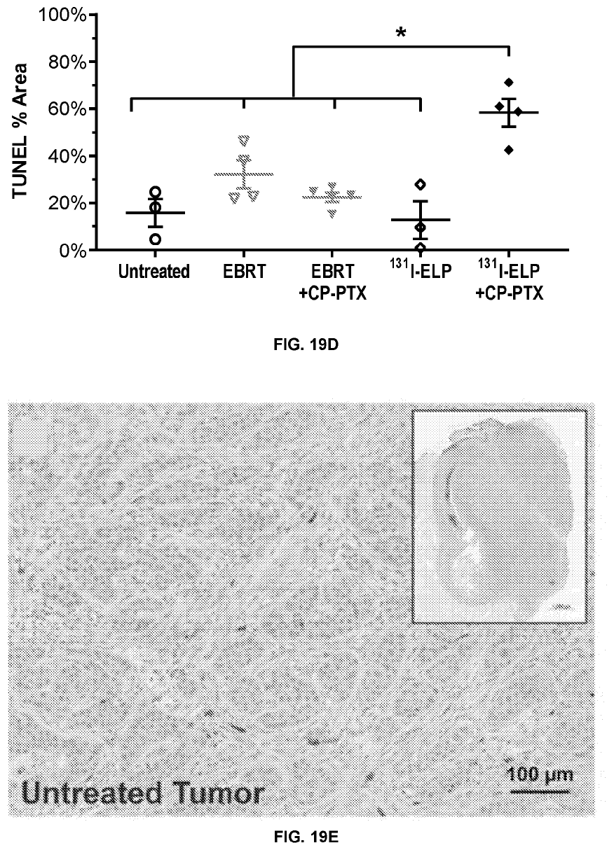

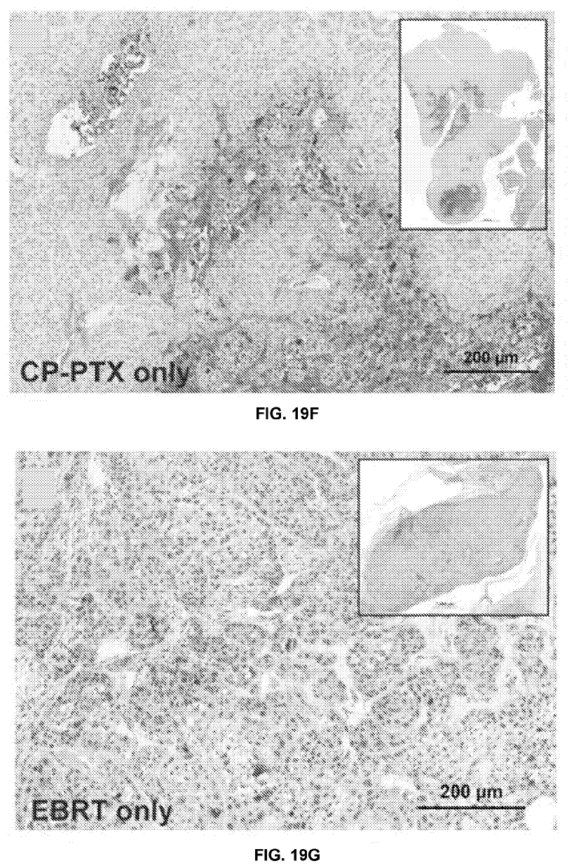

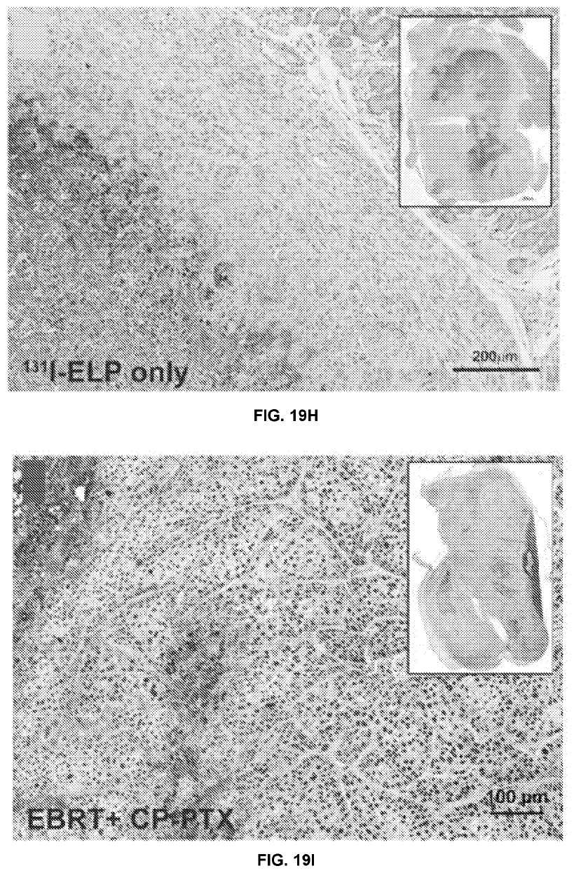

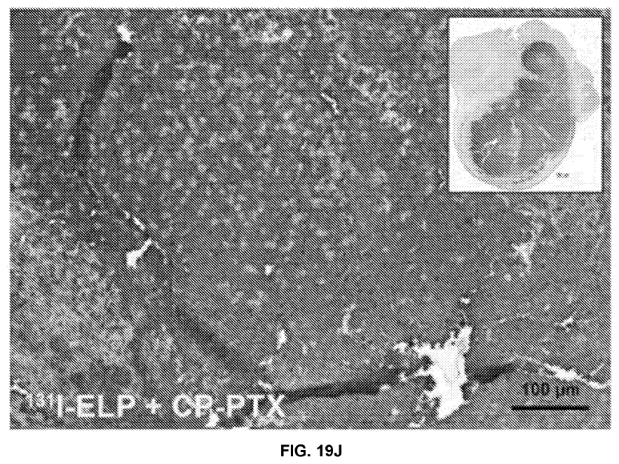

Experiments were undertaken next to determine why the systemic treatment with paclitaxel produced such profound anti-tumor synergy when combined with ¹³¹I-ELP brachytherapy but not with EBRT. As radiation sensitization is driven by late G2/M arrest, the effects of paclitaxel on cell cycle progression were examined. BxPc3 cells were treated in vitro with 1 nM of equivalent paclitaxel, stained with propidium iodide to measure relative DNA quantities, and analyzed by flow cytometry to assess the cell cycle distribution over time. All paclitaxel formulations showed that the proportion of cells entering the radiation sensitive G2/M phase increased gradually over time (FIG. 19A). For free PTX and CP-PTX treated cells, the maximum proportion of sensitized cells was reached after 12 hours, peaking at 37.9% and 40.5% of the cell population, respectively. Only 18.0% of untreated cells, by comparison, were similarly in the G2/M phase. Abraxane treated cells meanwhile, reached a peak G2/M distribution of 50.1% after 16 h, but quickly receded afterwards. In all cases, the majority of cells typically remained in highly resistant G1 and S phases, where upregulation of non-homologous end-joining and base excision repair mechanisms are highly active in repairing DNA damage caused by ionizing radiation.

When this profile of tumor cell sensitization was compared to the radiation exposure profile of ¹³¹I-ELP brachytherapy and EBRT (FIG. 19B), the advantage of ¹³¹I-ELP becomes apparent. X-ray EBRT is only applied for minutes; allowing many tumor cells to survive treatment while in resistant cell-cycle phases. The continuity of the β-radiation provided by intratumoral ¹³¹I-ELP depot, however, ensures that all tumor cells are exposed to treatment as they eventually progress into the radiation vulnerable late G2/M phase. Using MIRD formalisms for ¹³¹Iodine decay emissions, a dose rate of 0.647 Gy/min was calculated for ¹³¹I-ELP brachytherapy (see Example 11). This was found to be lower than the 5 Gy/min of X-ray EBRT, but the ¹³¹I-ELP compensates for a lower dose rate by constantly irradiating the tumor. Using Siegel & Stabin's model for scaled absorption spheres of beta particles coupled with the continuous stopping distance approximation (CSDA) for electrons at respective ¹³¹Iodine energies, over 98.1% of the ¹³¹I-ELP β-particles are absorbed within the margin of the tumor. This results in a cumulative delivered dose that is more than 100-fold higher than EBRT (FIG. 19C). The remaining J-particles are absorbed within 2 mm of the tumor.

These differences between X-ray EBRT and ¹³¹I-ELP brachytherapy in dose rate and temporal coordination with the effects of paclitaxel next led us to examine how in vivo cytotoxicity was affected by each radiation modality. Athymic nu/nu mice were orthotopically implanted with BxPc3-luc2 tumors (n=3-4) and received i.v. CP-PTX (25 mg/kg), X-ray EBRT (5×5 Gy), ¹³¹I-ELP brachytherapy (10 μCi/mm³), or a combination thereof. After 12 days, tumors were removed and processed for TUNEL staining to evaluate apoptosis. CP-PTX resulted in focal areas of apoptosis (FIG. 19F) with the majority of the tumor appearing similar to the untreated controls (FIG. 19E). EBRT induced a low intensity, homogenous level of TUNEL staining in the BxPc3 tumors (FIG. 19G). In contrast, ¹³¹I-ELP brachytherapy resulted in a higher level of positive TUNEL staining that appeared spatially confined within the tumor margins (FIG. 19H). Tumor sections from mice treated with EBRT and CP-PTX chemotherapy displayed a positive TUNEL staining pattern that looked distinctively like a merging of the two respective monotherapies (FIG. 19I). Diffuse, homogenous nuclear staining was evident throughout the bulk of the tumor with sporadic patches of stronger staining, as if combining therapies provided an additive apoptotic response. In a marked contrast to all other treatment groups, the combination of ¹³¹I-ELP with CP-PTX exhibited large areas of intense TUNEL staining in tumor specimens (FIG. 19J). Few viable cells could be identified due to the intensity of the staining, which was dramatically stronger than either monotherapy alone and indicative of increased apoptosis for the combination therapy. The regions of apoptosis were also more extensive for the ¹³¹I-ELP and CP-PTX combination therapy than for ¹³¹I-ELP monotherapy specimens. The fractional cross-sectional areas of tumor apoptosis were quantified using a binary mask of TUNEL staining for all treatment groups (FIG. 19D). Tumors treated with ¹³¹I-ELP and CP-PTX combination therapy exceeded all other treatment groups in total area (p=0.0006, ANOVA). These results could arise from two potential phenomena: first, that paclitaxel-mediated sensitization in the tumor increases both the effective range and intensity of ¹³¹I-ELP brachytherapy cytotoxicity; and second, that ¹³¹I-ELP could improve the diffusion of paclitaxel throughout the tumor and allow it to better contribute to cell killing.

### Example 6: Effect of β-Brachytherapy on the Tumor Microenvironment

In addition to amplifying the cytotoxic response within pancreatic cancer cells, it was speculated that continuous exposure to β-radiation from ¹³¹I-ELP might induce substantial changes in the tumor microenvironment. The unique tumor microenvironment is a defining feature of pancreatic cancer and is widely attributed as a primary cause of its resistance to conventional therapies. In addition to their hypovascularity and dense stromal content, pancreatic tumors also upregulate the expression of a number of junction proteins that act as biological barriers to inhibit the penetration and retention of chemotherapeutics. Reducing these barriers has attracted interest to improve oncology therapeutics. The pathological integrity of these barriers was explored in an orthotopic BxPc3-luc2 tumor model after receiving either ¹³¹I-ELP (10.0 μCi/mm³) or EBRT (5×5 Gy) in combination with weekly CP-PTX (12.5 mg/kg). After 12 days, tumor specimens were randomized, blinded, and then analyzed for immunohistochemical markers by a board-certified pathologist.

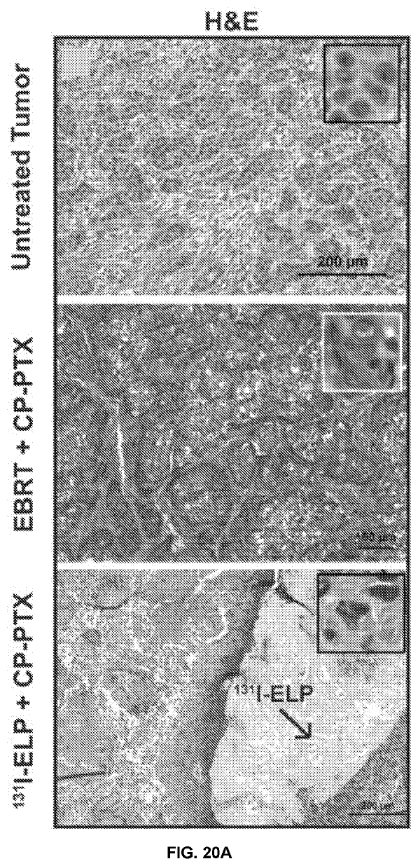

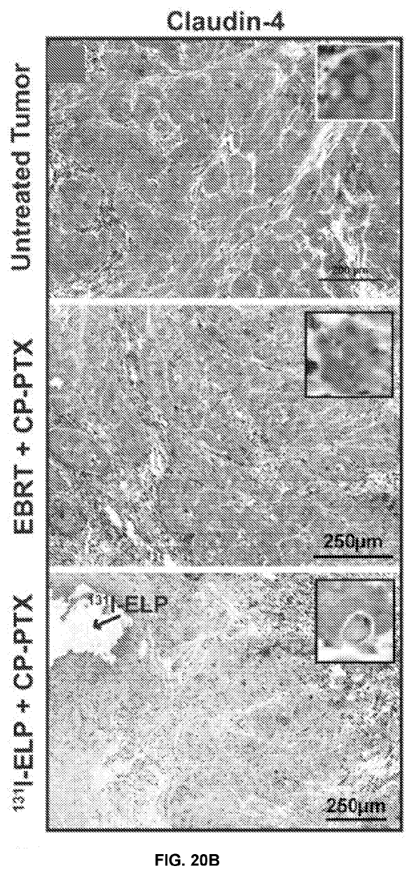

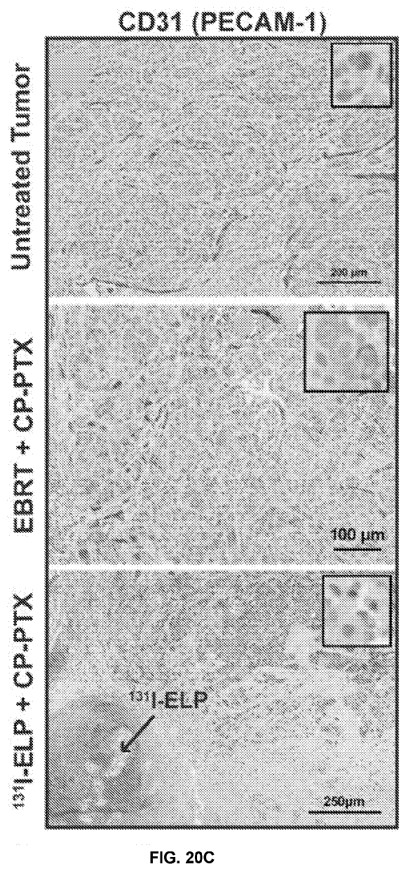

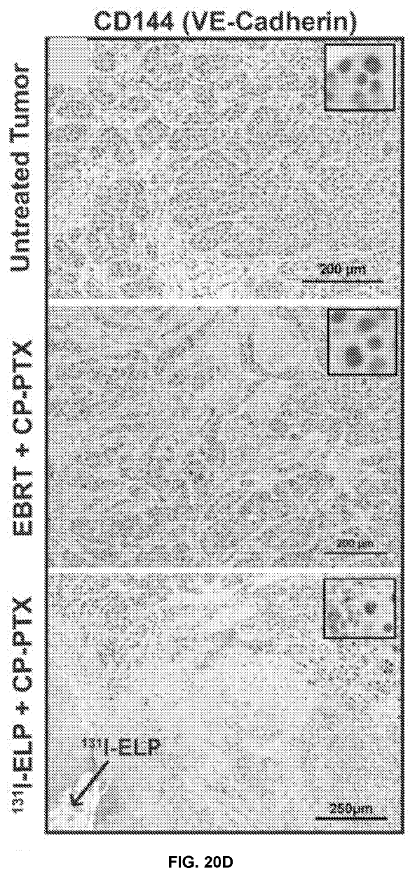

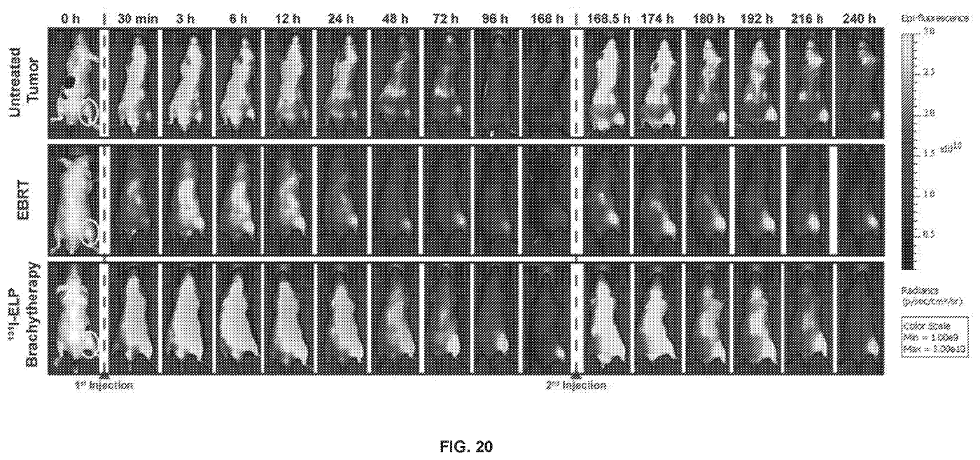

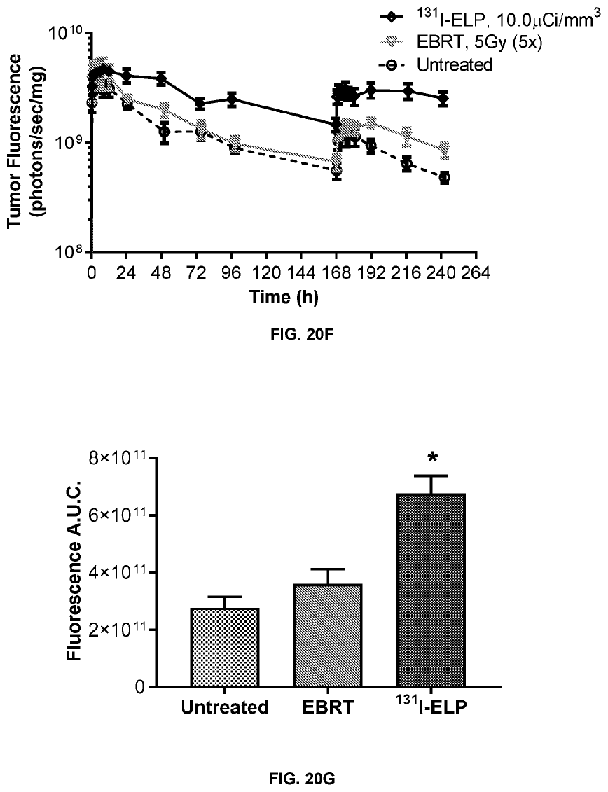

Tumor histology was evaluated by H&E staining (FIG. 20A). The adenocarcinomas are rich in fibrous stroma, with areas of necrosis even in untreated tumors. Tumors treated with a combination of X-ray EBRT and CP-PTX exhibited focal necrosis (˜20%) and had more prominent stromal fibrosis and inflammation than untreated controls. In comparison, tumors treated with ¹³¹I-ELP and CP-PTX combination therapy had far larger large areas of necrosis and tumor cells with nuclear pleomorphism. Gelatinous acellular debris, consistent with ELP depots, was visible in the center of necrotic areas. These features were similar to, but far more pronounced, in tumors treated with ¹³¹I-ELP monotherapy. Tumor sections were additionally stained with Masson Trichrome (See Example 12) to further examine the integrity of the stromal collagen. A blinded histologic review revealed minimal-to-moderate amounts of stromal collagen in ¹³¹I-ELP treated tumors. EBRT appeared to induce the opposite effect, with tumors exhibiting dense stromal collagen.

Further immunohistochemical (IHC) staining with anti-Claudin 4, CD-31, and CD-144 was also performed. Claudin-4, a tight junction protein that regulates paracellular diffusion, was present in BxPc3-luc2 pancreatic tumor cells with intense staining in the cytoplasm and cell membrane (FIG. 20B). Its expression was not altered upon EBRT and CP-PTX combination therapy. However, tumors treated with ¹³¹I-ELP and CP-PTX demonstrated reduced Claudin-4 expression in tumor cells near the ELP depots. When the relative area of tumor expression was quantified as a binary mask in ImageJ, Claudin-4 levels were found to be significantly reduced for tumors treated with ¹³¹I-ELP brachytherapy (p<0.05, see Example 12).

This effect was specific for Claudin-4 and did not extend to the two other junction proteins investigated. CD-31, also known as platelet endothelial cell adhesion molecule 1 (PECAM-1), was examined as it is a vasculature marker that can be upregulated in cancer to promote angiogenicity and drug resistance. Radiation therapy has been indicated to induce PECAM-1 over-expression in tumors. However, tumors treated with ¹³¹I-ELP combination therapy exhibited staining patterns and intensities similar to untreated tumors (FIG. 20C). No significant difference in tumor expression was observed in treated or untreated specimens (p>0.6569, one-way ANOVA and post-hoc Tukey test), nor in monotherapy treated controls (see Example 12).

The expression of the adherens junction glycoprotein VE-cadherin (CD-144), which supports the vascular endothelial barrier, and is commonly expressed in a variety of solid tumors, was studied. IHC staining confirmed CD-144 expression in untreated BxPc3 pancreatic tumor cells (FIG. 20D) with weak cytoplasmic, but strong nuclear expression. No differences were observed in EBRT treated tumors, with or without CP-PTX (see Example 12). Tumors treated with ¹³¹I-ELP brachytherapy had heterogeneous expression of CD-144. Regions proximal to the ¹³¹I-ELP appeared weakly stained with disrupted patterning. Areas of the tumor distant from the ¹³¹I-ELP sites resembled expression patterns seen in untreated tumors. A decrease in nuclear VE-Cadherin staining was confirmed after ¹³¹I-ELP treatment, but the overall coverage was not significantly different from untreated tumors (p=0.2318, one-way ANOVA).

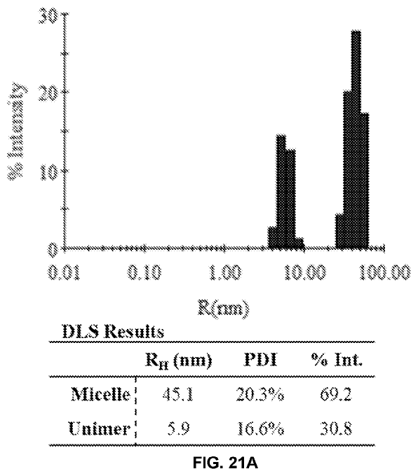

These pathological changes in the tumor microenvironment suggested that the biological barriers regulating tumor permeability were substantially weakened by ¹³¹I-ELP brachytherapy. The impact of these changes, however, could not be assessed from histology alone. Radiation treatment induced changes in tumor uptake and retention of chemotherapy agents were directly examined. CP-PTX was fluorescently labeled with the near-infrared fluorophore sulfoCy5.5 to enable in vivo imaging using the IVIS Lumina XR. SulfoCy5.5 was selected for several reasons: (1) its near infrared emission wavelength of 710 nm should minimize tissue attenuation of the fluorescence signal. (2) The sulfonated variant of Cy5.5 was selected for its hydrophilicity, as Cy5.5 is hydrophobic in aqueous solution. Negligible change in micelle size was observed after labeling using dynamic light scattering (FIG. 21A). SulfoCy5.5-CP-PTX retained its nanoparticle size with an RH of 45.1 and tolerable monodispersity, albeit slightly larger than the original CP-PTX micelles of 38.2 nm. Interestingly, SLS was very hard to run due to the spectral overlap of Cy5.5 with the SLS laser. The fluorophore emissions upon excitation created a high background level that the SLS misinterpreted as scattering results. The fluorophore had to be photo bleached before a meaningful SLS data could be gathered (FIG. 21B). These results indicated an RG of 53.7 nm. Taken together, the fluorescently labeled particles were could be assured to reasonably approximate their CP-PTX counterpart in size and diffusive behavior.

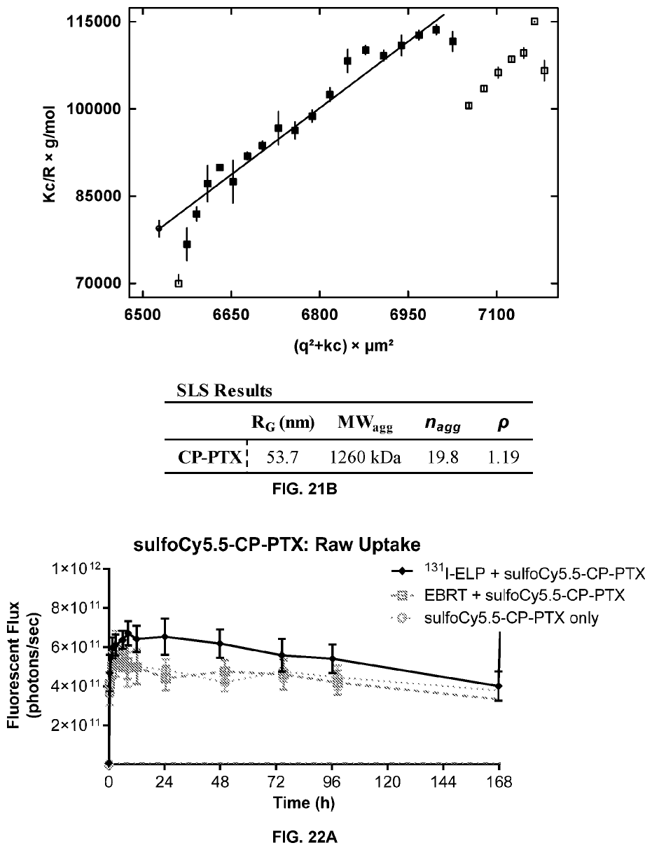

Relative permeability of the tumor was then measured by quantifying tumor-specific uptake of the sulfoCy5.5-CP-PTX nanoparticles over time in subcutaneous BxPc3 tumors (FIG. 20E and FIG. 22). Fluorescent flux was normalized to tumor size and revealed a significant increase in tumor-uptake of the fluorescent for ¹³¹I-ELP treated tumors compared to both EBRT and untreated tumors (FIG. 20F, p<0.05, repeated measure ANOVA). Post-hoc analysis revealed that X-ray EBRT did not induce higher accumulation of the drug-loaded nanoparticles compared to untreated tumors (p>0.801, Sidak multiple comparisons test). The area under the curve (AUC) was then integrated in FIG. 20G to determine the relative uptake of each treatment. This equated to a 187.9% higher accumulation of CP-PTX in ¹³¹I-ELP treated tumors over EBRT and a 198.5% increase over untreated tumors.

To determine if the paclitaxel was a contributing factor for the increased accumulation, this study was repeated using a soluble ELP polymer conjugated to a fluorophore without any drug

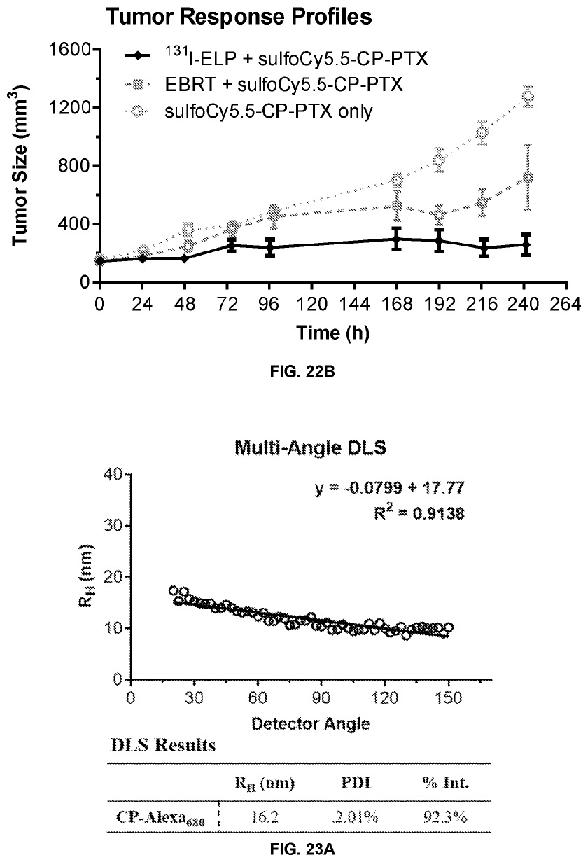

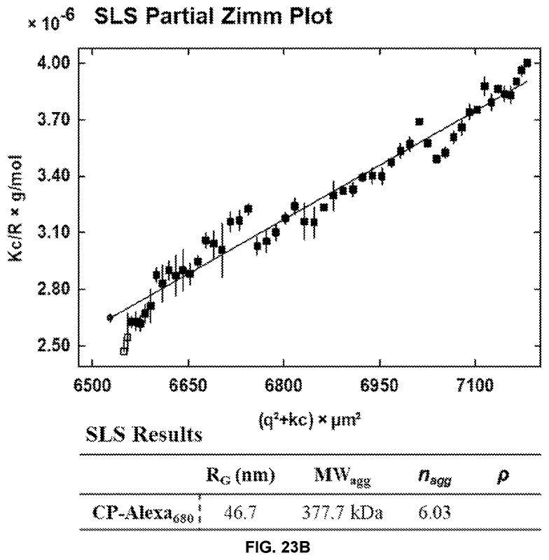

In this study, a NIR fluorophore Alexa680 was utilized. However, it was conjugated to the chimeric ELP without any paclitaxel. Light scattering analysis (FIG. 23) showed that the resulting conjugates did not resemble the previous CP-PTX nanoparticles, being smaller in size and having a different shape factor. In fact, they more closely approximated single polymer ELPs.

Convincingly, the results in FIG. 24 mirrored the previous findings of the sulfoCy5.5-CP-PTX trial. CP-Alexa680 was intravenously administered to mice on the initial day of treatment and a follow up dose was given at 7 days. The resulting fluorescent flux profile showed significantly higher uptake per tumor in the ¹³¹I-ELP treated group (p=0.002 2-way, ANOVA). External beam radiotherapy, however, showed no significant advantages in CP-Alexa680 accumulation compared to untreated controls. Tumors were also treated with sham, ELP-only injections to ensure that physical injections did not alter uptake. Mice in this treatment group resulted in the same accumulation levels as the untreated controls.

The ¹³¹I-ELP accumulation was 170.1% higher than EBRT, 235.6% higher than untreated tumors, and 265.9% higher than tumors injected with a sham intratumoral ELP depot.

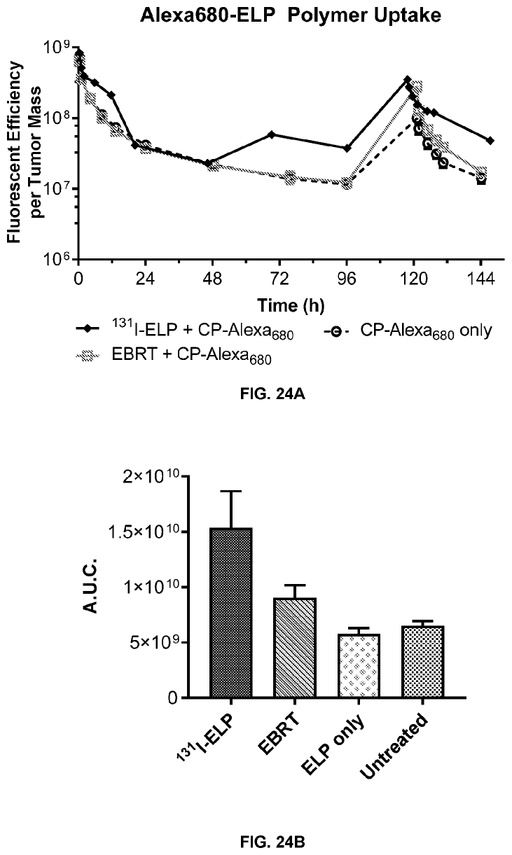

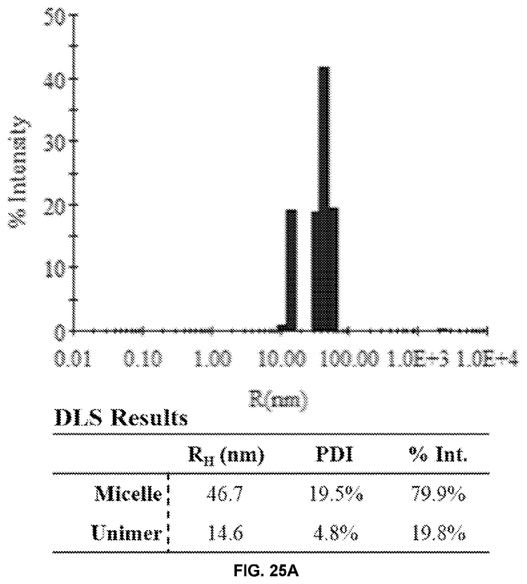

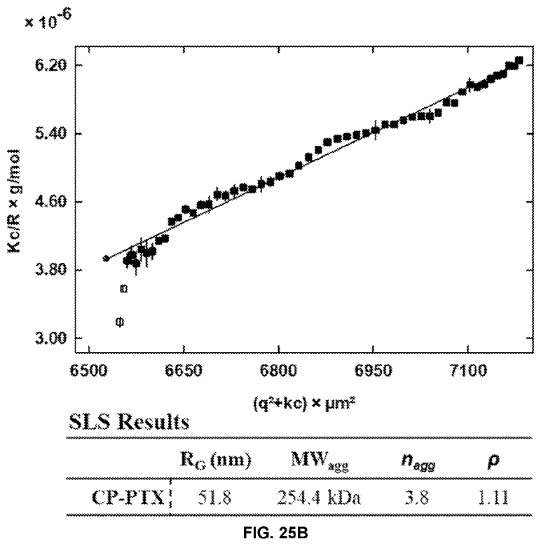

To ensure further validity, a final follow-up study was also conducted to examine the accumulation profile of tumors treated with nanoparticles that resembled CP-PTX size, but lacked paclitaxel. This was achieved using the simplified Cy5.5 fluorophore in maleimide form. As Cy5.5 is a hydrophobic molecule, although not to the same extent as paclitaxel, conjugation to the cysteine domain of CPELP was found to induce nanoparticle self-assembly when examined by light scattering (FIG. 25).

As EBRT treated tumors were consistently found to produce no significant enhancement of tumor perfusion and accumulation in the previous trials, this final study was simplified. Only untreated tumors were compared to ¹³¹I-ELP treatments with a single bolus infusion of CP-Cy5.5. The resulting fluorescent profile (FIG. 26) proved similar to all other trials. ¹³¹I-ELP treated tumors again showed higher fluorescent flux readings over untreated tumors. This was quantified using area under the curve analysis—showing a 159.5% increase in nanoparticle accumulation. These results conclusively showed that the molecular effects of continuous ¹³¹I-ELP irradiation enhanced the penetration and retention of paclitaxel within the tumor.

### Example 7: Efficacy Across Diverse Pancreatic Tumor Genotypes

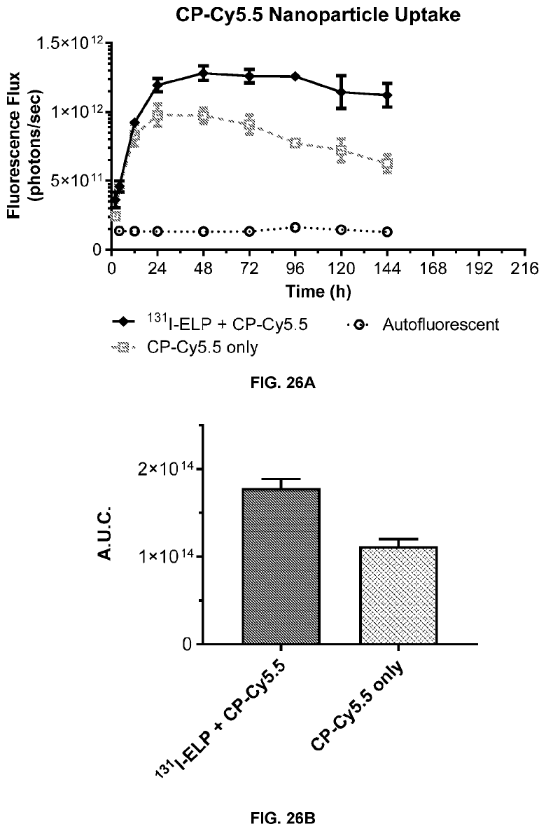

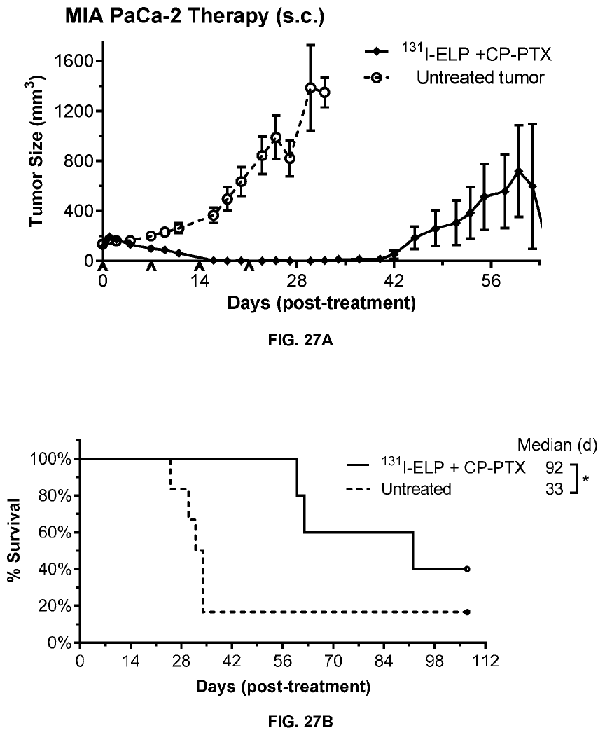

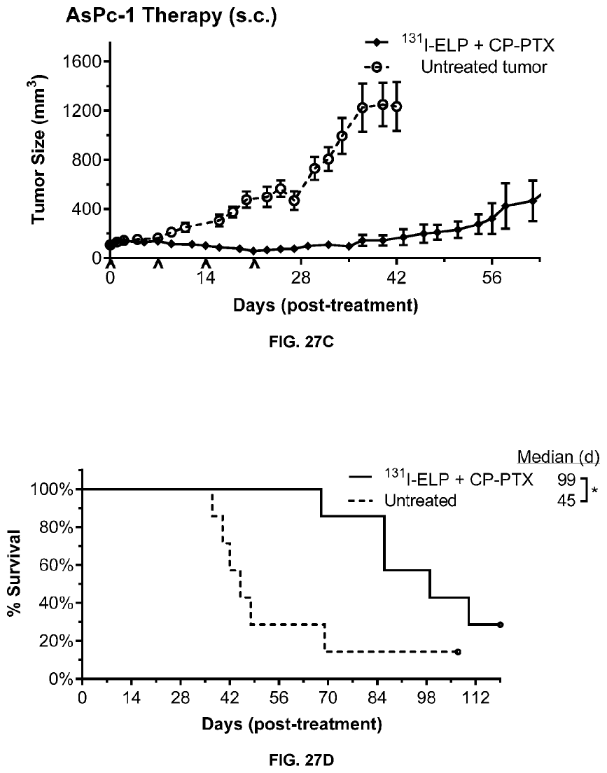

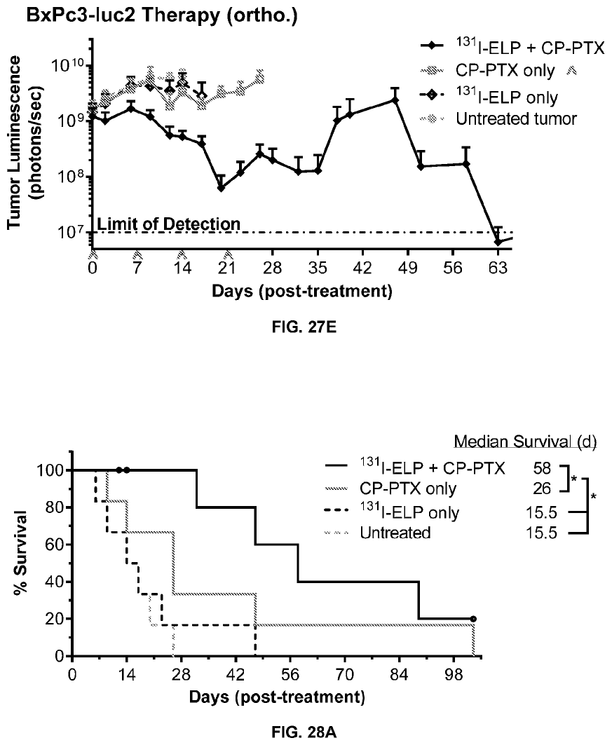

The synergistic potential of the ¹³¹I-ELP combination strategy was explored across multiple pancreatic tumors with diverse genetic and phenotypic makeups. First, the optimized treatment regimen was assessed if it would prove successful against a MIA PaCa-2 subcutaneous tumor model. Unlike BxPc3, the MIA PaCa-2 human tumor cell line has K-ras mutations found commonly in patients. The cell line also has a TP53 mutation, a homozygous deletion of CDKN2A/P16, and a moderate stromal phenotype. MIA PaCa-2 tumors were grown subcutaneously in twelve athymic nu/nu mice to a target size of 125 mm³. Mice were then randomized (n=6) and then received either no treatment or the optimized ¹³¹I-ELP and CP-PTX combination therapy. A summary of experimental conditions can be found in Table 6. The results, shown in FIG. 27A (FIG. 28), demonstrated a 100% ORR with complete tumor regression in all treated mice. Median survival almost tripled from 33 days for untreated mice to 92 days for mice receiving ¹³¹I-ELP and CP-PTX combination therapy (FIG. 27B, p<0.05 Mantel Cox log-rank test). At 107 days post-treatment, two of the mice continued to remain in remission until completion of the study.

TABLE 6

| | Tumor | Body | | | | | Median | | |
|---|---|---|---|---|---|---|---|---|---|
| Treatment | Size | Wt | | ¹³¹I-ELP | CP-PTX | CP-PTX | Survival | % | % |
| Group | (mm³) | (g) | n | Bose | (mg/kg) | Frequency | (d) | PR | CR |
| High Dose ¹³¹I-ELP | 107.6 ± 17.2 | 27.7 ± 2.1 | 7 | 10.6 uCi/mg | 12.5 | 4×, q.w. | 99.0 | 71.4 | 28.6 |
| Combotherapy | | | | | | | | | |
| Med. Dose ¹³¹I-ELP | 128.9 ± 36.4 | 27.7 ± 2.3 | 6 | 6.41 uCi/mg | 12.5 | 4×, q.w. | 90.5 | 83.3 | 0.0 |
| Combotherapy | | | | | | | | | |
| Untreated Tumors | 101.5 ± 30.2 | 27.9 ± 1.6 | 7 | n/a | n/a | n/a | 45.0 | 0.0 | 0.0 |

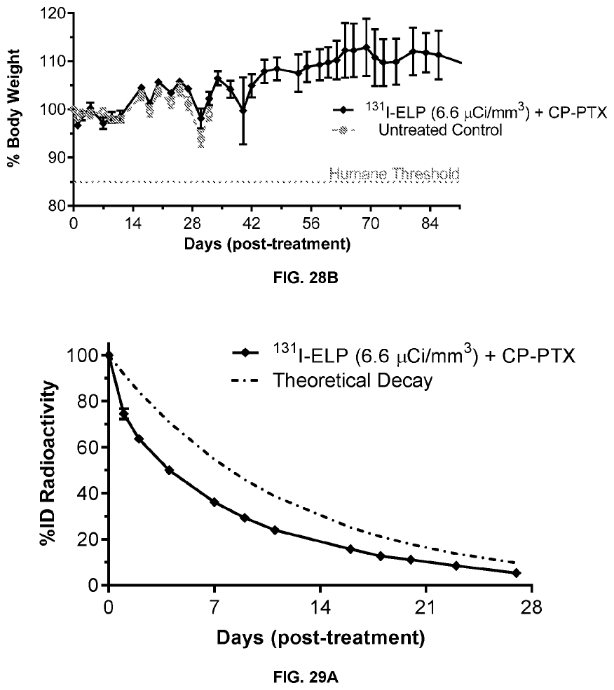

Body weight was also tracked as a surrogate measure of acute toxicity Table 6. No body weight loss was observed in any group and treatment mice were statistically insignificant compared to untreated animals (FIG. 29A, p=0.5663, 2-way ANOVA) and the radiodepot stability profile looked comparable to previous work (FIG. 29B).

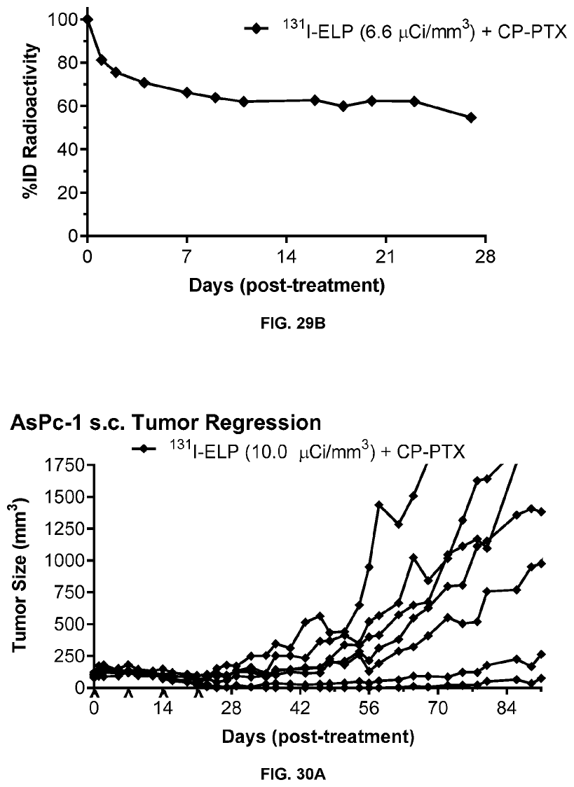

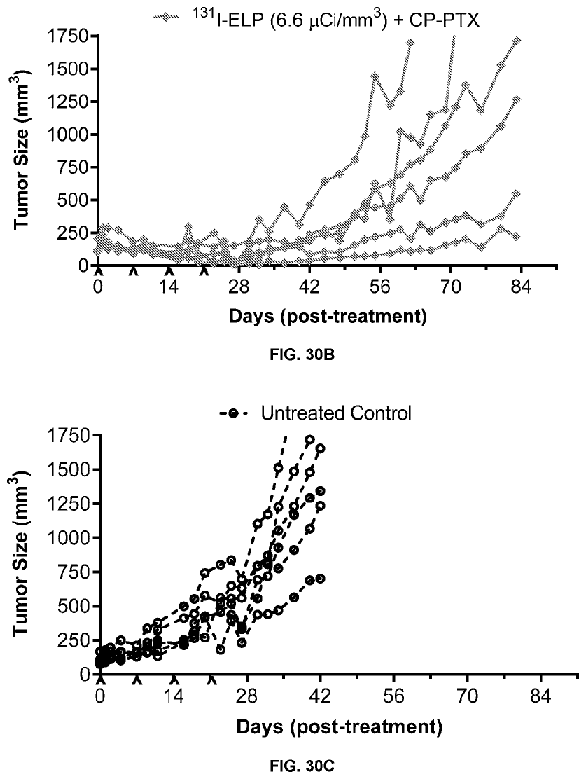

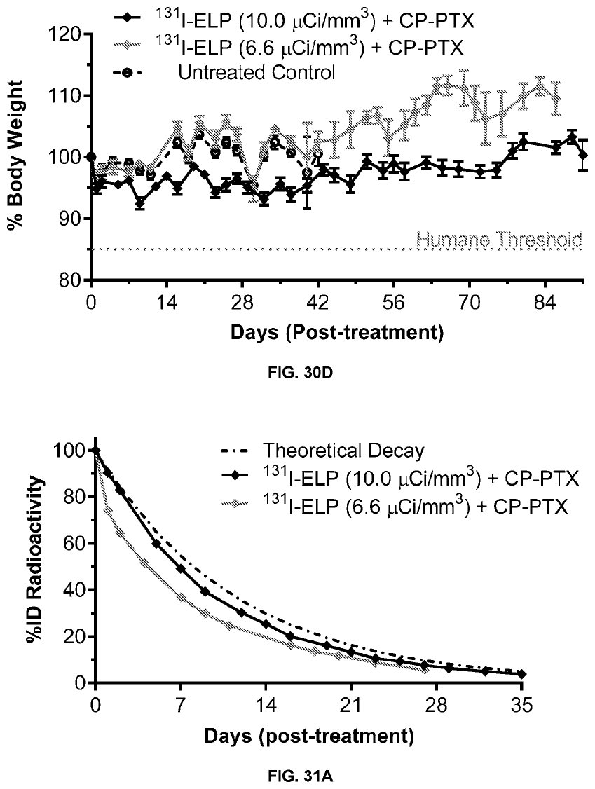

The anti-tumor efficacy in an AsPc-1 tumor xenograft model was examined next. It has genetic mutations in K-ras and TP5353, as well as deletions of CDKN2A/P16, SMAD4, MAP2K4, and FBXW7. Unlike the phenotype of MIA PaCa-2, AsPc-1 tumors are hypovascular and have high stromal density. AsPc-1 cells were inoculated subcutaneously in 14 athymic nu/nu mice and grown to the same target size of 125 mm³ (FIG. 30). The mice were divided into two groups (n=7) and either remained untreated or received the optimized combination therapy of ¹³¹I-ELP at 10 μCi/mm³ and 12.5 mg/kg of i.v. CP-PTX, once weekly. A summary of experimental conditions can be found in Table 7.

TABLE 7

| | Tumor | Body | | | | | Median | | |
|---|---|---|---|---|---|---|---|---|---|
| Treatment | Size | Wt | | ¹³¹I-ELP | | CP-PTX | Survival | % | % |
| Group | (mm³) | (g) | n | Dose | CP-PTX | Frequency | (d) | PR | CR |
| High Dose ¹³¹I-ELP | 107.6 ± 17.2 | 27.7 ±2 .1 | 7 | 10.6 uCi/mg | 12.5 mg/kg | 4×, q.w.. | 99.0 | 71.4 | 28.6 |
| Combotherapy | | | | | | | | | |
| Med. Dose ¹³¹I-ELP | 128.9 ± 36.4 | 27.7 ± 2.3 | 6 | 6.41 uCi/mg | 12.5 mg/kg | 4×, q.w. | 90.5 | 83.3 | 0.0 |
| Combotherapy | | | | | | | | | |
| Untreated Tumors | 101.5 ± 30.2 | 27.9 ± 1.6 | 7 | n/a | n/a | n/a | 45.0 | 0.0 | 0.0 |

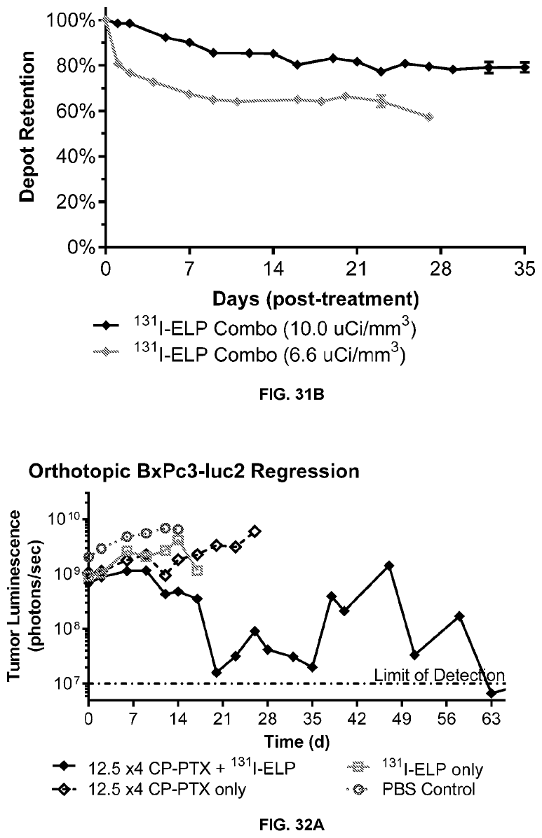

As seen in FIG. 27C, combination therapy resulted in highly significant tumor regression over untreated tumors (p<0.001, repeated measures ANOVA). The combination of ¹³¹I-ELP and CP-PTX induced a 100% ORR tumor response in the AsPc-1 model, however only 2 of 7 achieved a complete response. No body weight loss was observed in either the treated or untreated animals. The stability of the higher dose depot was seen to be significantly better, but the retention rates of both groups were deemed within acceptable parameters for experimental evaluation (FIG. 31). Median survival (FIG. 27D) was extended from 45 days for untreated mice to 99 days with combination therapy (p<0.05, Mantel Cox log-rank test).

Finally, the combination of ¹³¹I-ELP with systemic CP-PTX in an orthotopic BxPc3-luc2 tumor model was assessed. While lacking the prototypical K-ras mutation, BxPc3 is highly resistant due to constitutive mutations of TP53 and SMAD4 and further deletions of the CDKN2A/P16 and MAP2K4 genes. In addition, orthotopic tumor models have proven to be notoriously more resistant to treatment than subcutaneous xenografts due to their anatomical location and higher stromal content. In histochemical examination of subcutaneous and orthotopic BxPc3 tumors (see Example 12), Masson Trichrome staining clearly revealed the higher stromal collagen content of the orthotopic grafts. As BxPc3-luc2 was stably transfected with the firefly luciferase gene, it enabled non-invasive monitoring of tumor responses to treatment using the IVIS Lumina XR by monitoring changes in tumor luminescent flux41 (FIG. 32). Tumors were surgically implanted in the pancreas and grown for 21 days, reaching a size of 169.5 mm³. A summary of experimental conditions can be found in Table 8.

TABLE 8

| | Tumor | Body | | | | | Median | | |
|---|---|---|---|---|---|---|---|---|---|
| Treatment | Size | Wt | | ¹³¹I-ELP | | CP-PTX | Survival | % | % |
| Group | (mm³) | (g) | n | Dose | CP-PTX | Frequency | (d) | PR | CR |
| ¹³¹I-ELP Combotherapy (12.5 × 4) | 169.5 | 30.3 ± 2.2 | 6 | 8.83 uCi/mg | 12.5 mg/kg | 4×, q.w. | 58.0 | 16.7 | 83.3 |
| 12.5 × 4 CP-PTX only | 167.9 | 31.4 ± 1.6 | 6 | n/a | 12.5 mg/kg | 4×, q.w. | 26.0 | 16.7 | 0.0 |
| ¹³¹I-ELP Combotherapy (25 × 2) | 175.0 | 29.8 ± 1.8 | 6 | 7.90 uCi/mg | 25 mg/kg | 2×, q.w. | 63.0 | 16.7 | 83.3 |
| 25 × 2 CP-PTX only | 197.5 | 30.8 ± 2.7 | 6 | n/a | 25 mg/kg | 2×, q.w. | 14.0 | 16.7 | 0.0 |
| ¹³¹I-ELP only | 183.4 | 29.3 ± 2.6 | 6 | 7.06 uCi/mg | n/a | n/a | 15.5 | 16.7 | 16.7 |
| Untreated Tumors | 197.5 | 30.8 ± 2.3 | 6 | n/a | n/a | n/a | 15.5 | 0.0 | 0.0 |

At the time of treatment, the tumors were surgically accessed and injected with the 10 μCi/mm³ of ¹³¹I-ELP. CP-PTX was administered i.v. once weekly at a paclitaxel equivalent dose of 12.5 mg/kg for four weeks. Monotherapy groups (n=6) of CP-PTX and ¹³¹I-ELP treatments were also included. Monotherapies demonstrated minimal growth inhibition over untreated tumors specimens (FIG. 27E). However, ¹³¹I-ELP and CP-PTX combination therapy achieved a 100% ORR with 83.3% achieving a complete response where no luminescent tumor signal was detectable. Median survival for combination therapy treated mice was 58 d (p<0.01, Mantel-Cox log-rank test) as opposed to 15.5 d for untreated tumors (FIG. 27F).

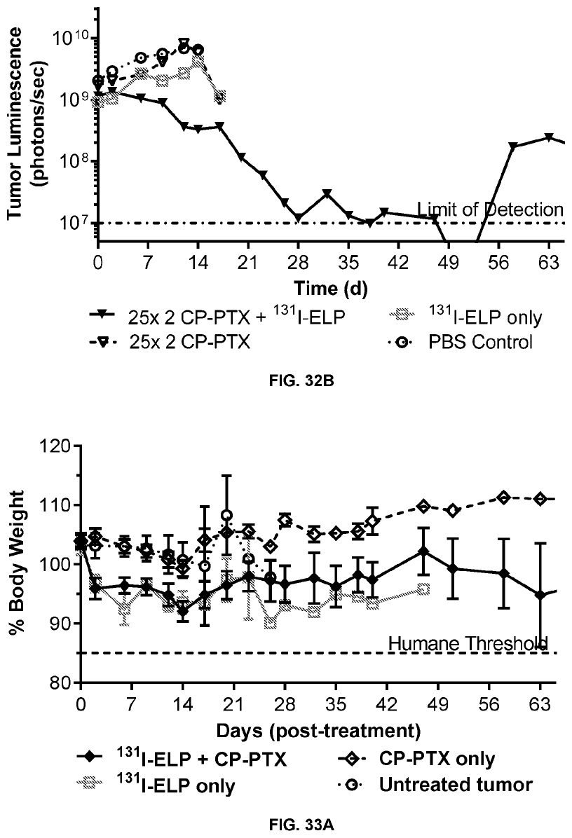

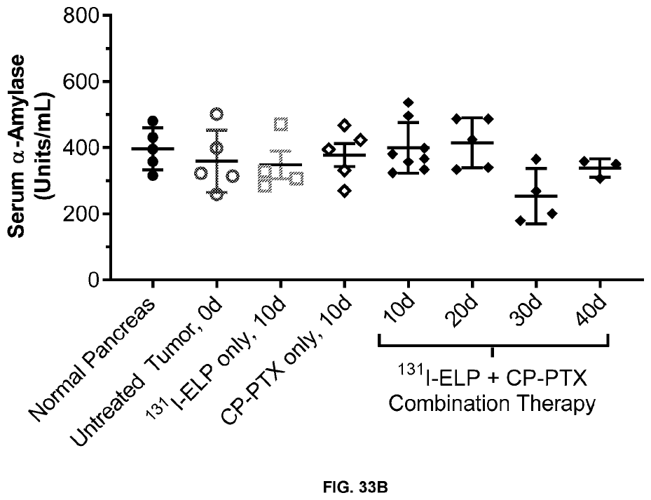

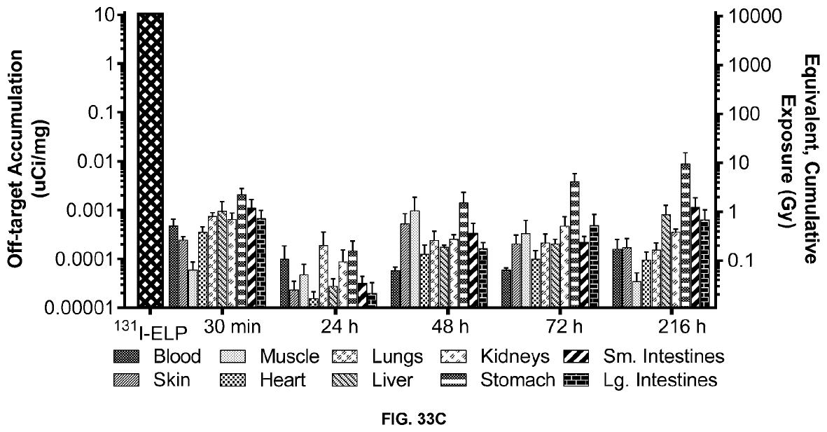

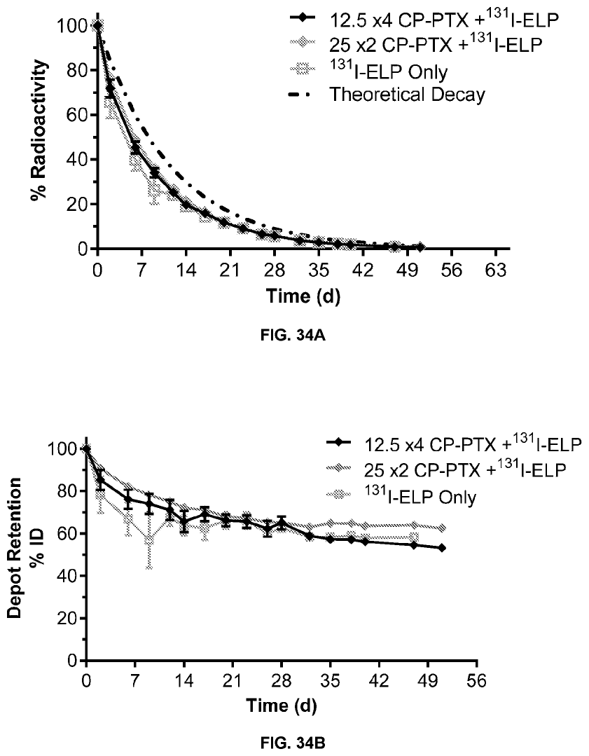

In addition to efficacy, the orthotopic BxPc3-luc2 model provided a more rigorous model for assessing possible toxic side effects of the ¹³¹I-ELP brachytherapy. No significant body weight loss was observed due to ¹³¹I-ELP monotherapy or combination treatment (FIG. 33A). A small loss of 7-9% was observed following completion of surgery, but this is typical of the procedure. Mice recovered the weight loss within a day. Blood samples were collected from the treatment groups at various time points and assayed for circulating levels of the pancreatic damage marker, α-amylase. These results (FIG. 33B) showed that all treatment groups compared favorably to untreated mice, as well as healthy mice. Serological levels remained equivalent to healthy mice regardless of treatment group or the duration of ¹³¹I-ELP combination therapy. This was not completely unexpected as non-cancerous pancreatic tissue in the TUNEL IHC specimens of FIG. 19 showed minimal TUNEL staining in contrast to the high levels of apoptosis evident in the tumor tissue. A serial biodistribution study was next conducted on an additional group of orthotopically treated mice to determine accumulation of ¹³¹Iodine in healthy tissues. Groups of mice (n=4) were euthanized at time points of 30 min, 24 h, 48 h, 72 h, and 216 h following intratumoral injection of ¹³¹I-ELP and then dissected. The radiation activity of vital tissues was assayed and a total exposure over ten decay half-lives was calculated (FIG. 33C). Except for the stomach, all tissues exhibited off-target accumulation levels under 0.001 μCi/mg. This equates to a cumulative exposure of less than 1 Gy. Radioactivity levels in the stomach ranged from 0.0005 μCi/mg up to 0.013 μCi/mg. It is unclear if this was due to true off-target accumulation, or the result of mice ingesting contamination from the cage bedding. As the radioactivity increased with time, the latter is more likely and confounds analysis. The stability of the radiodepot was also observed (FIG. 34).

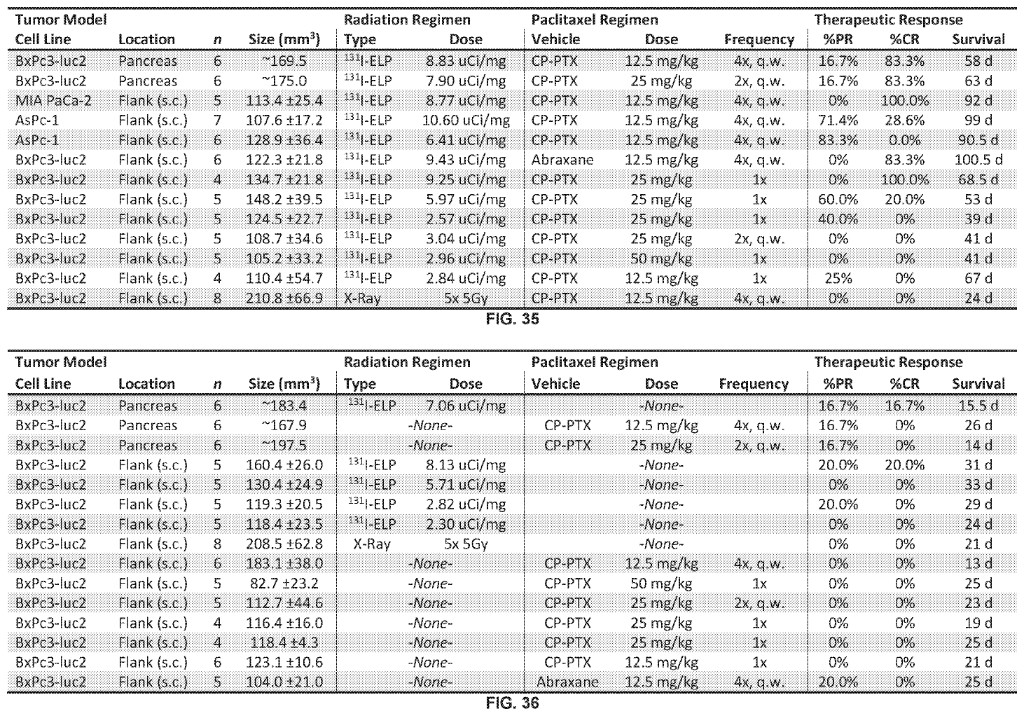

Summaries of the results from all the tumor regression studies are shown in FIG. 35 and FIG. 36.

### Example 8: Characterization of CP-PTX Micelles

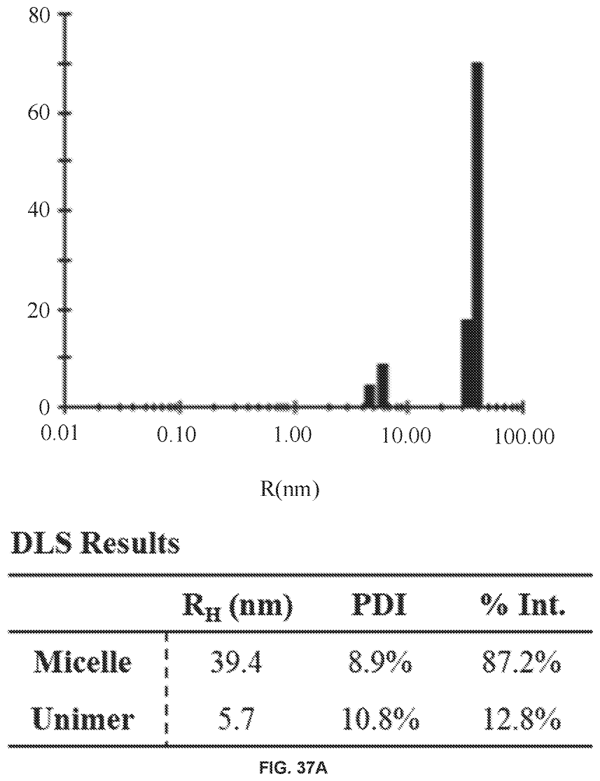

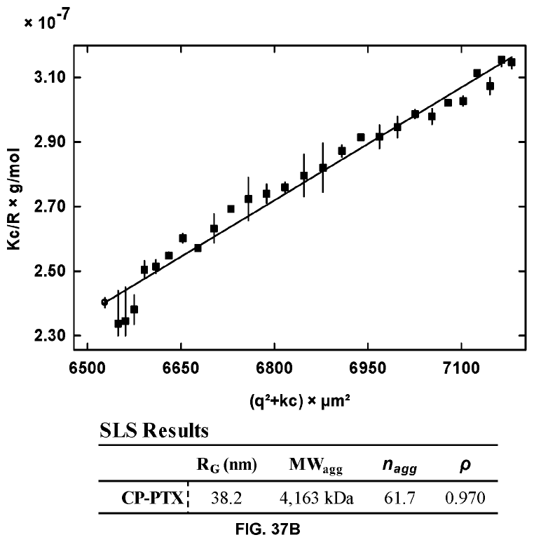

The CP-PTX conjugates were characterized by dynamic light scattering (DLS), as follows. FIG. 37A shows a predominant nanoparticle population with a R_{H} of 39.4 nm. A small unimer population (12.8%) was identified with a Rₕ of 5.7 nm, as expected for unimers. Both populations were highly monodisperse.

The same CP-PTX solution was then diluted to 3 mg/kg and analyzed to determine the radius of gyration (R_{G}) by static light scattering. Static light scattering was performed at 37° C. at 5° angle increments between 30° and 150°. Measurements at each angle consisted of an average of 3 different 15 s exposures with a dRate %<5%. The partial Zimm plot was analyzed to determine the R_{G} and the molecular weight (FIG. 37B). From this, the number of polymer chains in a micelle, n_{agg}, and the shape factor ρ was determined.

### Example 9. Bliss Independence—Model Explanation & Mathematics

The Bliss Independence model is based on measuring the fraction of effect of each single agent and probabilistically determining their combined effect. For tumor regression, this involves first measuring the respective tumor volume after treatment with a single agent and comparing it to an untreated tumor control to calculate

$${f}_{i} = \frac{{Vol}_{i}}{V o {l}_{Untreated}} ;$$

where i corresponds to the therapeutic agent. The predicted response for the combinatorial treatment, if all agents act independently of each other, is then formulated as the product of their respective fraction of effects.

$${f}_{Bliss Predicted} = {\prod }_{i = 1}^{n} {f}_{i} , for n agents$$

For a dual agent combination therapy regimen, this reduces to f_{Bliss Predicted}=f₁f₂.

Synergy was thus defined as a greater observed therapeutic response than predicted by the Bliss probabilistic function, while agent antagonism was defined as a reduced response. For tumor regression studies, this meant that the fraction of tumor remaining after dual treatment would be much less than that predicted by the model. Mathematically, this was represented as:

$${f}_{Observed} \{ \begin{matrix}> {f}_{Predicted} , Bliss Antagonism \\ = {f}_{Predicted} , Independent Mechanisms \\ < {f}_{Predicted} , Bliss Synergy\end{matrix} .$$

By utilizing the variance and mean of the predicted Bliss value, traditional parametric analysis was applied to qualify the statistical significance of the synergy associated with the observed effects.

Var(f_{Bliss})=Var(f₁f₂)

This was expanded using the variance identity for the product of two independent variables.

Var(f_{Bliss})=E(f₁)²Var(f₂)+E(f₂)²Var(f₁)+Var(f₁)Var(f₂)

E(X) is the expected value (average) for a number set X with m elements.

E(X)=Σⱼ₌₁^{m}Xⱼ/m

This identity was further simplified to yield the formulae used to assess the Variance and Standard Deviation of the predicted Bliss Independent value.

Var(f_{Bliss})=E(f₁²)E(f₂²)−E(f₁)²E(f₂₎²

σ_{Bliss Predicted}=\sqrt{E(f₁²)E(f₂²)−E(f₁)²E(f₂)²},

### Example 10: Effective Radiation Half-Life & Depot Biological Half-Life

The effective radiation half-life of the ¹³¹I-ELP brachytherapy depots was experimentally determined by measuring the whole-body radioactivity of treated mice as a function of time. When combined with tissue biodistribution results, this showed that the tumor depot accounted for >99% of the radioactivity signal; therefore, longitudinal whole-body monitoring was deemed an acceptable approximate substitute for depot activity.

Activity at a given time point was normalized against the initial activity to determine the percent injected dose (% ID). This decay profile was then log transformed to fit the following linear equation:

$$ln ( \% ID ) = ln ( \frac{A}{{A}_{o}} ) = ( - \frac{ln ( 2 )}{{t}_{1 / 2}} ) t .$$

A simple linear regression was done to determine the slope, b which was used to calculate the effective half-life according to the relationship:

$${t}_{1 / 2} = \frac{- ln ( 2 )}{b} .$$

The depot biological half-life was determined following a similar procedure, but after accounting for the physical decay of the isotope (t_{1/2}=8.03 days). This linear equation is:

$$ln ( \frac{\% ID}{{e}^{- l n ( 2 ) t / 8.03}} ) = ( - \frac{ln ( 2 )}{{t}_{1 / 2}} ) t .$$

Results were tabulated for each individual mouse and averaged within an experimental group.

TABLE 9

| | Tumor | | | ¹³¹I-ELP | PTX | Effective | Depot |
|---|---|---|---|---|---|---|---|
| Experiment | Line | Model | n | Dose | Dose | t_{1/2} (d) | t_{1/2} (d) |
| Radiodose Escalation | BxPc3-luc2 | Subcut. | 4 | 9.25 uCi/mg | 25 mg/kg | 6.140 | 26.510 |
| Trial | | | | | | | |
| Radiodose Escalation | BxPc3-luc2 | Subcut. | 5 | 8.13 uCi/mg | 0 mg/kg | 6.381 | 31.545 |
| Trial | | | | | | | |
| Radiodose Escalation | BxPc3-luc2 | Subcut. | 5 | 5.97 uCi/mg | 25 mg/kg | 6.579 | 36.963 |
| Trial | | | | | | | |
| Radiodose Escalation | BxPc3-luc2 | Subcut. | 5 | 5.71 uci/mg | 0 mg/kg | 6.633 | 38.329 |
| Trial | | | | | | | |
| Radiodose Escalation | BxPc3-luc2 | Subcut. | 5 | 2.57 uCi/mg | 25 mg/kg | 6.822 | 49.287 |
| Trial | | | | | | | |
| Radiodose Escalation | BxPc3-luc2 | Subcut. | 5 | 2.30 uCi/mg | 0 mg/kg | 6.840 | 50.919 |
| Trial | | | | | | | |
| Paclitaxel Dose | BxPc3-luc2 | Subcut. | 5 | 2.96 uCi/mg | 50 mg/kg | 7.133 | 66.781 |
| Escalation Trial | | | | | | | |
| Paclitaxel Dose | BxPc3-luc2 | Subcut. | 5 | 2.79 uCi/mg | 25 mg/kg | 7.348 | 91.882 |
| Escalation Trial | | | | | | | |
| Paclitaxel Dose | BxPc3-luc2 | Subcut. | 4 | 2.84 uCi/mg | 12.5 mg/kg | 6.754 | 44.847 |
| Escalation Trial | | | | | | | |
| Paclitaxel Dose | BxPc3-luc2 | Subcut. | 5 | 3.04 uCi/mg | 25 mg/kg | 7.287 | 82.334 |
| Escalation Trial | | | | | | | |
| Paclitaxel Dose | BxPc3-luc2 | Subcut. | 5 | 2.82 uCi/mg | 0 mg/kg | 7.158 | 70.687 |
| Escalation Trial | | | | | | | |
| Abraxane Comparison | BxPc3-luc2 | Subcut. | 6 | 9.43 uCi/mg | 12.5 mg/kg | 6.965 | 53.222 |
| Trial | | | | | | | |
| MIA PaCa-2 Tumor | MIA PaCa-2 | Subcut. | 5 | 8.77 uCi/mg | 12.5 mg/kg | 6.849 | 48.815 |
| Trial | | | | | | | |
| AsPc-1 Tumor | AsPc-1 | Subcut. | 7 | 10.6 uCi/mg | 12.5 mg/kg | 7.408 | 99.887 |
| Trial | | | | | | | |
| Orthotopic Efficacy | BxPc3-luc2 | Orthotopic | 6 | 8.83 uCi/mg | 12.5 mg/kg | 6.824 | 56.925 |
| Trial | | | | | | | |
| Orthotopic Efficacy | BxPc3-luc2 | Orthotopic | 6 | 7.90 uCi/mg | 25 mg/kg | 7.104 | 69.631 |
| Trial | | | | | | | |
| Orthotopic Efficacy | BxPc3-luc2 | Orthotopic | 6 | 7.06 uCi/mg | 0 mg/kg | 6.403 | 30.390 |
| Trial | | | | | | | |

### Example 11. Dose Rate & Cumulative Dose Calculation

TABLE 10
Therapeutic Values, Physical Quantities and Unit Conversions

| Symbol | Meaning | Value | | Standard Units | |
|---|---|---|---|---|---|
| Aₒ | ¹³¹I-ELP Injection Dose | 10 | μCi/mg | 10 | Ci/kg |
| V_{Tumor} | Average Tumor Volume | 125 | mg | 1.25 × 10⁻⁴ kg | |
| V_{Depot} | Injection volume of ELP depot | ⅓V_{Tumor} | | ⅓V_{Tumor} | |
| t_{1/2, physical} | Physical half-life of isotope | 8.03 | days | 8.03 | days |
| t_{1/2, eff} | Effective half-life of radioactive depot | 6.880 | days | 6.880 | days |

| k_{Ci} | Conversion factor from Curie | 3.7 × 10¹⁰ | | 3.197 × 10¹⁵ |
|---|---|---|---|---|
| | to decay events | events/sec | | events/day |
| k_{keV} | Conversion factor from keV to Joules | 1.60218 × 10⁻¹⁶ J | | 1.60218 × 10⁻¹⁶ J |
| E_{β, avg} | Average energy of beta particle emissions | 181.86 | keV | 2.914 × 10⁻¹⁴ J |
| E_{γ, avg} | Average energy of gamma particle emissions | 381.97 | keV | 6.120 × 10⁻¹⁴ J |

| | |
|---|---|
| Eᵢ | Energy of a single emissions particle (i) from a decay event |
| Iᵢ | Intensity, or statistical frequency, of a particle emission from a decay event |
| f abs | Fraction of absorption of emission particle within the tumor geometry |

### Assumptions

1. Total dose was estimated assuming a homogenous dose distribution
2. Emission spectra, energies, and intensity values obtained from the National Nuclear Data Center, Brookhaven National Laboratory was accurate.
3. The range of tissue penetration (l_{β}) for 131Iodine β-particles ≤0.8 mm. Average=0.4 mm.
4. The volume of the intratumoral depot V_{Depot} was approximately equal to the injected ELP volume.
5. Dose_{β}>>Dose_{γ}, therefore Total Dose≈Dose_{β}
6. 98.6% of β-particles were absorbed within the tumor margin (f_{abs}=0.9859)

### Quick Calculation of Depot Coverage in Spherical Tumor Model

$${V}_{Depot} = 1 / 3 {V}_{Tumor}$$

$$4 / 3 π {r}_{Depot}^{3} = 1 / 3 {V}_{Tumor}$$

$${r}_{Depot} = \sqrt[3]{\frac{{V}_{Tumor}}{4 π}}$$

For a 125 mm³ tumor:
r_{Tumor}=3.10 mm and r_{Depot}=2.15 mm

The activity (A) of ¹³¹Iodine can be determined at any time point t using the exponential decay equation: A=Aₒe^{-rt} where r=ln(2)/t_{1/2eff}.

The rate of radioactive decay events was defined as shown below.

$${Rate}_{Decay Events} = A \times {k}_{Ci}$$

$${Rate}_{Decay Event} = {A}_{o} {k}_{Ci} {e}^{- rt}$$

The total number of decay events were determined by integrating with respect to time.

$$Total events = {\int }_{0}^{t} {A}_{o} {k}_{Ci} {e}^{- rt} dt$$

$$Total events = \frac{{A}_{o} {k}_{Ci}}{r} ( {e}^{rt} - 1 )$$

$$Total events = \frac{{A}_{o} {k}_{Ci} {t}_{\frac{1}{2} eff}}{ln ( 2 )} ( 1 - {e}^{- ln ( 2 ) * t / {t}_{\frac{1}{2} eff}} )$$

To determine the total dose, the total number of each emission particle was determined based on the number of decay events. The energy of each particle was multiplied by the function describing the particle's absorption within the tumor margins, which was approximated to be 98.6% for β-particles.

$${Dose}_{Total} = {Dose}_{β} + {Dose}_{γ}$$

$${Dose}_{Total} \tilde{=} {Dose}_{β}$$

$${Dose}_{β} = Total events * {k}_{ke V} {\sum }_{i = 1}^{n} {I}_{i} {E}_{i} {f}_{abs , i}$$

where I={1 . . . n} is the set of all β-particle emissions of ¹³¹Iodine

$${Dose}_{β} = \frac{{A}_{o} {k}_{Ci} {k}_{k e V} {t}_{\frac{1}{2} eff}}{ln ( 2 )} ( 1 - {e}^{- ln ( 2 ) * t / {t}_{\frac{1}{2} eff}} ) * {\sum }_{i = 1}^{n} {I}_{i} {E}_{i} {f}_{abs , i}$$

Simplifying for the case where t→∞ and the assumption f_{abs,i}→1, then E_{β,avg}=Σᵢ₌₁ⁿIᵢEᵢ.

$${Dose}_{Total} = {Dose}_{β} = \frac{{A}_{o} * {k}_{C i} * {k}_{k e V} * {t}_{\frac{1}{2} eff} * {f}_{abs} * {E}_{β , avg}^{ }}{ln ( 2 )} \qquad$$

### Final Result: Dose_{Total}(@10 μCi/mg)=9145 Gray

### Example 12: Pathological Analysis of BxPc3-Luc2 Tumor Specimens after Comparative Treatments

In order to elucidate the mechanistic underpinnings of the synergy observed between ¹³¹I-ELP and paclitaxel, a study was carried out whereby treated tumor specimens were histologically examined for changes in the tumor microenvironment. BxPc3-luc2 tumors were grown orthotopically in athymic, nu/nu mice. Upon reaching a size of ˜100 mm³, mice were sorted into groups of untreated tumors (n=3), CP-PTX monotherapy (n=2), external beam radiation only (n=4), ¹³¹I-ELP only (n=3), EBRT with CP-PTX combination therapy (n=5), and ¹³¹I-ELP with CP-PTX combination therapy (n=4). CP-PTX was given as two, weekly i.v. injections at 12.5 mg/kg of paclitaxel equivalent for all relevant groups. EBRT was administered as five 5 Gy X-ray fractions every other day for a total of 25 Gy. ¹³¹I-ELP was administered as 10.0 μCi/mm³. 12 after treatment, animals were euthanized, and tumors were excised for histology processing. This duration ensured tumors treated with EBRT would receive all of their X-ray fractions.

Additionally, tumors treated with ¹³¹I-ELP brachytherapy and CP-PTX chemotherapy were observed to begin regressing at 14 days. Therefore, 12 days would allow for maximal change to the microenvironment without regression complicating the analysis. Two normal pancreas samples were also collected from healthy mice for reference.

The tumor specimens were excised and stored in formalin. The specimens were stored for 8 months to allow for complete decay of the 131I for safe histological handling. Tumors were then paraffin embedded, sectioned from the center of the tumor outwards, and mounted as 7 μm slices. The specimens were then stained with a variety of immunohistochemical stains to examine various features of the tumor microenvironment: H&E, Masson Trichrome, CD-31 (ThermoFisher, #PA5-16301), CD-144 (ThermoFisher, #36-1900), anti-Claudin-4 (ThermoFisher, #PA5-16875), and TUNEL (Millipore, #S7100).

All specimens were then randomized and blinded with reference to their treatment group in order to prevent bias in the pathological interpretation. Blinded samples were then analyzed. Specimens were commented on and scored on the intensity of each respective stain, as well as the frequency of cellular expression.

Together, the scores were combined to create an H-score. The relative coverage of a marker, or % area of positive staining, was created for IHC stains by spectrally isolating the horseradish peroxidase stain, converting it to a binary mask, and then quantifying its area compared to the area of the whole tumor specimen using ImageJ. Once analyzed, the data were unblinded, and the results were aggregated to yield the following observations in Supp. Section L.1-L.6.

H&E Analysis

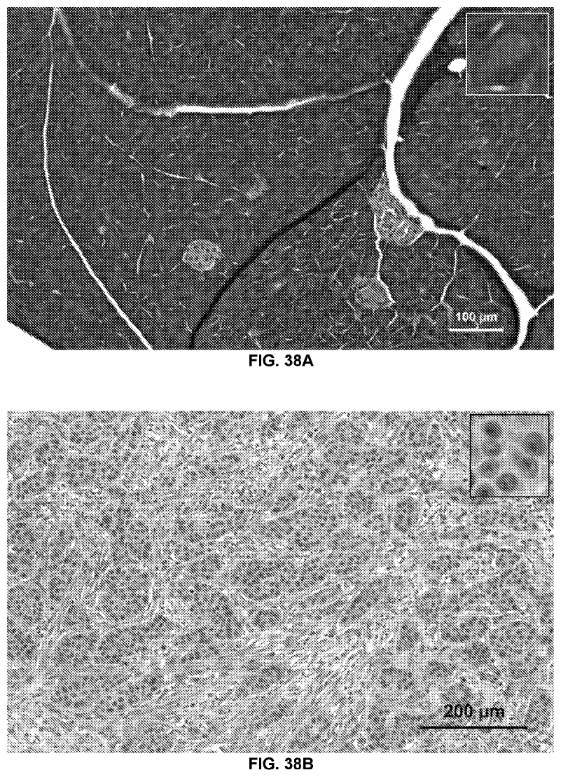

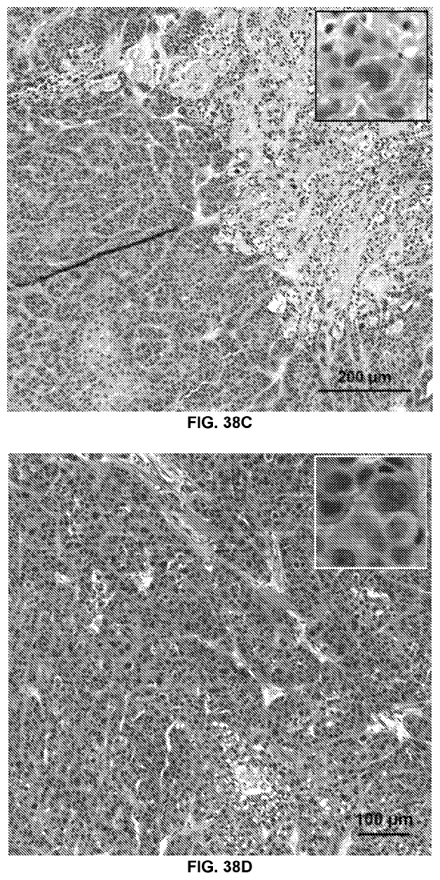

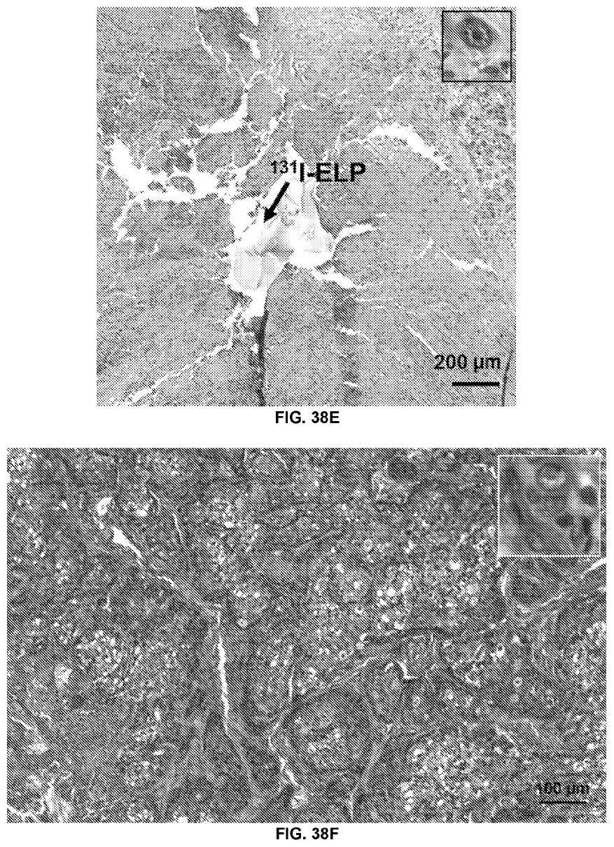

The pancreas from healthy mice showed normal pancreatic acini cells with occasional islets of Langerhans. The BxPc3-luc2 tumors, meanwhile, were clearly comprised of dense adenocarcinoma cells with a desmoplastic, fibrous stroma (FIG. 38). A small percentage of these tumor specimens showed evidence of necrosis (1.67%) even without any treatment. Typically, the area of these foci was small, with radii of ˜0.6 mm.

The effects of each monotherapy were first assessed prior to analyzing the combination therapy results. Tumors treated with CP-PTX showed the same distinctive adenocarcinoma cell pathology. However, the percentage of the tumor specimen demonstrating necrosis was larger at 33%. This necrosis was non-uniform across the tumor tissue, presenting as distinct patches while the bulk of the tumor tissue remained unaffected. When the tumor samples treated with X-ray EBRT only, the adenocarcinoma tissue remained abundant in desmoplastic stroma. A slight increase in cellular necrosis was apparent over the untreated tumor, but only accounted for 9% of the total tumor. For the tumors treated with ¹³¹I-ELP, however, different features were observed. Two-thirds of treated specimens showed clear evidence of pleomorphism, particularly in proximity to the depot material. Scars of necrosis accounted for 30% of the total tumor area and were also located radially around the depot material. Finally, evidence of pyknotic cells was also witnessed.

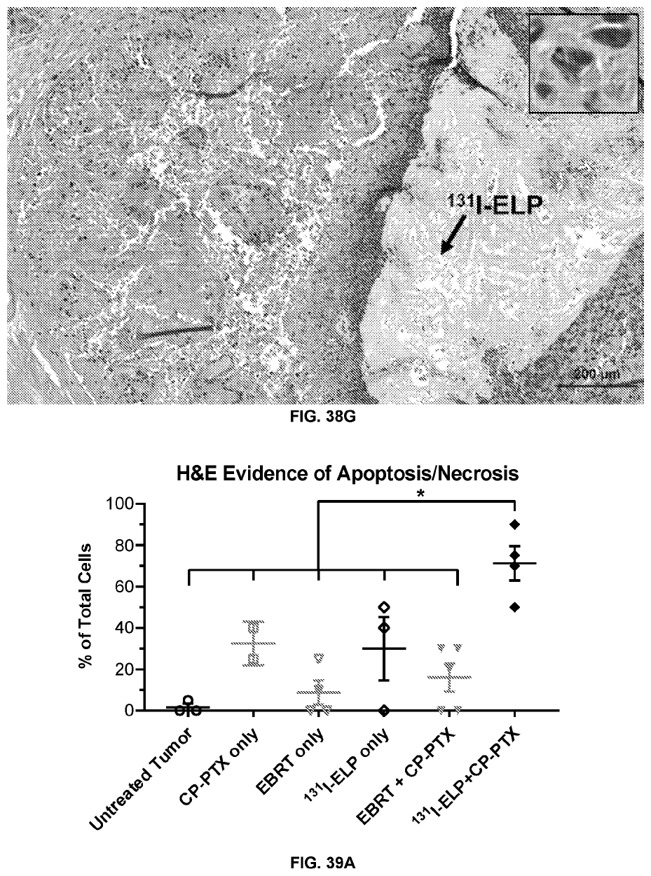

Next, the effects of combination therapy of CP-PTX chemotherapy with either X-ray EBRT or ¹³¹I-ELP brachytherapy were compared (FIG. 39). Tumors treated with EBRT combination therapy exhibited typical adenocarcinoma traits, but with centralized fibrosis and patchy foci of necrosis. This necrosis only accounted for ˜16% of the total tumor specimen. A small amount of pleomorphisms was identified in 2/5 specimens. ¹³¹I-ELP combination therapy, meanwhile, displayed large centralized areas of necrosis that accounted for 70% of the specimens on average. The adenocarcinoma was poorly differentiated with a high degree of pleomorphism exhibited in all samples. Interestingly, the necrosis and pleomorphism was always associated proximally with the depot, which could be visualized in some instances.

TUNEL (Apoptotic DNA Damage) Analysis

Terminal deoxynucleotidyl transferase dUTP nick end labeling, TUNEL, is a method whereby the 3′ hydroxyl termini of broken DNA strands are enzymatically tagged with TdT and then stained for histological analysis. Because it selectively identifies DNA strand breaks, it is a traditional IHC marker for apoptotic damage in tissues. TUNEL immunohistology was performed to visualize and spatially quantify the extent of apoptosis, as it would provide insight into the spatial limitations of ¹³¹I-ELP brachytherapy emissions are localized within a few millimeters of the injected biopolymer.

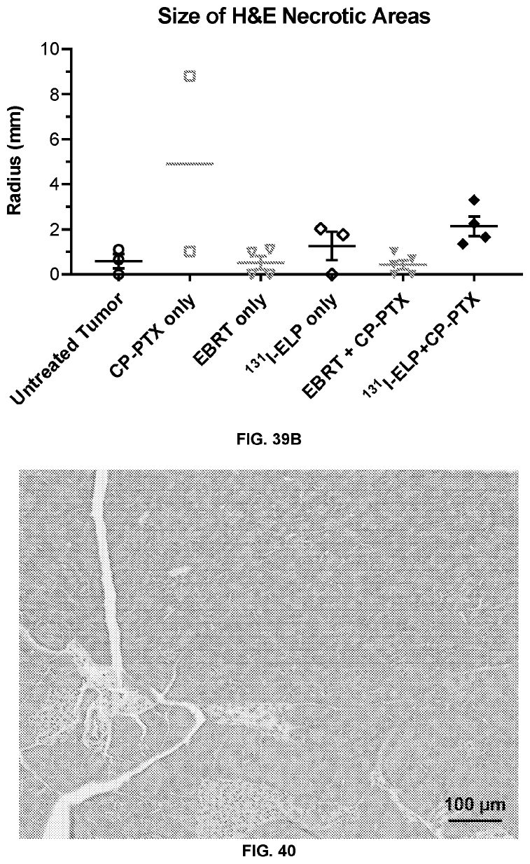

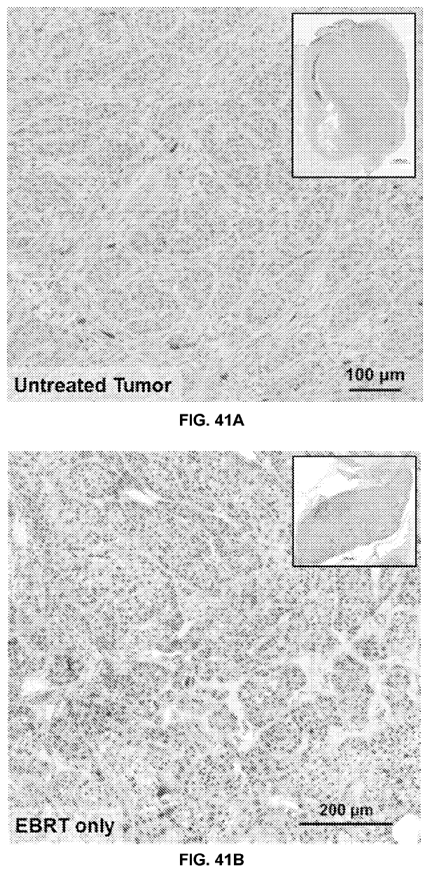

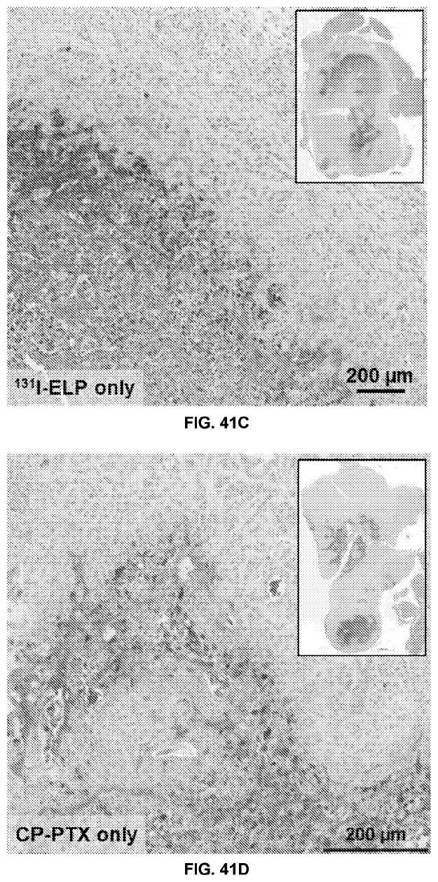

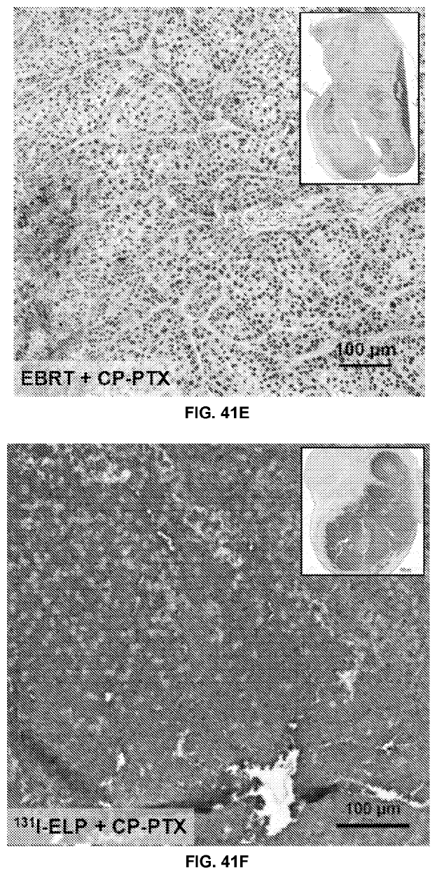

Healthy murine pancreatic tissue showed virtually no indication of DNA damage in either the acini cells or in the Langerhans islets, as expected (FIG. 40). The untreated BxPc3-lc2 tumor tissue was also mostly negative, although some light non-specific TUNEL staining was evident. The staining patterns amongst the different treatment groups, however, were very different. For tumors treated only with systemic CP-PTX, positive TUNEL staining appeared in patchy areas that were spatially distinct. Within those areas, staining was very strong. Outside those areas, which comprised the majority of the tumor, staining was mostly negative. Tumors treated with X-ray EBRT showed diffuse nuclear positivity across all regions of the tumor tissue. The staining was homogenous, although not as intense as the regions stained by CP-PTX or ¹³¹I-ELP monotherapies. The ¹³¹I-ELP brachytherapy only treatment showed extremely dense areas of necrosis. Despite the strength of this staining, the intensity fell off quickly, leaving distal areas with minimal staining. These results clearly show the focal nature of the ¹³¹Iodine emissions (FIG. 41).

The combination therapy regimens proved even more interesting. The tumors treated with EBRT and concomitant CP-PTX chemotherapy displayed a positive TUNEL staining pattern that looked distinctively like a merging of the two respective monotherapies. Diffuse nuclear positivity was evident throughout the entire bulk of the tumor cells. However, stronger staining patches also were evident throughout the tumor, as if combining therapies provided additive apoptotic consequences. The tumor samples from ¹³¹I-ELP combination therapy proved a starkly different. In all samples, the majority of the tumor was stained with an intense, dark carpet of TUNEL positivity. Very few viable cells could be identified due to the intensity of the stain. The staining was qualitatively stronger than any staining achieved by the monotherapies, showing the dramatic increase in apoptotic potency of this strategy. Even more interesting, the size of the apoptotic region was greatly increased from the ¹³¹I-ELP monotherapy. This suggested that the strategy of combining paclitaxel with ¹³¹I-ELP brachytherapy could actually extend the effective distance of ¹³¹I-ELP, which could improve radiation dosimetry planning in a clinical setting.

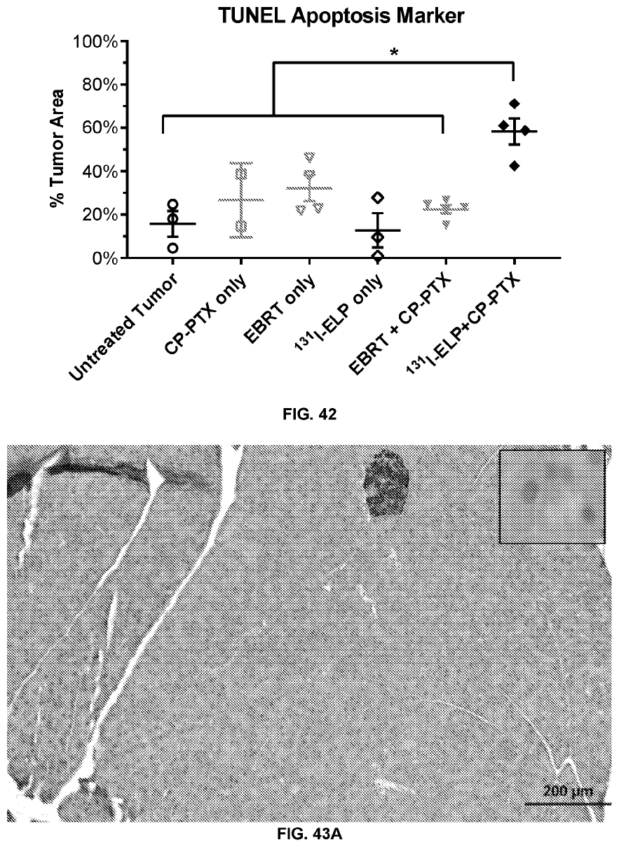

Finally, TUNEL staining provided an interesting way to observe the microscopic potency of the treatment regimens. Each specimen was spectrally deconvoluted using ImageJ to separate the TUNEL stain from the methyl green counterstain. It was then converted into a binary mask and the relative area of the two stains was compared to evaluate the area of apoptosis caused by the treatments. While this technique did not account for staining intensity, it did provide a method for analyzing the extent of apoptosis induced by each treatment method. ¹³¹I-ELP combination therapy induced significantly larger areas of tumor death compared to all other treatments (FIG. 42). The breadth of this coverage, combined with the intensity differences, further confirmed the drastic advantage provided by this strategy to treat pancreatic tumors.

Claudin-4 Analysis

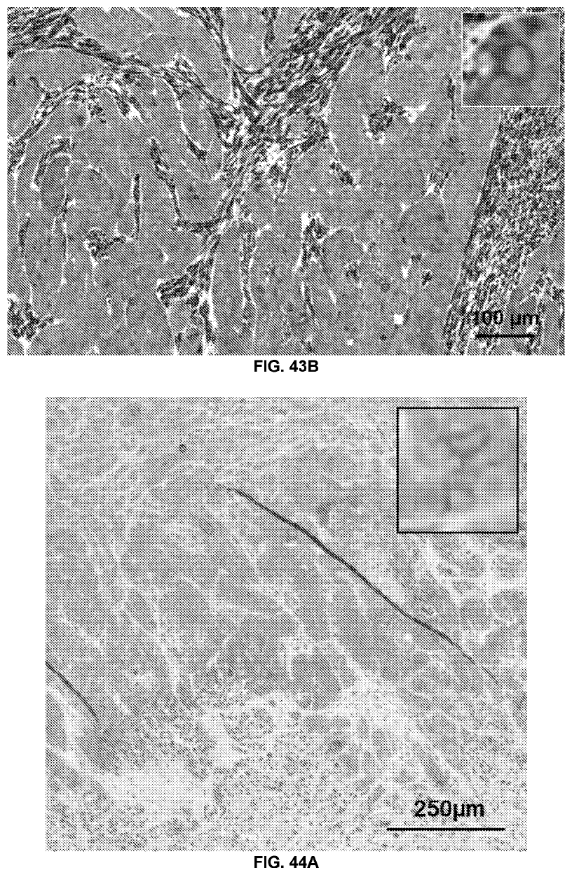

Claudin-4 expression was analyzed using a rabbit IgG with polyclonal Claudin-4 specificity, stained with horseradish peroxidase, and counterstained with nuclear methyl green. Claudin-4 is a transmembrane epithelial tight junction that forms a paracellular barrier for controlling molecular trafficking. It has been shown to be highly upregulated in human pancreatic tumors and is correlated with poor drug uptake and general resistance to chemotherapeutics (FIG. 43).

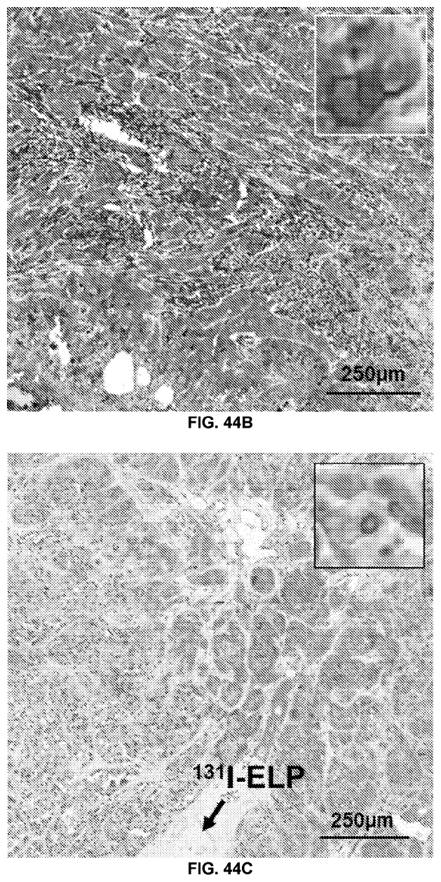

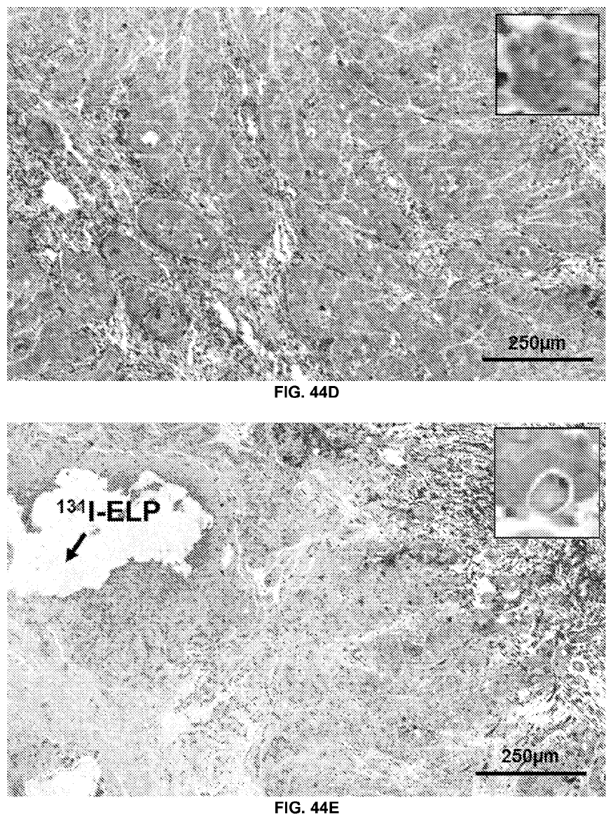

Normal murine pancreatic tissue was found to be negative for Claudin-4 expression in its acini cellular structure, as well as in the ductal epithelium. The Langerhans islets, however, demonstrated positive expression with strong staining. Untreated BxPc3-luc2 xenografts, conversely, displayed positive membranous and cell cytoplasm staining throughout the entire tumor tissue. The Claudin-4 intensity was also strong throughout the tumor stroma. Pathological analysis of the tumors subjected to different treatments showed no significant difference in the staining pattern amongst viable cells. The only small difference of note was the appearance of dot-like cytoplasmic staining pattern in the ¹³¹I-ELP combination therapy treatment specimens (FIG. 44).

It should be noted, though, that the areas proximal to the ¹³¹I-ELP depots groups could not be accurately read due to the high levels of apoptosis in the surrounding tissue. High levels of apoptosis can cause non-specific antibody staining due to degraded protein content that it is not necessarily representative of the actual microenvironment. Thus, the regions that corresponded to the intense TUNEL staining for ¹³¹I-ELP groups were inconclusive despite their irregularity of the staining pattern. This irregularity was found to diminish radially from the depots, at which point viable cells could be inspected.

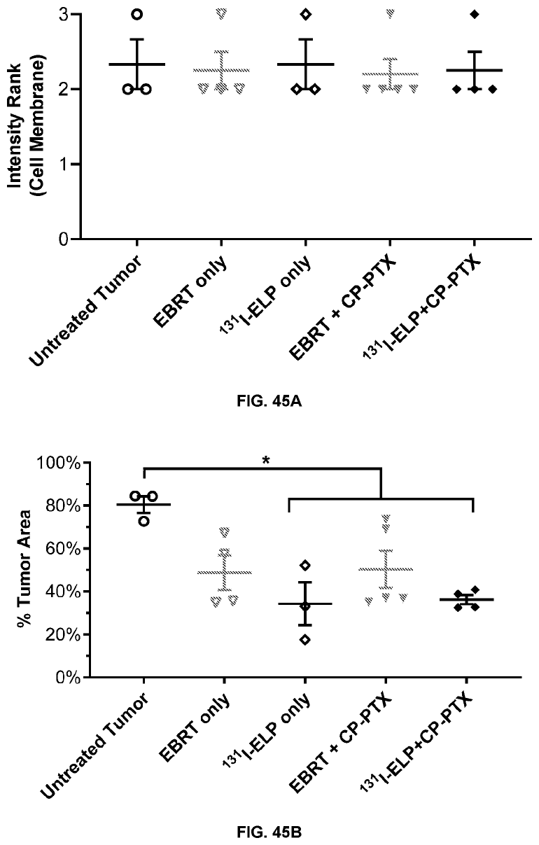

All of the treated tumors were then pathologically ranked and quantified (FIG. 45). It was immediately obvious that the intensity rank of viable cells was equivalent across all treatment groups: moderate-to-intense intensity. However, when the positive Claudin-4 stain was converted into a binary mask and quantified as a relative total area of each tumor specimen, some differential responses did emerge. ¹³¹I-ELP monotherapy, CP-PTX monotherapy, and ¹³¹I-ELP combination therapy all showed a significant reduction in the relative tumor coverage area when compared to untreated tumors. This was not unsurprising as the areas of high apoptosis appeared to have less intense staining compared to the remainder of the tumor. While not definitive, Claudin-4 expression did appear to be affected by the various treatments being applied to the tumors. As its main function is as a paracellular inhibitor of molecular diffusion, this suggested that drug permeability might be improved with these effects.

CD31 (PECAM-1) Analysis

The next vascular marker examined with immunohistology was CD-31. CD-31 is an antibody marker for platelet endothelial cell adhesion molecule 1 (PECAM-1). PECAM-1 cell surface expression is commonly upregulated across a wide range of cancers, including pancreatic tumors. It is specific to vascular endothelial cells, acting as a pro-angiogenic and pro-tumorigenic factor by suppressing mitochondrial-dependent apoptosis via the AKT/PKB pathway. Due to this, it has been implicated in conferring resistance to chemotherapeutics.

Moreover, it has been repeatedly shown to be upregulated in tumors after receiving radiation treatment. For these reasons, it was identified as an interesting microenvironment molecule to pathologically examine after comparative treatment.

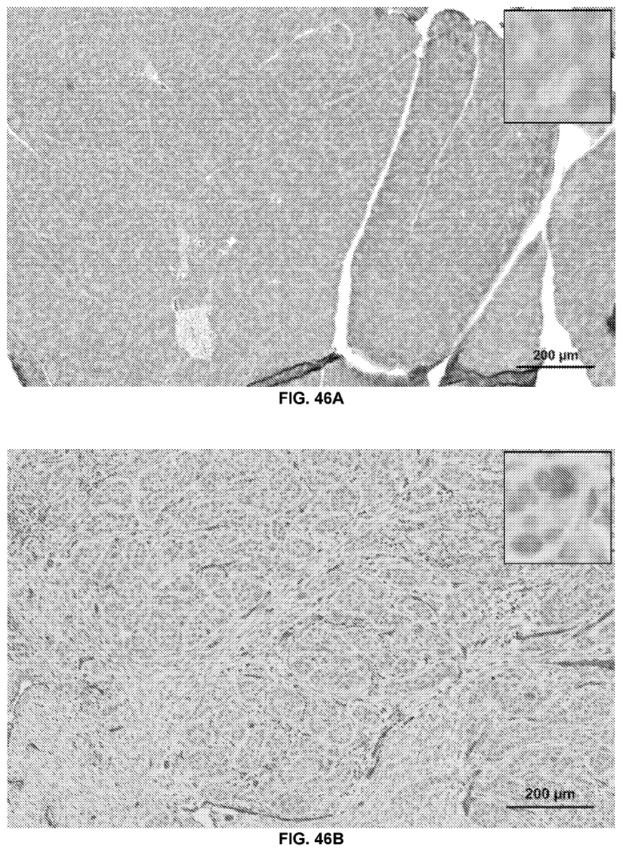

Normal pancreatic tissue excised from healthy mice showed a fairly standard pattern with positive luminal CD31 staining around vessels but negative ductal expression (FIG. 46A). This pattern shifted considerably in the BxPc3-luc2 tumors. Instead, untreated tumors displayed stromal staining with light cytoplasmic expression (FIG. 46B). The relative intensity of this staining was light.

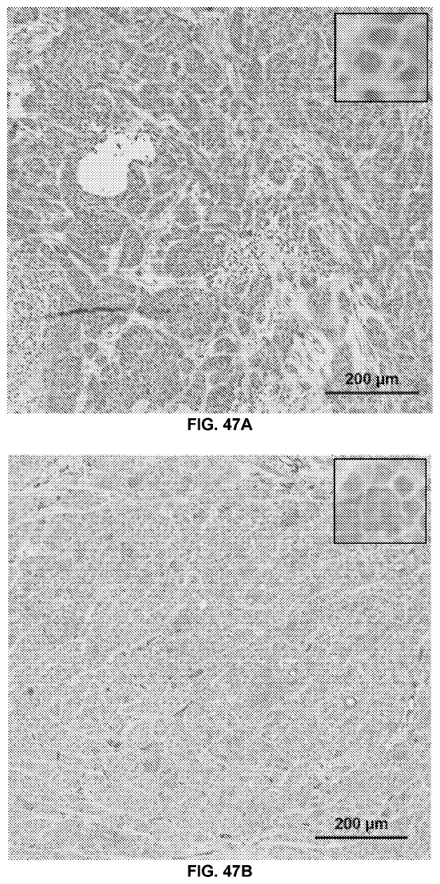

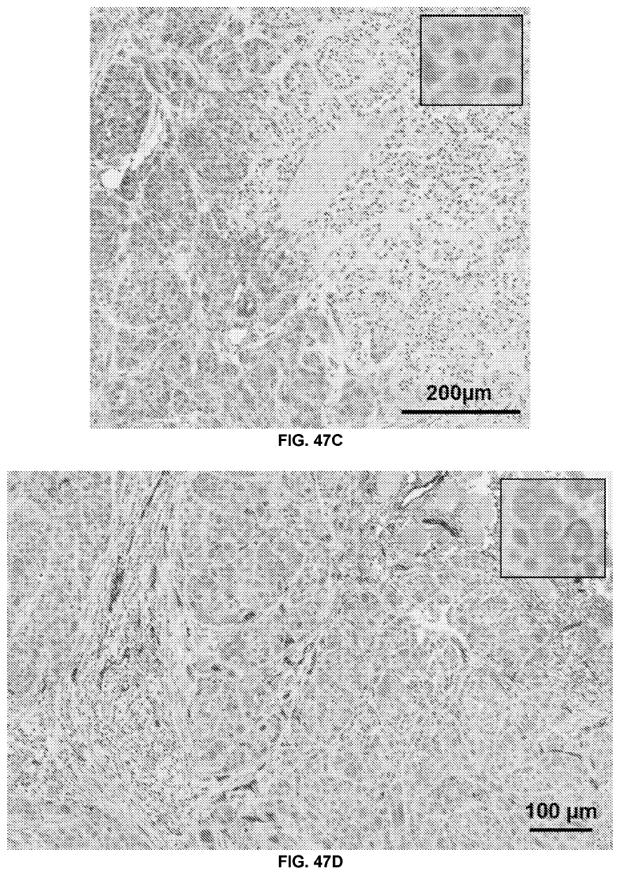

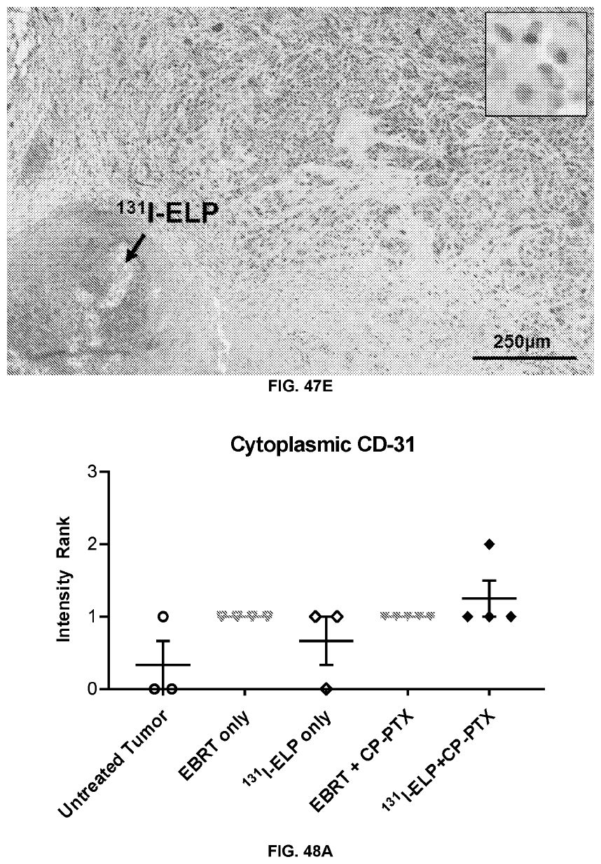

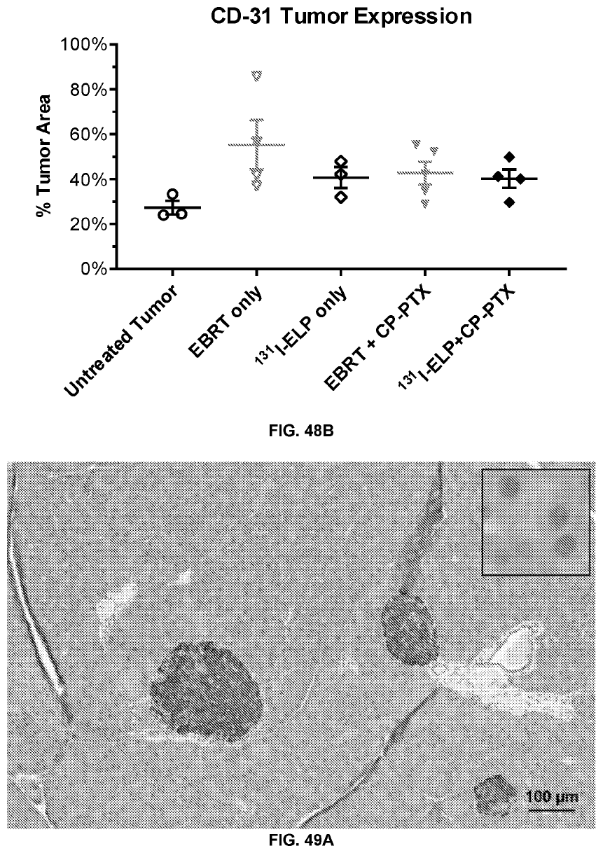

When the various treatment specimens were examined, no change in the staining intensity was observed (FIG. 47). All tissues continued to exhibit stromal expression with light cytoplasmic staining. No significant difference in the relative coverage area was observed. Only a minor difference was observed in the ¹³¹I-ELP combination therapy group. Areas of minimal, non-specific patterns of CD31 staining were seen around the depots. However, proper interpretation of these features could not be assessed because of the high levels of previously identified apoptosis (FIG. 48).

CD144 (VE-Cadherin) Analysis

The last junction protein that was used for histological analysis of the vascular permeability microenvironment was VE-Cadherin. VE-Cadherin is a glycoprotein that acts as a classical adherens junction protein. It consists of a single transmembrane region and maintains the integrity of the vascular endothelial barrier in a calcium-dependent manner. It is commonly upregulated in breast, pancreatic, and melanoma tumors. It has also been shown to activate phosphatidylinotisol 3-kinase, which can inhibit cellular apoptosis. Aberrant expression has been implicated in promoting malignancy via the endothelial-to-mesenchymal transition pathway. In histology, it can be stained for using the CD-144 antibody.

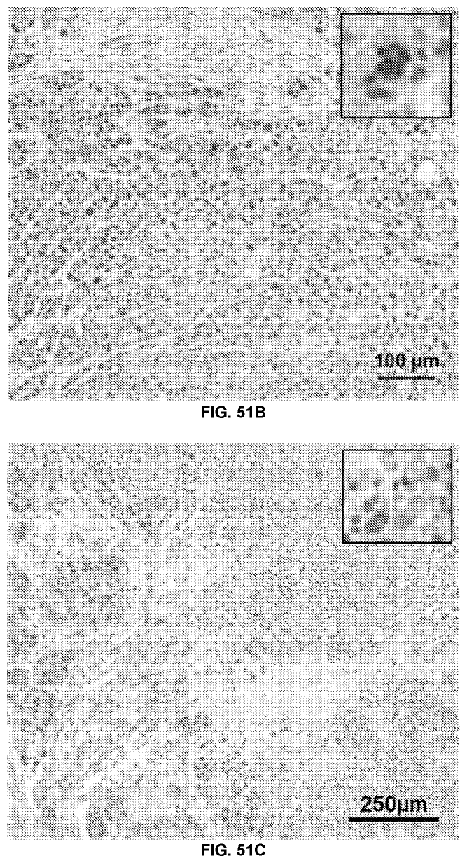

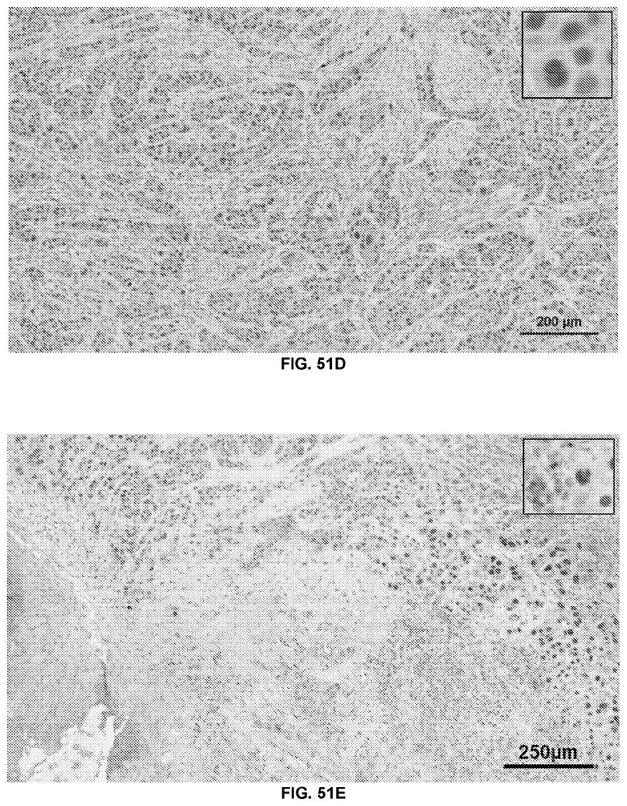

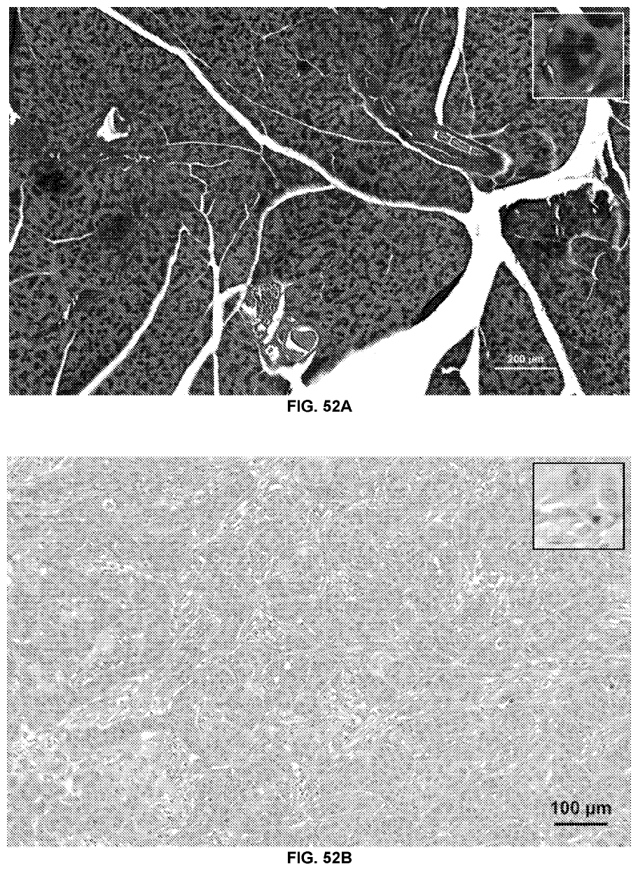

When the tumor specimens were stained with CD144, the normal murine pancreatic tissue was almost entirely negative for VE-Cadherin (FIG. 49A). The exception was found in the Langerhans islets, which showed strong positive staining. Untreated BxPc3 tumors were dramatically different (FIG. 49B). The cancerous cells showed CD-144 positivity in the nuclei, coupled with weak cytoplasmic expression. No relevant stromal expression was observed. This provided a baseline for comparing the remaining treatment specimens (FIG. 51).

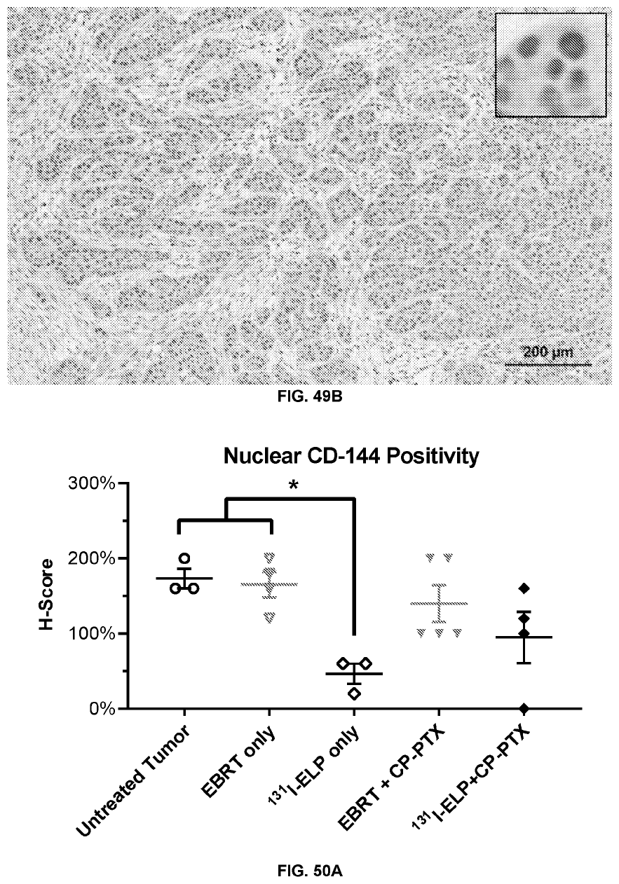

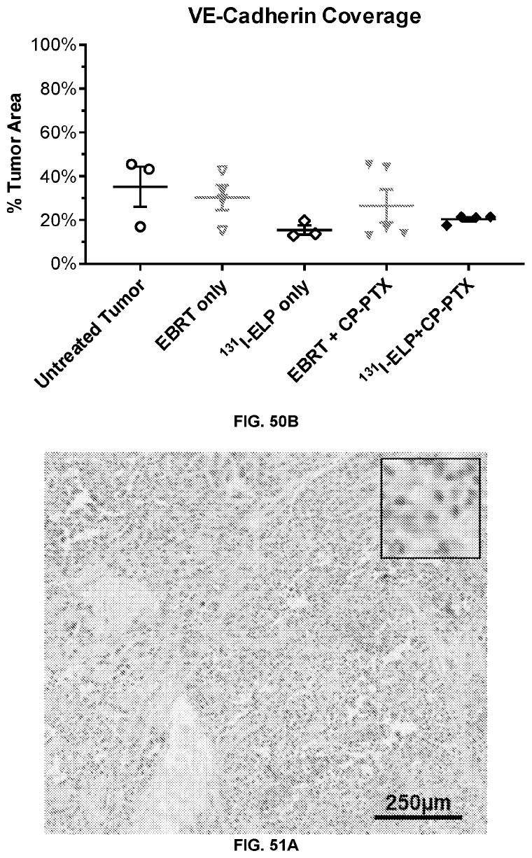

First the monotherapy tumor tissues were examined. Treatment with CP-PTX showed no distinguishable difference in the staining pattern from the untreated tumor—strong positivity in the nuclei and weak in the cytoplasm. X-ray EBRT also exhibited the same staining pattern with no difference in intensity. ¹³¹I-ELP only tumors showed slightly different histological features. The majority of these tumor tissues resembled the pattern of the untreated group. However, the pattern became disrupted in close proximity to the identifiable depot spots. In these regions, the nuclear foci became negative. To assess these differences, a histology score (H-score) was created by multiplying the relative intensity of the nuclear staining by the cellular frequency (FIG. 50A). The result quantified significantly lower nuclear CD144 expression than found in untreated tumors and other monotherapy groups (p<0.05).

As with the other IHC stains, the combination therapy groups provided more interesting results. EBRT combination therapy did not show any difference in the overall staining pattern. The H-score of its nuclear staining also corresponded with the EBRT monotherapy group and the untreated tumor specimen. The ¹³¹I-ELP combination therapy seemed to exhibit the same heterogeneous pattern seen in its monotherapy group, but over a larger area surrounding the depot sites. When the area of CD-144 coverage was quantified, a definite trend in VE-Cadherin reduction could be observed. However, this effect was not found to be statistically significant (FIG. 50B).

Masson Trichrome (Stromal Collagen) Analysis

In addition to examining junction barrier proteins, the tumor specimens were also investigated with Masson Trichrome to evaluate effects to the interstitial stromal content. Pancreatic tumors are uniquely characterized by a dense desmoplastic stromal content that inhibits the uptake of chemotherapeutics and promotes resistance. Masson Trichrome is a tri-colored stain that marks cellular nuclei in purple, the cell cytoplasm in pink, and highlights collagen in blue. When this stain was applied to the healthy pancreas of a mouse, the tissue was characterized primarily by the nuclear and cytoplasm staining of the acini cells (FIG. 52A). Collagen was only evident in normal abundance surrounding blood vessels. The BxPc2-luc2 tumor, however, clearly displayed the prototypical phenotype of abundant interstitial stroma (FIG. 52B). Light-to-intermediate collagen staining permeated throughout all of the tumor tissue, accounting for over 25% of the total tissue content.

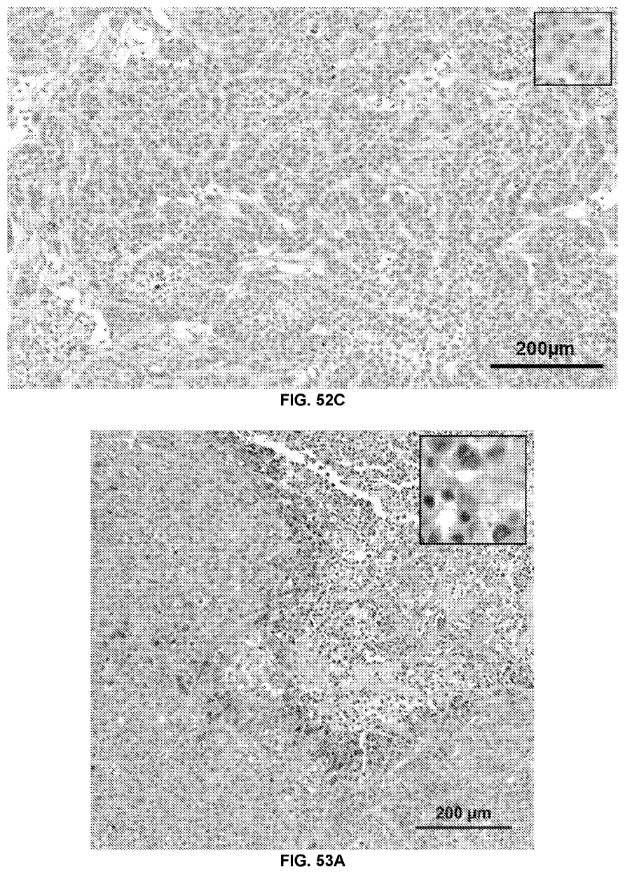

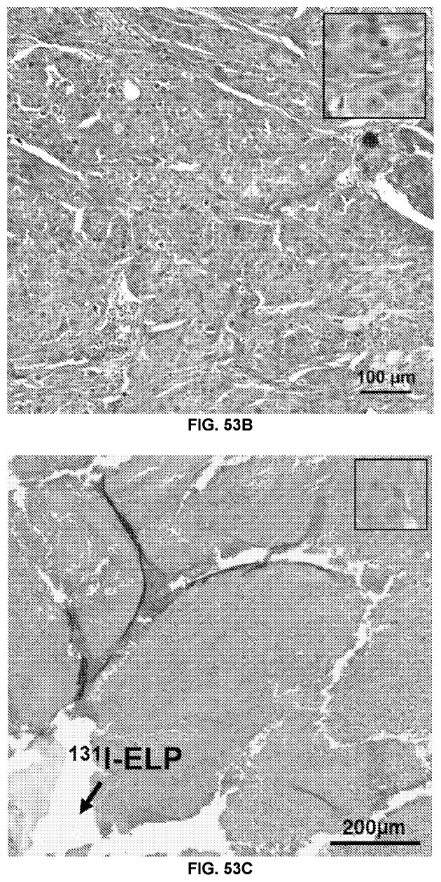

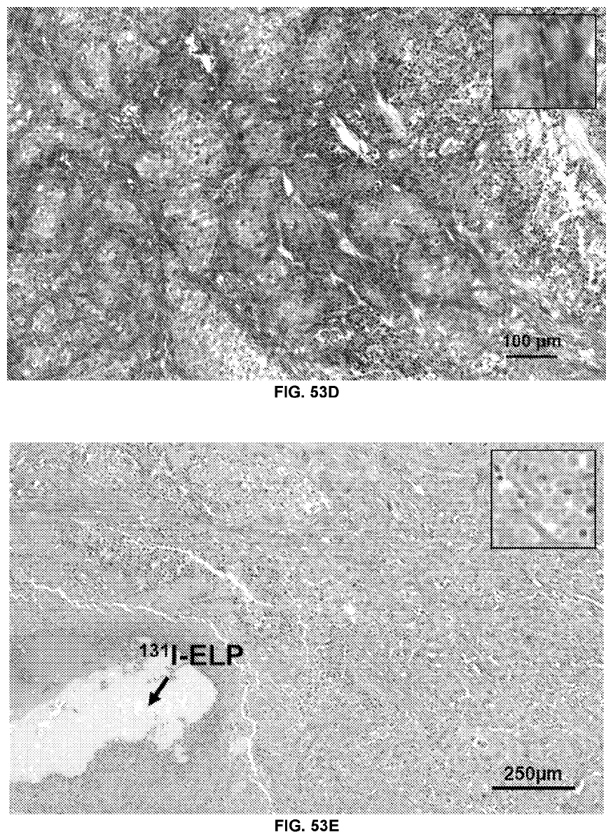

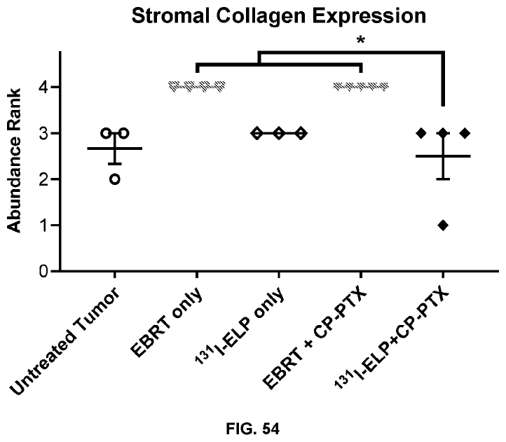

Unlike the vascular protein markers, each treatment group seemed to effect the composition of the BxPc3 stroma differently (FIG. 53). Only patches of the tumor cells seemed to be affected by the CP-PTX treatment. The typical stromal content remained unaffected. The specimens treated with only X-ray radiation actually demonstrated dense collagen, increased from that seen in the untreated tumor. This was not completely unexpected, as radiation has been clinically demonstrated to increase fibrosis in exposed tissues. The ¹³¹I-ELP-only treated specimens, however, did not exhibit increased collagen content. Instead, its collagen levels remained equivalent to the untreated tumor. There was a clear difference in the tumor tissue when EBRT was combined with CP-PTX. The tumor cells were highly inflamed with cytoplasmic leakage. However, the stromal collagen seemed unaffected by the combination treatment. Instead, it remained dense and intensely stained. Finally, the ¹³¹I-ELP combined with CP-PTX group demonstrated minimal-to-moderate collagen content. This was found to be significantly different than the intensity ranking of both external beam radiation groups (FIG. 54, p>0.05). The quality of the collagen varied according to the proximity to the ¹³¹I-ELP depot, as seen previously with the other histology stains. Collagen appeared dysregulated and fractured near the depot sites, while distant edges of the tumor retained the typical collagen patterning and stain intensity.

### Example 13: Tissue Histology of Orthotopic Specimens after Durable Tumor Regression

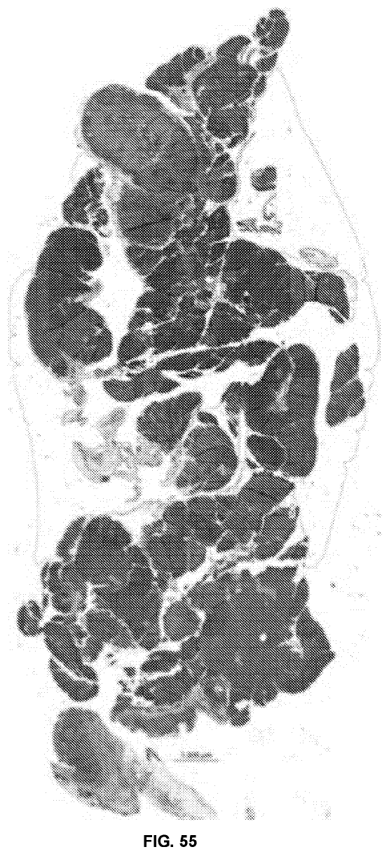

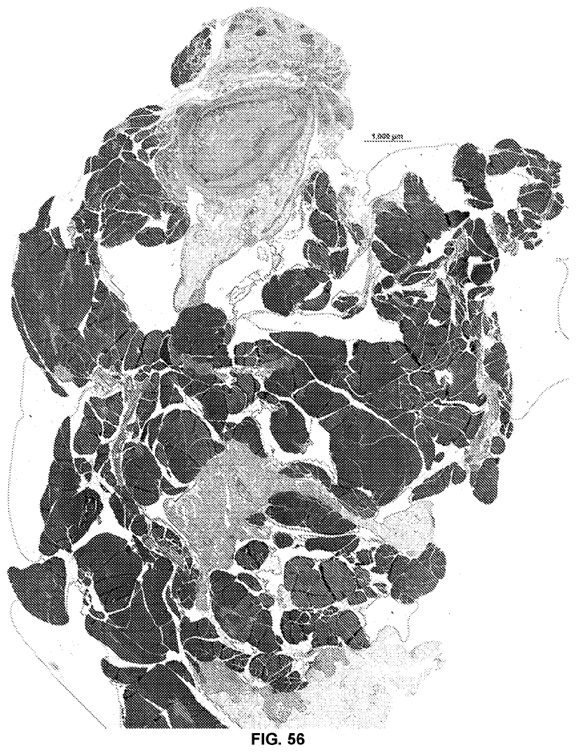

Two mice with orthotopic BxPc3-luc2 tumor were treated with ¹³¹I-ELP combination therapy and achieved full remission as observed with bioluminescent imaging. After 105 days, tumors remained in remission, and the pancreatic tissue was extracted for histological examination. The images (FIG. 55 and FIG. 56) from each are shown with the corresponding pathology analysis.

What is claimed is:

1. A composition comprising:
a first collection of self-assembling conjugates comprising at least one radionuclide coupled to a first elastin-like polypeptide; and
a chemotherapeutic.

2. The composition of claim 1, wherein the first elastin-like polypeptide comprises an amino acid sequence of (VPGX¹G)ₚ (SEQ ID NO: 3), wherein X¹ is any amino acid, p is 1 to 500 and wherein the elastin-like polypeptide has a transition temperature below about 42° C.

3. The composition of claim 1 or claim 2, wherein the first elastin-like polypeptide comprises an amino acid sequence of (VPGX²G)_{q}(GᵣY)ₛ (SEQ ID NO: 4), wherein X² is any amino acid, q is 1 to 500, r is 0-10, and s is 1-250 and wherein the elastin-like polypeptide has a transition temperature below about 42° C.

4. The composition of any one of claims 1-3, wherein the first elastin-like polypeptide comprises an amino acid sequence of (VPGVG)ₘ(GY)ₙ (SEQ ID NO: 1), wherein m is 50-250 and n is 1 to 50.

5. The composition of claim 4, wherein m is 120 and n is 7.

6. The composition of any one of claims 1-5, wherein the radionuclide provides irradiation through beta-particles, alpha-particles, gamma rays or Auger electrons.

7. The composition of any one of claims 1-6, wherein the radionuclide is ¹³¹Cesium, ¹³⁷Cesium, ⁶⁰Cobalt, ¹⁹²Iridium, ¹²⁵Iodine, ¹³¹Iodine, ¹⁰³Palladium, ¹⁰⁶Ruthenium, ²²³Radium, ²²⁶Radium, ⁹⁰Yttrium, ¹⁷⁷Lutetium, ¹¹¹Indium, ¹⁸⁶Rhenium, ⁸⁹Strontium, ¹⁵³Samarium, ³²Phosphorous, ²²⁵Actinium, ²¹¹Astatine, ²¹³Bismuth, or ²¹²Lead.

8. The composition of any one of claims 1-7, wherein the radionuclide is ¹³¹Iodine.

9. The composition of any one of claims 1-8, wherein the first collection of self-assembling conjugates are micelles.

10. The composition of any one of claims 1-9, wherein the elastin-like polypeptide has a critical micelle temperature below about 23° C. and a micelle-coacervation transition temperature below about 42° C.

11. The composition of any one of claims 1-10, wherein the chemotherapeutic is chosen from alkylating agents, anthracyclines, cytoskeletal disruptors or taxanes, epothilones, histone deacetylase inhibitors, topoisomerase inhibitors, kinase inhibitors, nucleotide analogs and precursor analogs, peptide antibiotics, platinum-based agents, retinoids, vinca alkaloids and derivatives, or combinations thereof.

12. The composition of any one of claims 1-11, wherein the chemotherapeutic is a cytoskeletal disruptor or taxane.

13. The composition of any one of claims 1-12, wherein the chemotherapeutic is paclitaxel.

14. The composition of any one of claim 1-13, wherein the chemotherapeutic is contained in a second collection of self-assembling conjugates, wherein the second collection of self-assembling conjugates comprises the chemotherapeutic coupled to a second elastin-like polypeptide.

15. The composition of claim 14, wherein the second elastin-like polypeptide comprises an amino acid sequence of SKGPG(X³GVPG)ₓWPC(GGC)_{z} (SEQ ID NO:2), wherein X³ is an amino acid or a combination of amino acids, x is 40 to 400 and z is 1 to 50.

16. The composition claim 15, wherein X³ is V:G:A in a ratio of 1:7:8, x is 160 and z is 7.

17. The composition of any one of claims 14-16, wherein the second collection of self-assembling conjugates are micelles.

18. A method of killing multiple cancer cells comprising contacting multiple cancer cells with the composition of any of claims 1-17.

19. A method of treating cancer comprising administering to a subject in need thereof a therapeutically effective amount of the composition of any of claims 1-17.

20. The method of claim 19, wherein the cancer is a solid tumor.

21. The method of claim 19 or claim 20, wherein the cancer is a pancreatic cancer, a prostate cancer, a breast cancer, a colorectal cancer, a cervical cancer, an ovarian cancer, a sarcoma, or a melanoma.

22. A method of treating cancer in a subject in need thereof, the method comprising co-administration of:
a therapeutically effective amount of a composition comprising a first collection of self-assembling conjugates comprising at least one radionuclide coupled to a first elastin-like polypeptide; and
a therapeutically effective amount of chemotherapeutic.

23. The method of claim 22, wherein the cancer is a solid tumor.

24. The method of claim 22 or claim 23, wherein the cancer is a pancreatic cancer, a prostate cancer, a breast cancer, a colorectal cancer, a cervical cancer, an ovarian cancer, a sarcoma, or a melanoma.

25. The method of any of claims 22-24, wherein the co-administration is simultaneous, separate or sequential.

26. The method of any of claims 22-25, wherein the composition and the chemotherapeutic are administered locally.

27. The method of any of claims 22-25, wherein the chemotherapeutic is administered systemically.

28. The method of any of claims 22-27, wherein the chemotherapeutic is administered in multiple doses.

29. The method of any of claims 22-28, wherein the first elastin-like polypeptide comprises an amino acid sequence of (VPGX¹G)ₚ (SEQ ID NO: 3), wherein X is any amino acid, p is 1 to 500 and has a transition temperature below about 42° C.

30. The method of any of claims 22-29, wherein the first elastin-like polypeptide comprises an amino acid sequence of (VPGX²G)_{q}(GᵣY)ₛ (SEQ ID NO: 4), wherein X² is any amino acid, q is 1 to 500, r is 0 to 10, s is 1-250 and has a transition temperature below about 42° C.

31. The method of any of claims 22-30, wherein the first elastin-like polypeptide comprises an amino acid sequence of (VPGVG)ₘ(GY)ₙ (SEQ ID NO: 1), wherein m is 50-250 and n is 1 to 50.

32. The method of claim 31, wherein m is 120 and n is 7.

33. The method of any of claims 22-32, wherein the radionuclide provides irradiation through beta-particles, alpha-particles, Gamma rays or Auger electrons.

34. The method of any one of claims 22-33, wherein the radionuclide is ¹³¹Cesium, ¹³⁷Cesium, ⁶⁰Cobalt, ¹⁹²Iridium, ¹²⁵Iodine, ¹³¹Iodine, ¹⁰³Palladium, ¹⁰⁶Ruthenium, ²²³Radium, ²²⁶Radium, ⁹⁰Yttrium, ¹⁷⁷Lutetium, ¹¹¹Indium, ¹⁸⁶Rhenium, ⁸⁹Strontium, ¹⁵³Samarium, ³²Phosphorous, ²²⁵Actinium, ²¹¹Astatine, ²¹³Bismuth, or ²¹²Lead.

35. The method of any one of claims 22-34, wherein the radionuclide is ¹³¹Iodine.

36. The method of any one of claims 22-35, wherein the first collection of self-assembling conjugates are micelles.

37. The method of any one of claims 22-36, wherein the elastin-like polypeptide has a critical micelle temperature below about 23° C. and a micelle-coacervation transition temperature below about 42° C.

38. The method of any one of claims 22-37, wherein the chemotherapeutic is chosen from alkylating agents, anthracyclines, cytoskeletal disruptors or taxanes, epothilones, histone deacetylase inhibitors, topoisomerase inhibitors, kinase inhibitors, nucleotide analogs and precursor analogs, peptide antibiotics, platinum-based agents, retinoids, vinca alkaloids and derivatives, or combinations thereof.

39. The method of any one of claims 22-38, wherein the chemotherapeutic is a cytoskeletal disruptor or taxane.

40. The method of any one of claims 22-39, wherein the chemotherapeutic is paclitaxel.

41. The method of any one of claim 22-40, wherein the chemotherapeutic is contained in a second collection of self-assembling conjugates, wherein the second collection of self-assembling conjugates comprises the chemotherapeutic coupled to a second elastin-like polypeptide.

42. The method of claim 41, wherein the second elastin-like polypeptide comprises an amino acid sequence of SKGPG(X³GVPG)ₓWPC(GGC)_{z} (SEQ ID NO:2), wherein X³ is an amino acid or a combination of amino acids, x is 40 to 400 and z is 1 to 50.

43. The method of claim 42, wherein X³ is V:G:A in a ratio of 1:7:8, x is 160 and z is 7.

44. The method of any one of claims 41-43, wherein the second collection of self-assembling conjugates are micelles.


SEQUENCE LISTING (4 sequences)

SEQ ID NO: 1 - protein, 7 aa, Artificial Sequence
  Synthetic
  REPEAT (1)..(5): Repeats 50 to 250 times
  REPEAT (6)..(7): Repeats 1 to 50 times
VPGVGGY

SEQ ID NO: 2 - protein, 16 aa, Artificial Sequence
  Synthetic
  Xaa (6)..(6): Amino acid or a combination of amino acids
  REPEAT (6)..(10): Repeats 40 to 400 times
  REPEAT (14)..(16): Repeats 1 to 50 times
SKGPGXGVPGWPCGGC

SEQ ID NO: 3 - protein, 5 aa, Artificial Sequence
  Synthetic
  REPEAT (1)..(5): Repeats 1 to 500 times
  MISC_FEATURE (1)..(5): Has a transition temperature below about 42 degrees celsius
  Xaa (4)..(4): Any amino acid
VPGXG

SEQ ID NO: 4 - protein, 7 aa, Artificial Sequence
  Synthetic
  REPEAT (1)..(5): Repeats 1 to 500 times
  Xaa (4)..(4): Any amino acid
  REPEAT (6)..(6): Repeats 0 to 10 times
  REPEAT (6)..(7): Repeats 1 to 250 times
  MISC_FEATURE (6)..(7): Has a transition temperature below about 42 degrees celcius
VPGXGGY